(19) United States
(12) Patent Application Publication
(10) Pub. No.: US 2024/0254087 A1
(43) Pub. Date: Aug. 1, 2024

(54) SELECTIVE, PARTIAL, AND ARRESTIN-BIASED 5-HT2A AGONISTS WITH UTILITY IN VARIOUS DISORDERS

(71) Applicants: Saint Joseph's University, Philadelphia, PA (US); The Medical College of Wisconsin, Inc., Milwaukee, WI (US); The Regents of the University of California, Oakland, CA (US)

(72) Inventors: Jason WALLACH, Philadelphia, PA (US); John MCCORVY, Milwaukee, WI (US); Adam HALBERSTADT, Oakland, CA (US)

(21) Appl. No.: 18/560,113

(22) PCT Filed: May 11, 2022
(86) PCT No.: PCT/US2022/028797

§ 371 Date: Nov. 10, 2023

Related U.S. Application Data
(60) Provisional application No. 63/186,988, filed on May 11, 2021.

(51) Int. Cl. C07D 215/12; A61K 31/135; A61K 31/341; A61K 31/343; A61K 31/357; A61K 31/381; A61K 31/415; A61K 31/44; A61K 31/472; A61K 45/06; C07C 217/60; C07D 213/38; C07D 217/04; C07D 231/12; C07D 307/52; C07D 307/91; C07D 317/64; C07D 333/20; C07D 333/76; C07D 493/04

(52) U.S. Cl. CPC C07D 215/12; A61K 31/135; A61K 31/341; A61K 31/343; A61K 31/357; A61K 31/381; A61K 31/415; A61K 31/44; A61K 31/472; A61K 45/06; C07C 217/60; C07D 213/38; C07D 217/04; C07D 231/12; C07D 307/52; C07D 307/91; C07D 317/64; C07D 333/20; C07D 333/76; C07D 493/04; C07C 2602/08; C07C 2602/10

(57) ABSTRACT

Disclosed herein are novel serotonin 5-HT_{2A} receptor agonists with selectivity for the 5-HT_{2A} receptor subtype over other serotonin receptors. Some of these 5-HT_{2A} agonists exhibit functional selectivity and preferentially activate arrestin signaling over G protein-mediated signaling. Also disclosed are pharmaceutical compositions of the compounds and methods of treating certain diseases or conditions.

### CROSS-REFERENCE TO RELATED APPLICATIONS

This application claims the benefit of U.S. Provisional Patent Application No. 63/186,988, filed on May 11, 2021, the contents of which are herein incorporated by reference into the subject application.

### STATEMENT OF GOVERNMENT INTEREST

This invention was made with government support under grant numbers R35 GM133421 and R01 DA041336 awarded by the National Institute of Health. The government has certain rights in this invention.

### TECHNICAL FIELD

Disclosed herein are novel serotonin 5-HT_{2A} receptor agonists with selectivity for the 5-HT_{2A} receptor over other serotonergic and non-serotonergic receptors.

### BACKGROUND

Psychedelic drugs (also known as classical hallucinogens) can be divided into the lysergamide, tryptamine, and phenylalkylamine structural classes. These agents act as agonists of the 5-HT_{2A} receptor, but also have off-target effects at other 5-HT receptors including 5-HT_{2B}. Numerous recent investigations have highlighted the impressive clinical efficacy of 5-HT_{2A} receptor targeting drugs for many therapeutic indications, including depression, inflammatory disease, addiction as well as numerous other systemic diseases, psychiatric disorders, and neurological conditions. In some cases, however, the hallucinogenic activities of these agents may limit their therapeutic potential, especially for certain indications. Another class of 5-HT_{2A} related drugs are inverse agonists and neutral antagonists, which have been developed as antipsychotics, antidepressants, and hypnotics, amoung other therapeutic indications. Many of these ligands are not selective or have undersirable properties resulting from their lack of 5-HT_{2A} selectivity and/or functional selectivity for distinct 5-HT_{2A} signaling pathways.

Thus, there is a need to develop new agents that exhibit therapeutic effects similar to existing ligands but are less prone to induce undesirable side-effects, including hallucinogenic activity, altered cognition and affect, and cardiotoxicty.

### SUMMARY

This patent disclosure provides G protein partial agonists and functionally selective and/or biased arrestin agonists for the 5-HT_{2A} receptor. Biased 5-HT_{2A} agonists are ligands capable of selectively activating a subset of the signaling pathways downstream from the 5-HT_{2A} receptor, which allows the compounds to produce desirable therapeutic effects with a reduction or absence of undesirable side-effects. Partial agonists and functionally selective or biased agonists can be applied to the treatment of various diseases and conditions. They can also be used to modify or attenuate the effects produced by a hallucinogenic drug in an animal or human.

An aspect of the patent document provides a compound of formula (I) or a pharmaceutically acceptable salt thereof,

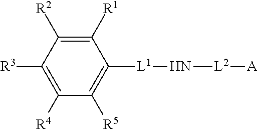

Wherein:

R¹, R², R³, R⁴ and R⁵ are independently selected from the group consisting of hydrogen, deuterium, OC₁₋₆alkyl, SC₁₋₆alkyl, CN, OH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxy C₁₋₁₀alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, C₃₋₆cycloalkyl, 3-10 membered heterocycloalkyl, 6-10 membered aryl, 5-10 membered heteroaryl, C₁₋₆alkylene-N(R^{m})₂, C₁₋₆alkylene-N(R^{m})(COR^{m}); wherein two adjacent substituents may link up to form a ring:
provided that at least one of R¹ and R² contains an oxygen bonded to the phenyl ring:
A is a 4, 5, 6 or 7 membered ring optionally substituted with one or more substituents selected from the group consisting of OC₁₋₆alkyl, SC₁₋₆alkyl, CN, OH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxy C₁₋₆alkyl, dihydroxy C₁₋₁₀alkyl, C₃₋₆cycloalkyl, 3-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl_{6-10-membered}, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroaryl_{5-10-membered}, OC₁₋₂alkylene-bicycloalkyl_{5-12-membered}, OC₁₋₂alkylene-hetero-bicycloalkyl_{5-12-membered}, C₁₋₂alkylene-C₃₋₆cycloalkyl, C₁₋₂alkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, C₁₋₂alkylene-hetero-bicycloalkyl_{5-12-membered}, wherein the C₃₋₆cycloalkyl, 3-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl_{6-10-membered}, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroaryl_{5-10-membered}, OC₁₋₂alkylene-bicycloalkyl_{5-12-membered}, OC₁₋₂alkylene-hetero-bicycloalkyl_{5-12-membered}, C₁₋₂alkylene-C₃₋₆cycloalkyl, Cizalkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, and C₁₋₂alkylene-hetero-bicycloalkyl_{5-12-membered} are optionally substituted with one or more substituents selected from the group consisting of OC₁₋₆alkyl, SC₁₋₆alkyl, CN, N₃, OH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, haloC₁₋₆alkylene-O, C₁₋₆alkyl, hydroxy C₁₋₆alkyl, C(═NC₁₋₆alkyl)C₁₋₆alkyl, OC(O)N(R^{m})₂, SH, C(O)SR^{m}, OC₁-balkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC1-6alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂;
alternatively, two adjacent substituents of A link up and together with A form a bicyclic or tricyclic ring:
R^{m} each is independently hydrogen or C₁₋₆alkyl or halo-C₁₋₆alkyl:
L¹ is C₁₋₃alkylene; and
L² is a bond or C₁₋₃alkylene optionally substituted with C₁₋₄alkyl, C₃₋₆ cycloalkyl, haloC₁₋₄alkyl, deuterium or F.

In some embodiments, the compound is not

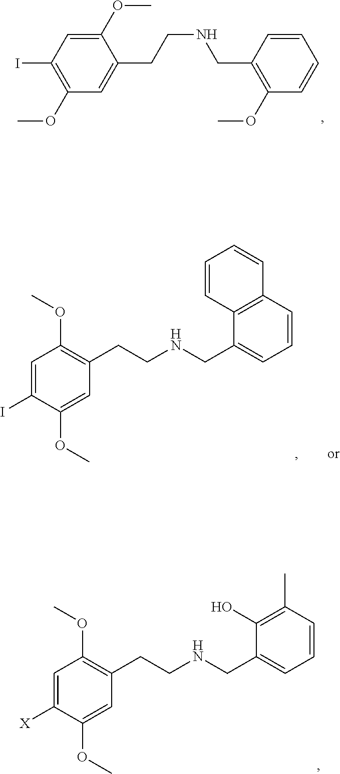

wherein X is CN, Cl, Br or I.

Also provided are pharmaceutical compositions comprising a therapeutically effective amount of a compound of Formula (I) disclosed herein or pharmaceutically acceptable salt thereof, and a pharmaceutically acceptable excipient.

Another aspect provides a method for treating a disease or condition. The method includes administering to a subject in need thereof a compound of formula (I), a pharmaceutically acceptable salt thereof, or a pharmaceutical composition thereof. In some embodiments, the disease or condition is a psychiatric or neurological disease or condition.

Another aspect provides a method to selectively activate β-arrestin-dependent pathways over G protein-dependent pathways. The method involves contacting a serotonin 5-HT_{2A} receptor with the compound, a pharmaceutically acceptable salt thereof, or a pharmaceutical composition thereof.

### DESCRIPTION OF THE DRAWINGS

FIG. 1 shows that 5-HT_{2A} arrestin biased agonist ligands block the psychedelic-like activity (as measured by head-twitch response, HTR) of other 5-HT_{2A} agonists and block hyperlocomotion produced by the dissociative N-methyl-D-aspartate receptor antagonist, phencyclidine (PCP). (A). DOI, a hallucinogen and 5-HT_{2A} agonist, was administered IP 10 min after SC administration of vehicle or test drug. A and then HTR activity was assessed for 30 minutes. *p<0.001, significant difference vs. vehicle (B). Vehicle or the NMDA receptor antagonist PCP (5 mg/kg) was injected IP 10 min after SC administration of vehicle or 25N-NB1-Nap (16), and then the mice were placed in the test chambers 10 min later for a 60-min assessment of locomotor activity. (C). 5-HT_{2A} antagonist M100907 pretreatment blocks PCP induced hyperactivity in mice (M100907×PCP: F_{1,20}=7.41, p=0.0131). Mice were treated with vehicle or M100907 (0.03 mg/kg SC) 10 min prior to vehicle or PCP (5 mg/kg IP): animals were placed in the test chambers 10 min after treatment with PCP. Animals were tested for 60 min. *p<0.01 vs. vehicle: ^{#}p<0.01 vs. PCP alone. (D) Antagonism of 5-HT induced h5-HT_{2A} Ca²⁺ flux activation by 25N—NBPh (17) and 25N—N-1-Nap (16).

FIG. 2 shows mouse HTR for representative compounds showing no significant elevation of HTR over baseline with multiple doses.

FIG. 3 shows tolerance to DOI or 25N—N1-Nap (16) but not inverse agonist Pimavanserin.

FIG. 4 shows ¹³C NMR Chemical Shift Assignments of example compounds.

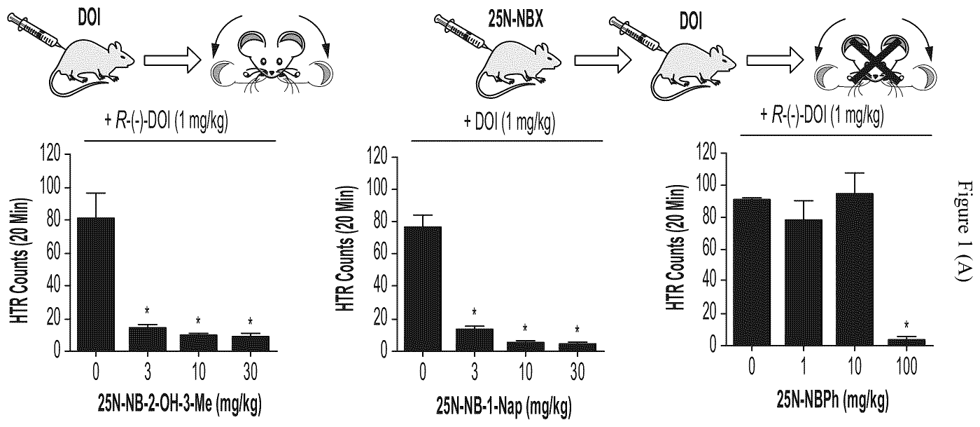

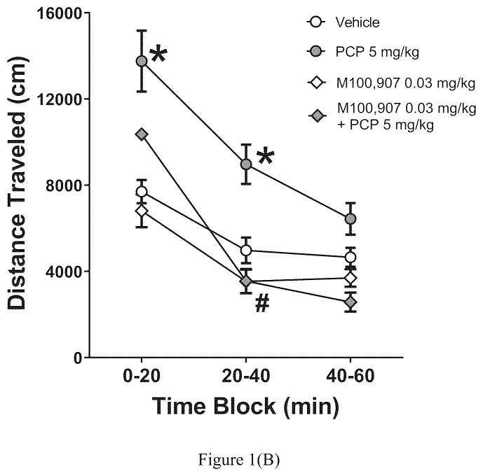

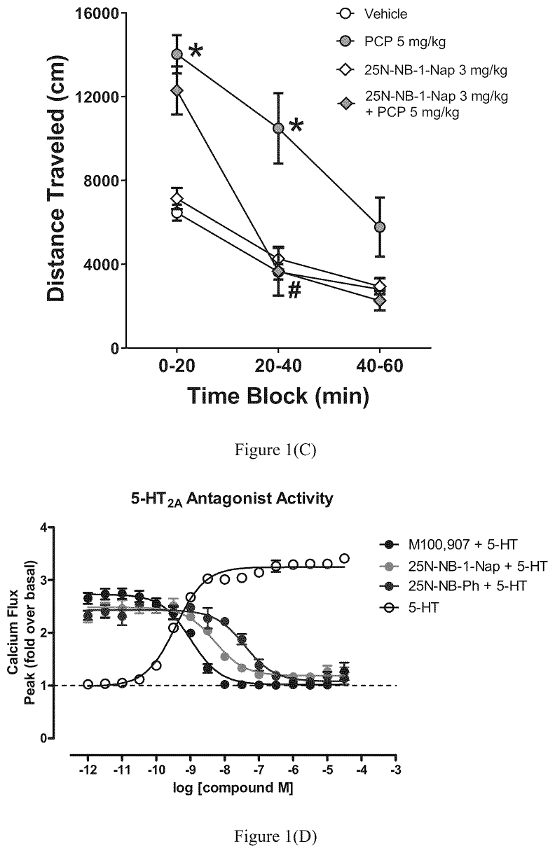

### DETAILED DESCRIPTION

This patent specification discloses serotonin 5-HT_{2A} receptor agonists with selectivity for the 5-HT_{2A} receptor subtype and/or the 5-HT_{2B}, and/or the 5-HT_{2C} receptor, and combinations thereof, over other serotonin receptors (notably over 5-HT_{2B}, a receptor known to mediate drug-induced cardiotoxicity). Some of the compounds are G protein partial agonists, whereas others show functional signaling bias for activating the β-arrestin pathway with little or no stimulation of G protein activity relative to other compounds in these series and known 5-HT_{2A} agonists like 25I—NBOMe, LSD, 5-MeO-DMT, DMT and 2C—I. These G protein partial agonists and functionally selective β-arrestin agonists show a lack of hallucinogenic-like behavioral responses as measured in mice. Furthermore, they block hallucinogen-like behavorial effects induced by known hallucinogens like DOI and induce antipsychotic-like effects in animal models. These compounds not only have the capacity to work as novel antipsychotic and antidepressant medications but also have therapeutic potential for many other diseases and conditions.

While the following text may reference or exemplify specific embodiments of a compound, substituent, or use thereof, it is not intended to limit the scope of the compound, substituent or its use to such particular references or examples. Various modifications may be made by those skilled in the art, in view of scientific and practical considerations, such as replacement of a substituent or treatment of other diseases.

The articles “a” and “an” as used herein refer to “one or more” or “at least one,” unless otherwise indicated. That is, reference to any element or component of an embodiment by the indefinite article “a” or “an” does not exclude the possibility that more than one element or component is present.

The term “about” as used herein refers to the referenced numeric indication plus or minus 10% of that referenced numeric indication. In some embodiments, the term “about” refers to the referenced numeric indication plus or minus 5% of that referenced numeric indication.

The term “acyl” refers to —C(O)CH₃, —C(O)CH₂CH₃, —C(O)CH₂CH₂CH₃, or —C(O)CH₂CH₂CH₂CH₃.

The term “alkyl” refers to a hydrocarbon or a hydrocarbon chain which may be either straight-chained or branched. The term “C₁₋₆ alkyl” refers to alkyl groups having 1, 2, 3, 4, 5 or 6 carbon atoms. Non-limiting examples include groups such as CH₃, (CH₂)₂CH₃, CH₂CH(CH₃)CH₃, and the like. Similarly, the term “C₂₋₅ alkyl” refers to alkyl groups having 2, 3, 4 or 5 carbon atoms.

The term “alkylene” refers to a divalent hydrocarbon or a hydrocarbon chain which may be either straight-chained or branched. Non-limiting examples include groups such as CH₂, (CH₂)₂CH₂, CH₂CH(CH₃)CH₂, and the like. A C₁₋₃alkylene includes alkylenes with 1, 2 or 3 carbons such as CH₂, (CH₂)₂, (CH₂)₃, and CH(CH₃)CH₂.

The term “cycloalkyl” refers to saturated and partially unsaturated cyclic hydrocarbon groups having 3 to 12 ring carbons, for example 3 to 8 carbons, and as a further example 3 to 6 carbons, wherein the cycloalkyl group additionally is optionally substituted. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “aryl” group refers to a C₆₋₁₄ aromatic moiety comprising one to three aromatic rings, which is optionally substituted. Examples of aryl groups include, without limitation, phenyl, naphthyl, anthracenyl, fluorenyl, and dihydrobenzofuranyl.

The term “alkeny” refers to a caron chain containing a carbon-carbon double bond moiety. Non-limiting examples of alkenyl groups include ethylenyl, 1-propenyl, allyl and 2-butenyl.

The term “alkynyl” group refers to a caron chain containing a carbon-carbon triple bond moiety. Non-limiting examples of alkynyl groups include ethynyl, 1-propanyl, propargyl and 2-butynyl.

The term “haloalkyl” refers to a C₁₋₁₀alkyl chain, straight or branched, in which one or more hydrogen has been replaced by a halogen. Non-limiting examples of haloalkyls include CHF₂, CFH₂, CF₃, CH₂CHF₂, CH₂CH₂Cl, CH₂CF₃, and CH₂CH₂F. In some embodiments, the alkyl in haloalkyl has 1, 2, 3 or 4 carbons.

The term “heteroalkyl” refers to a C₁₋₁₀alkyl group, straight or branched, wherein one or more carbon atoms in the chain are replaced by one or more heteroatoms selected from the group consisting of O, S, N and NR^{m}. In some embodiments, the alkyl in heteroalkyl has 1 to 10 carbons. In some embodiments, the alkyl in heteroalkyl has 2, 3, 4 or more than 2 carbons.

The term “hydroxyalkyl” refers to a C₁₋₁₀alkyl chain, straight or branched, wherein a carbon is substituted with a hydroxyl group. The carbon the hydroxyl is attached to is a primary carbon or secondary carbon. In some embodiments, the alkyl in hydroxylalkyl has 2, 3, 4 or more than 2 carbons.

The term “dihydroxyalkyl” refers to a C₂₋₁₀alkyl chain, straight or branched, wherein two carbons are each substituted with a hydroxyl group. In some embodiments, the alkyl in dihydroxylalkyl has 2, 3, 4 or more than 2 carbons.

The term “heterocyclyl” or “heterocyclic” group is a ring structure having from about 3 to about 12 atoms, for example 4 to 8 atoms, wherein one or more atoms are selected from the group consisting of N, O, and S, the remainder of the ring atoms being carbon. The heterocyclyl may be a monocyclic, a bicyclic, a spirocyclic or a bridged ring system. Examples of heterocyclic groups include, without limitation, epoxy, azetidinyl, aziridinyl, azocanyl, azepanyl, diazepanyl, dihydrofuranyl, tetrahydrofuranyl, tetrahydropyranyl, oxazepanyl, pyrrolidinyl, pyrrolidinonyl, piperidinyl, piperazinyl, imidazolidinyl, thiazolidinyl, thiooxazepanyl, dithianyl, trithianyl, dioxolanyl, oxazolidinyl, oxazolidinonyl, decahydroquinolinyl, piperidonyl, 4-piperidinonyl, thiomorpholinyl, thiomorpholinyl 1,1 dioxide, morpholinyl, oxazepanyl, azabicyclohexanes, azabicycloheptanes and oxa azabiocycloheptanes. Specifically excluded from the scope of this term are compounds having adjacent annular O and/or S atoms.

The term “heteroaryl” refers to groups having 5 to 14 ring atoms, preferably 5, 6, 9, or 10 ring atoms: having 6, 10, or 14 π electrons shared in a cyclic array; and having, in addition to carbon atoms, from one to three heteroatoms per ring selected from the group consisting of N, O, and S. Examples of heteroaryl groups include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

The term “halogen” refers to F, Cl, Br or I.

The term “subject” refers to humans or animals including for example sheep, horses, cattle, pigs, dogs, cats, rats, mice, birds, and reptiles. Preferably, the subject is a human or other mammal.

The term “effective amount” or “therapeutically effective amount” of a compound is an amount that is sufficient to ameliorate, or in some manner reduce a symptom or stop or reverse progression of a condition, or negatively modulate or inhibit activity. Such amount may be administered as a single dosage or may be administered according to a regimen, whereby it is effective.

The term “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

The term “pharmaceutically acceptable carrier” refers to a chemical compound that facilitates the delivery or incorporation of a compound or therapeutic agent into cells or tissues.

The term “pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid: or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxy benzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tannic acid, tartaric acid, tertiary butylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, ethylenediamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutical composition” refers to a mixture of a compound disclosed herein with other chemical components, such as diluents or additional carriers. The pharmaceutical composition facilitates administration of the compound to an organism. Multiple techniques of administering a pharmaceutical composition exist in the art including, but not limited to, oral, injection, aerosol, parenteral, intranasal, sublingual, inhalational, and topical administration. In some embodiments, pharmaceutically acceptable salts of the compounds disclosed herein are provided.

The term “treating” or “treatment” of any disease or condition refers, in some embodiments, to ameliorating the disease or disorder (i.e., arresting or reducing the development of the disease or at least one of the clinical signs and symptoms thereof). In some embodiments “treating” or “treatment” refers to ameliorating at least one physical parameter, which may not be discernible by the subject. In some embodiments, “treating” or “treatment” refers to modulating the disease or disorder, either physically, (e.g., stabilization of a discernible symptom), physiologically, (e.g., stabilization of a physical parameter), or both. In some embodiments, “treating” or “treatment” refers to delaying the onset of the disease or disorder, or even preventing the same. “Prophylactic treatment” is to be construed as any mode of treatment that is used to prevent progression of the disease or is used for precautionary purpose for persons at risk of developing the condition.

Compounds described in this patent specification exhibit a high degree of signaling preference for β-arrestin over G protein-dependent signaling pathways relative to other known 5-HT_{2A} agonists. Others activate G protein-dependent signaling pathways with reduced efficacy (Emax) relative to known hallucinogens like DMT, DOI, LSD and 2C—I. These compounds do not induce the HTR in mice (a behavior that is predictive of hallucinogenic effects in humans) and antagonize the HTR induced by the prototypical 5-HT_{2A} agonist and psychedelic drug 2,5-dimethoxy-4-iodoamphetamine (DOI). Some of these compounds also block phencyclidine (PCP)-induced hyperactivity in mice. 5-HT_{2A} agonists with weak-to-modest G protein efficacy or those that have a bias for activating β-arrestin show unique pharmacological profiles while lacking the hallucinogenic activity associated with other 5-HT_{2A} agonists.

An aspect of the disclosure provides a compound of formula I or a pharmaceutically acceptable salt thereof,

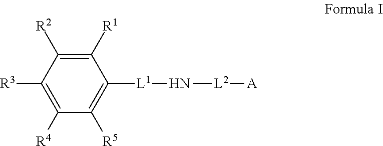

Wherein:
R¹, R², R³, R⁴ and R⁵ are independently selected from the group consisting of hydrogen, deuterium, OC₁₋₆alkyl, SC₁₋₆alkyl, CN, OH, halogen, N₃, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxy C₁₋₁₀alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alky leneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, C₃₋₆cycloalkyl, 3-10 membered heterocycloalkyl, 6-10 membered aryl, 5-10 membered heteroaryl, C₁₋₆alkylene-N(R^{m})₂, C₁₋₆alkylene-N(R^{m})(COR^{m}); wherein two adjacent substituents may link up to form a ring (e.g. furanyl, dihydrofuranyl, tetrahydrofuranyl, 1,3-dioxolanyl, etc.):
provided that at least one of R¹ and R² contains an oxygen bonded to the phenyl ring (e.g. OC₁₋₃alkyl, or R¹ and R² linked up to form a furanyl ring, etc);
A is 4 or 5 or 6 membered ring or a 7 to 10 membered bicyclic ring, wherein the ring optionally substituted with one or more substituents selected from the group consisting of OC₁₋₆alkyl, SC₁₋₆alkyl, CN, OH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxy C₁₋₆alkyl, dihydroxy C₁₋₁₀alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, C₃₋₆cycloalkyl, 3-10 membered heterocycloalkyl, 6-10 membered aryl, 5-10 membered heteroary 1, 5-12 membered bicycloalkyl, and 5-12 membered hetero-bicycloalkyl, wherein the C₃₋₆cycloalkyl, 3-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl_{6-10-membered}, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroaryl_{5-10-membered}, OC₁₋₂alkylene-bicycloalkyl_{5-12-membered}, OC₁₋₂alkylene-hetero-bicycloalkyl_{5-12-membered}, C₁₋₂alkylene-C₃₋₆cycloalkyl, C₁₋₂alkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, and C₁₋₂alkylene-hetero-bicycloalkyl_{5-12-membered} are optionally substituted with one or more substituents selected from the group consisting of OC₁₋₆alkyl, SC₁₋₆alkyl, CN, OH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂;
alternatively, two adjacent substituents of A link up to form an additional ring, which together with A form a bicyclic ring or a tricyclic ring;
R^{m} each is independently hydrogen or C₁₋₆alkyl or halo-C₁₋₆alkyl;
L¹ is C₁₋₃alkylen, optionally R¹ and L¹ link up to form a ring; and
L² is a bond or C₁₋₃alkylene optionally substituted with C₁₋₄alkyl, C₃₋₆ cycloalkyl, haloC₁₋₄alkyl, deuterium or halogen (e.g. F, Cl, Br, and I). In some embodiments, L² is a methylene optionally substituted with methyl, ethyl, propyl, isopropyl, F or deuterium.

In some embodiments, the compound is not

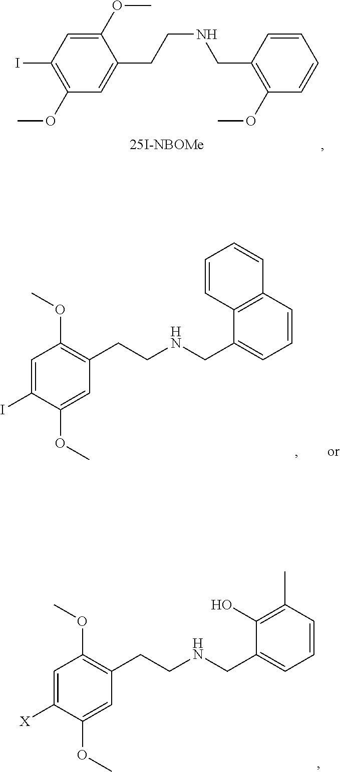

wherein X is CN, Cl, Br or I.

The compounds disclosed herein can be a single isomer or a mixture of isomers or diasteromers. Any chrial center in a compound can be R or S configuration. In some embodiments, the compound is a R-isomer. In some embodiments, the compound is an S-isomer.

In some embodiments, R¹ is OC₁₋₃alkyl. In some embodiments, R² is OC₁₋₃alkyl. In some embodiments, R⁴ is OC₁₋₃alkyl. In some embodiments, R⁶ is OC₁₋₃alkyl. In some embodiments, OC₁₋₃alkyl is OMe.

In some embodiments, the compound include at least one of the following:
(a) R¹ and R⁴ are each independently OC₁₋₃alkyl;
(b) R¹ and R⁵ are each independently OC₁₋₃alkyl; and
(c) R² and R⁵ are each independently OC₁₋₃alkyl.

In some embodiments, R¹ and R⁴ are each independently OC₁₋₃alkyl; R³ is NO₂, haloC₁₋₆alkyl, or C₁₋₆alkyl.

In some embodiments, two adjacent groups (e.g. R¹ and R², R³ and R⁴, R² and R³, R¹ and R⁴, R⁴ and R⁵) of the phenyl ring may link up to form a ring. In some embodiments, R¹ and L¹ link up to form a ring. Non-limiting examples of substituted phenyl with R¹-R⁵ and compounds containg such moieties are shown below. The wiggle line shows the bond connecting the substituted phenyl with other moieties of the compound and the other moieties can be further replaced with different groups as described below in this patent document. While the exemplified structures show methoxy or other oxygen containing substituents on the substituted phenyl, any groups (e.g. ethoxy, propoxy, alkyl, etc) provided above for one or more of R¹, R², R³, R⁴ and R⁵ can be introduced in place of the methoxy or other exemplified substituents. In some embodiments, two adjacent substituents can link up to form a ring.

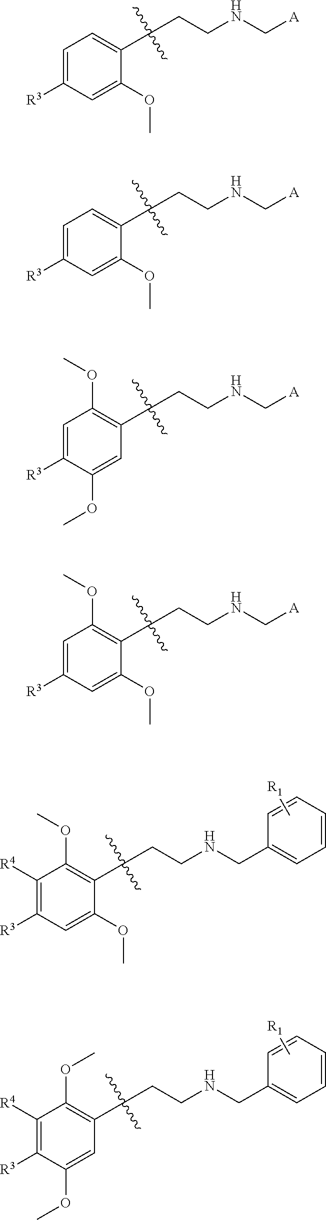

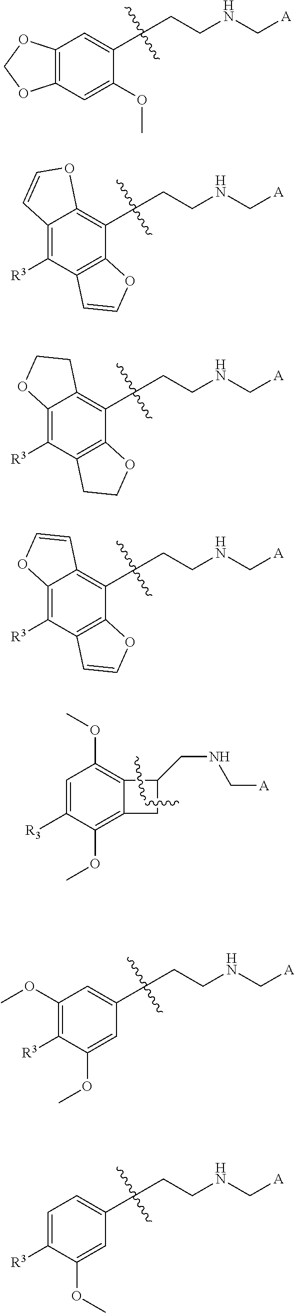

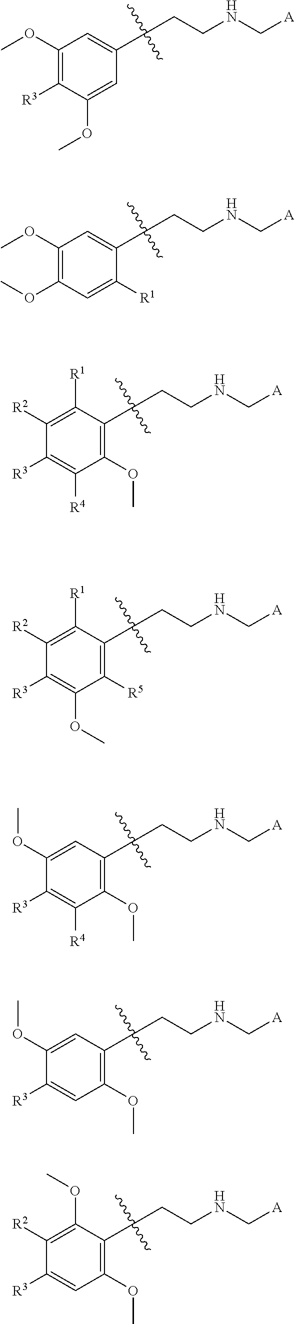

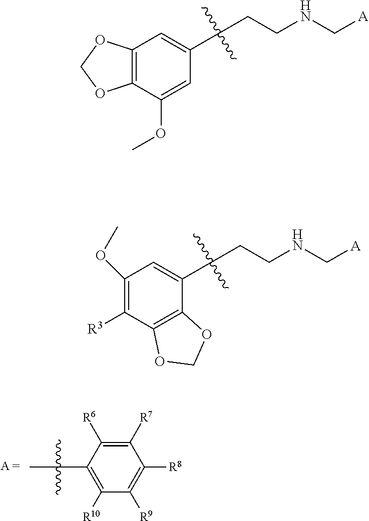

Further examples of the moiety derived from the substituted phenyl containing one or more of R¹, R², R³, R⁴ and R⁵ are shown below. As explained above, the methoxy or other oxygen containing substituents can be replace with other groups (e.g. ethoxy, propoxy, alkyl, etc) defined for one or more of R¹, R², R³, R⁴ and R⁵.

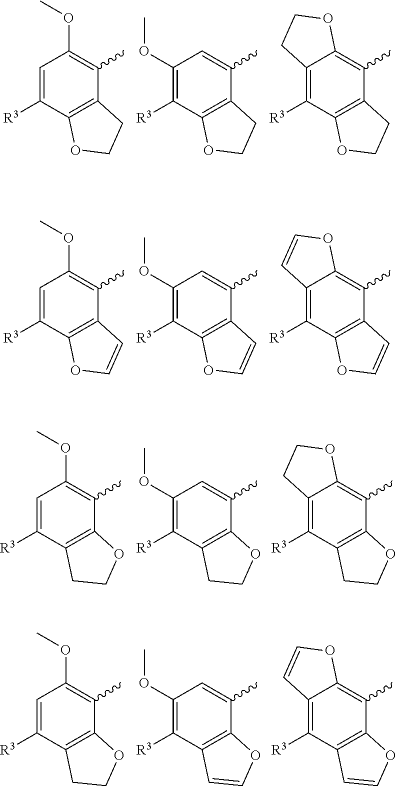

In some embodiments, L¹ is ethylene. In some embodiments, L¹ is an isopropyl.

In some embodiments, L² is C₁₋₃alkylene optionally substituted with C₁₋₄alkyl, C₃₋₆ cycloalkyl, haloC₁₋₄alkyl, deuterium or F. In some embodiments, L² is methylene substituted with methyl, ethyl, fluoro, or deuterium. Non-limiting examples of L² and the attached A ring include the following:

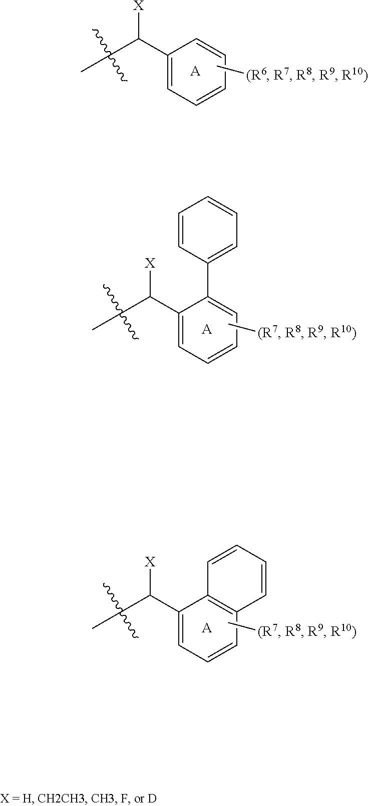

In any embodiments disclosed herein, L² can be methylene. In some embodiments, L² is a bond.

A can be a 4-12 membered saturated or partially saturated monocyclic, bridged or spirocyclic ring. In some embodiments, A is a monocylic ring. In some embodiments, A is phenyl, 5 or 6 membered heteroaryl, 5, 6, 7 or 8 membered cycloalkyl, or 5 or 6 membered heterocycloalkyl.

In some embodiments, two adjacent substituents of A link up and together with A form a bicyclic ring, which can be further substituted with one or more substituents, wherein L² is methylene or a bond. In some embodiments, the two adjacent substituents forming an additional ring are positioned at ring atoms 2 and 3 (ring atom 1 is attached to L²), or ring atoms 3 and 4. For instance, when A is an optionally substituted cyclopentyl, two adjacent substituents at carbons 2 and 3 can link up and together with A form a bicyclic ring as A-1. Alternatively, two adjacent substituents at ring atoms 3 and 4 can link up and together with A form a bicyclic ring as A-2, which can be optionally substituted. In some embodiments, L² is methylene. In some embodiments, L² is a bond.

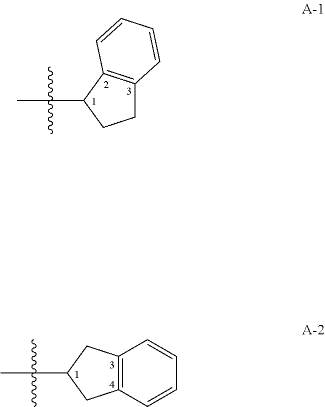 [SMILES: *C1CCc2ccccc21 ; *C1Cc2ccccc2C1]

The moiety A as a monocyclic ring, bicyclic ring or tricyclic ring can be substituted with one or more substituents. Optional substituents include one or more of deuterium, OC₁₋₆alkyl, SC₁₋₆alkyl, CN, OH, halogen, N₃, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}ISO₂C₁₋₆alkyl, C₁₋₆alkylSO₂ (sulfone), S(O)OH, C₁₋₆alkylS(O) (sulfoxide), nitroso, C₁₋₆alkylOSO₂, C₃₋₆cycloalkyl, 3-10 membered heterocycloalkyl, 6-10 membered aryl, 5-10 membered heteroaryl, C₁₋₆alkylene-N(R^{m})₂, and C₁₋₆alkylene-N(R^{m})(COR^{m}). R^{m} each is independently hydrogen or C₁₋₆alkyl or halo-C₁₋₆alkyl.

In some embodiments, Formula I has the following structure. The wiggle line shows the bond connecting the substituted phenyl with other moieties of the compound and the other moieties can be further replaced with different groups as described below in this patent document. One or more of the methoxy groups can be replaced by one or more of R² and R⁵ as defined above.

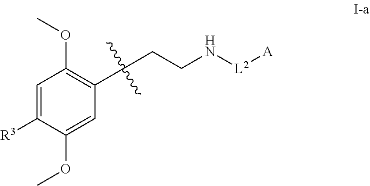

In some embodiments, L² is a bond. In some embodiments, A is A-1 or A-2.

Further examples of bicyclic ring include the following. The ring can be attached to L² at any carbon or at the atom with wiggle line and X can be N, NH, C, CH, CH₂, O, S, CH, CH₂, C═, or C═O as long as the bonding and the overall structure comply with the valancy rule.

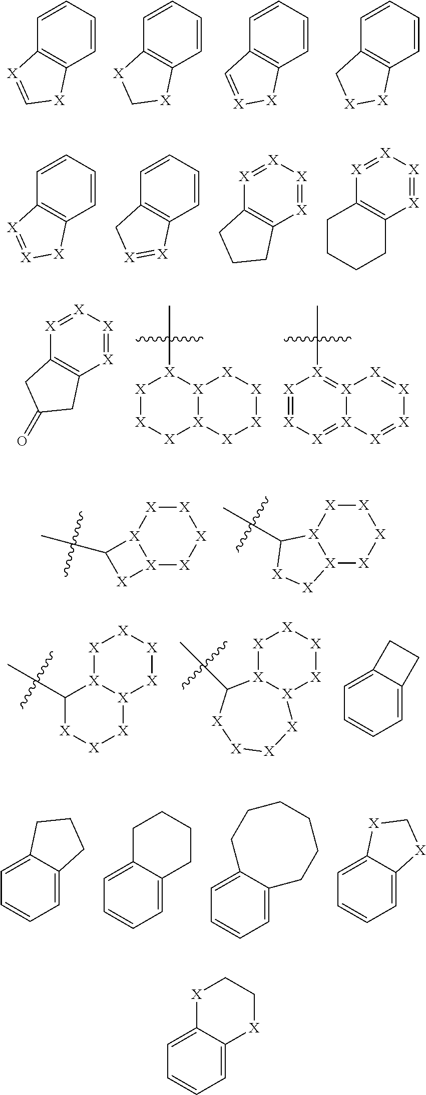

Additional non-limiting examples of optinally substituted bicyclic ring include:

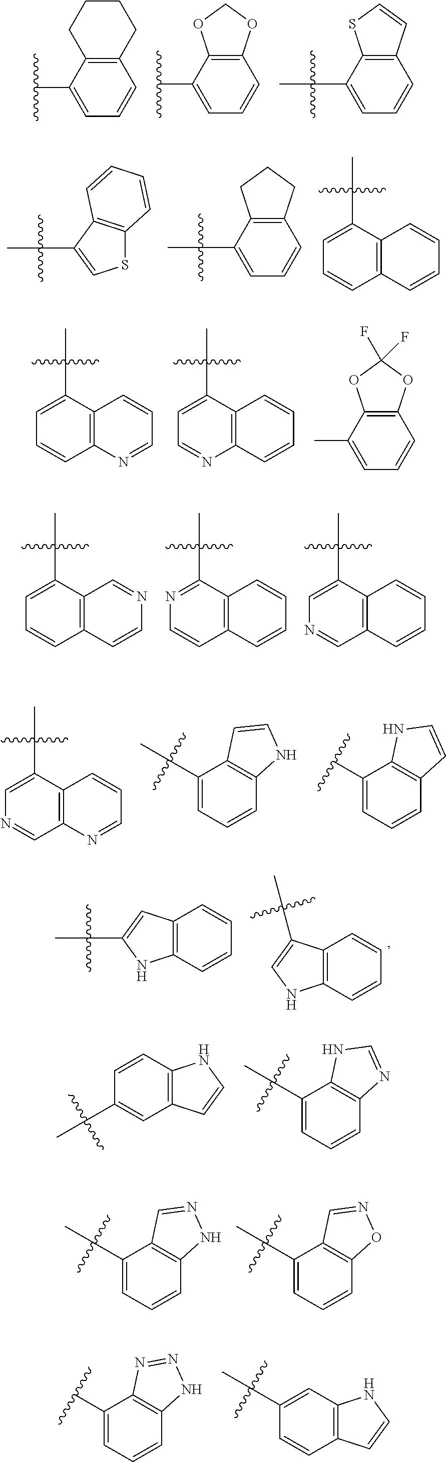

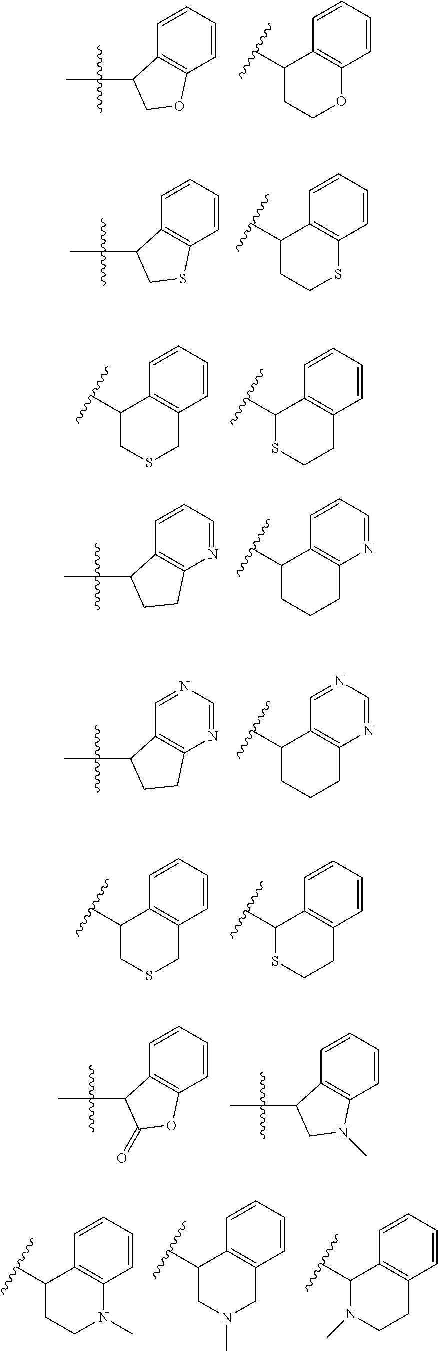

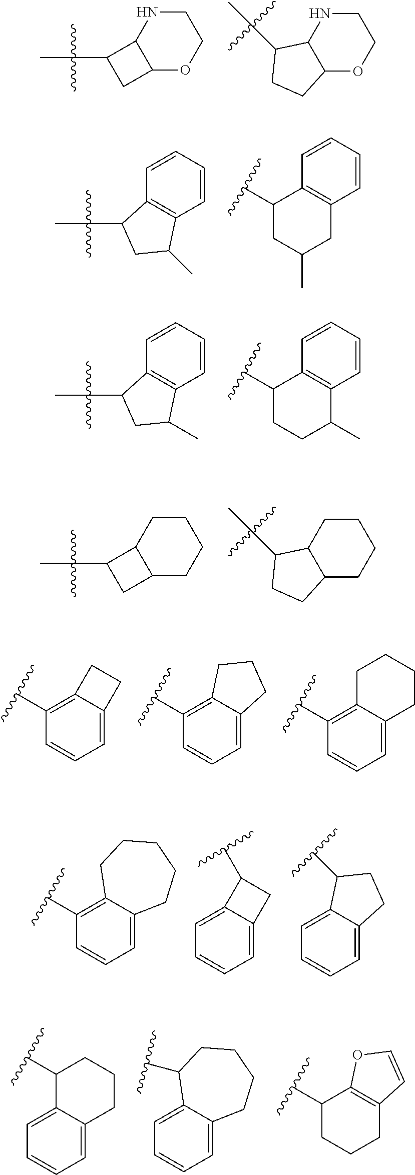

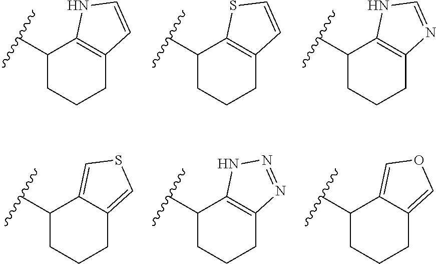

In some embodiments, the optinally substituted bicyclic ring has one of the following structure:

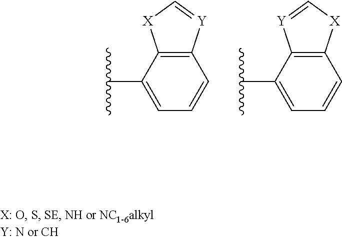

Further non-limiting examples of such bicyclic ring include indanyl, 1,2,3,4-tetrahydronaphthalenyl, benzimidazolyl, benzofuranyl, benzoselenophene, benzothiofuranyl, benzothiophenyl, benzoxazolyl, benzthiazolyl, benztriazolyl, benztetrazolyl, benzisoxazolyl, benzisothiazolyl, benzimidazolinyl, chromanyl, chromenyl, cinnolinyl, indolenyl, indolinyl, indolizinyl, indolyl, 3H-indolyl, indazolyl, isobenzofuranyl, isoindazolyl, isoindolinyl, isoindolyl, isoquinolinyl, methylenedioxyphenyl, naphthyridinyl, naphthalenyl, octahydroisoquinolinyl, phenylnorbomyl, phenylnorbornenyl, quinazolinyl, quinolinyl, 4H-quinolizinyl, quinoxalinyl, tetrahydroisoquinolinyl, and tetrahydroquinolinyl. The bicylic ring can be substituted with one or more substituents selected from OC₁₋₆alkyl, SC₁₋₆alkyl, CN, OH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, and dihydroxyC₁₋₁₀alkyl, C(═NC₁₋₆alkyl)C₁₋₆alkyl, OC(O)N(R^{m})₂, SH, C(O)SRm, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂.

In some embodiments, Formula I has the following structure. The wiggle line shows the bond connecting the substituted phenyl with other moieties of the compound amd the other moieties can be further replaced with different groups as described below in this patent document. One or more of the methoxy groups can be replaced by one or more of of R² and R⁵ as defined above.

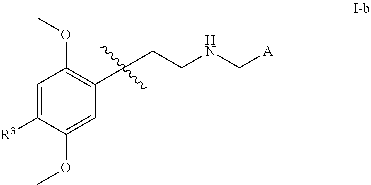

In some embodiments, A is defined as the following

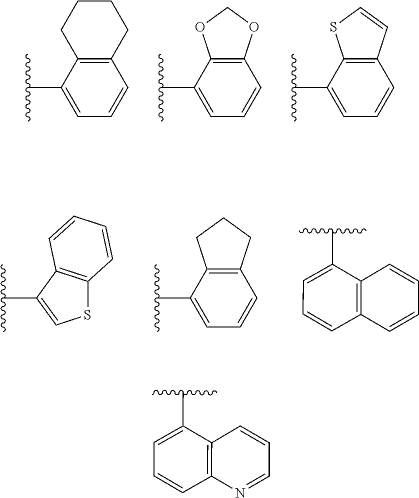

Further examples of tricyclics include the following. X in each instance can be N, NH, C, CH, CH2, O, S, CH, CH₂, C═, or C═O as long as the bonding and the overall structure comply with the valancy rule.

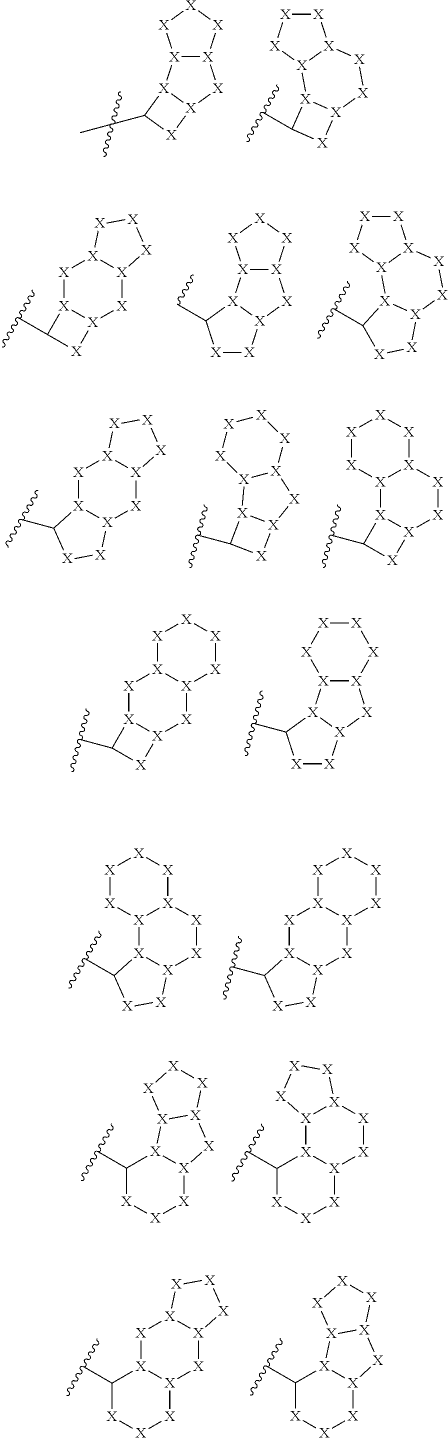

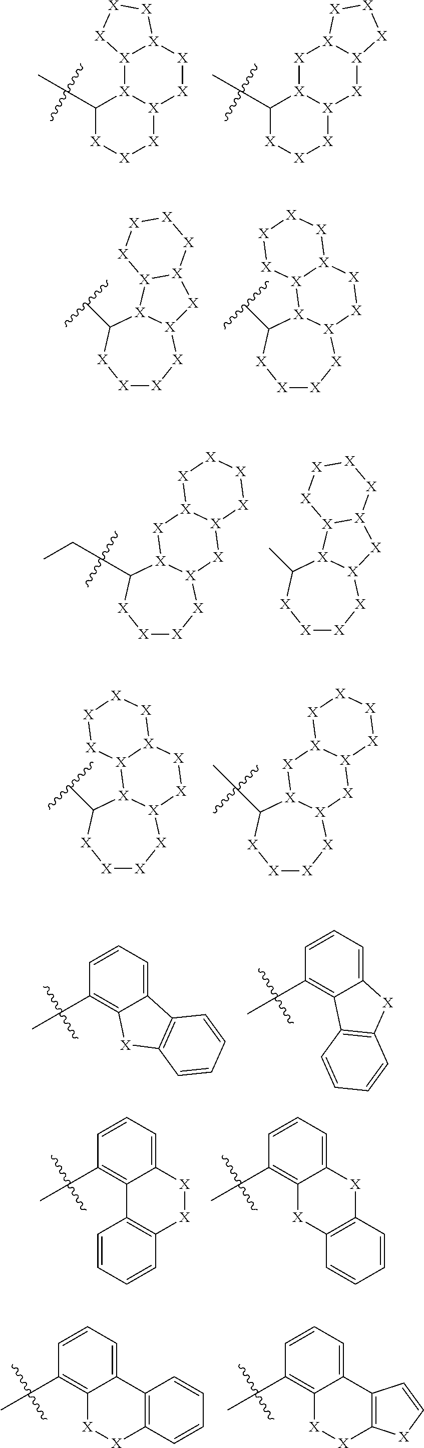

Non-limiting examples of optionally substituted tricyclic rings include the following:

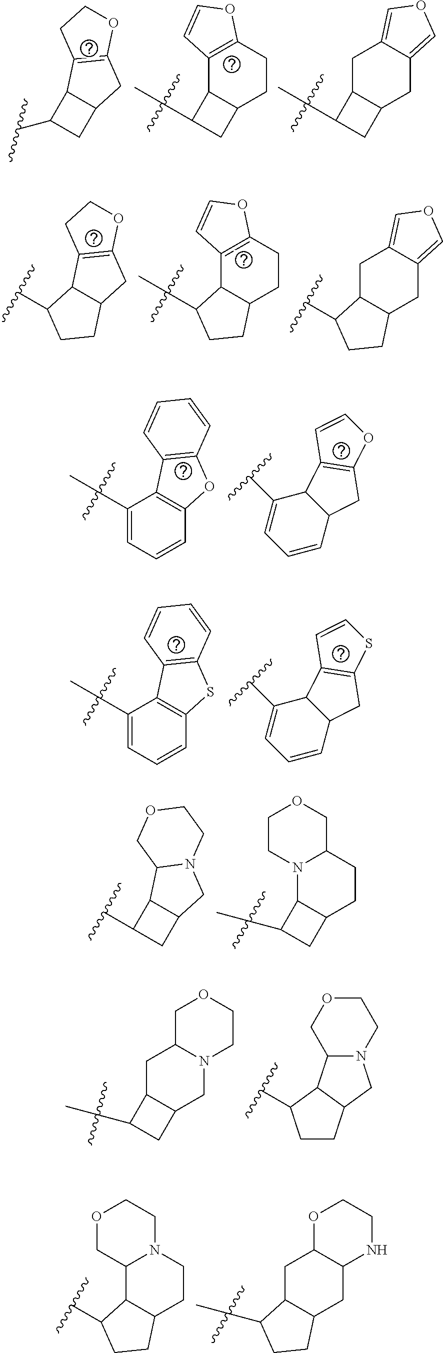

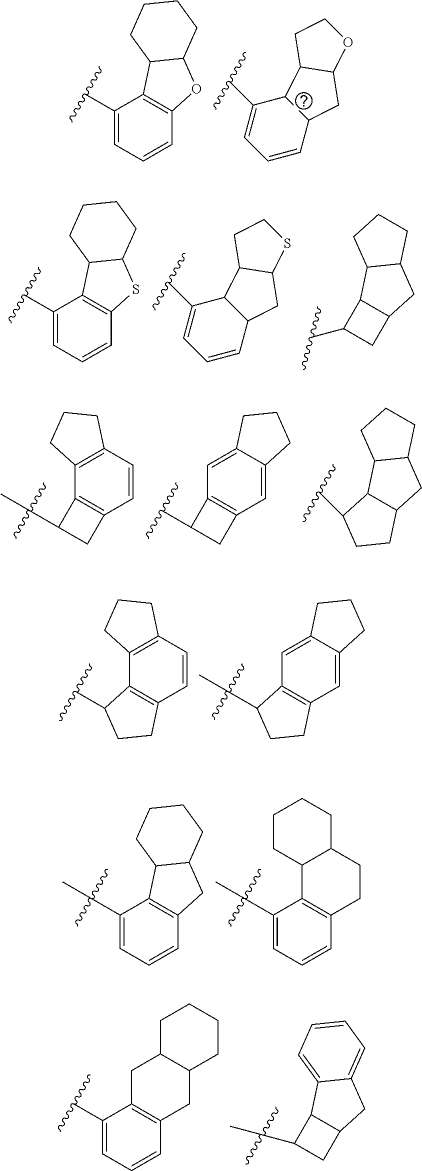

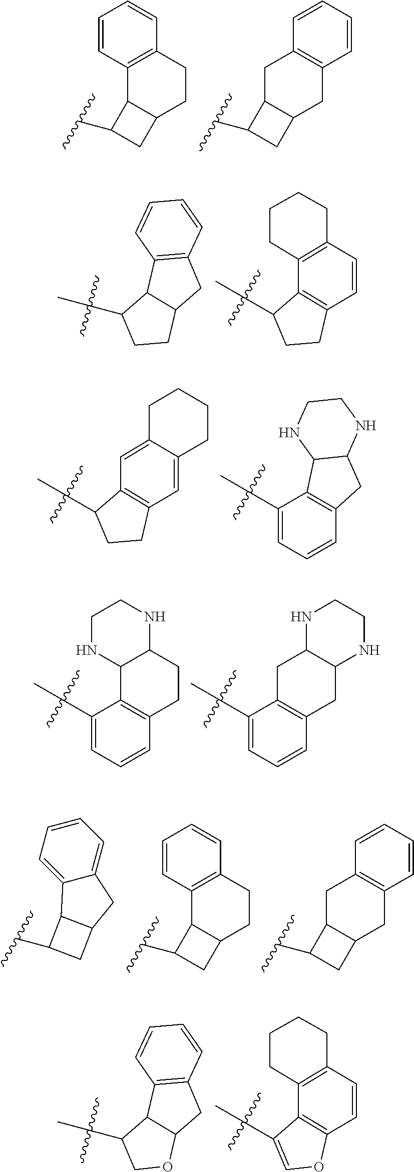

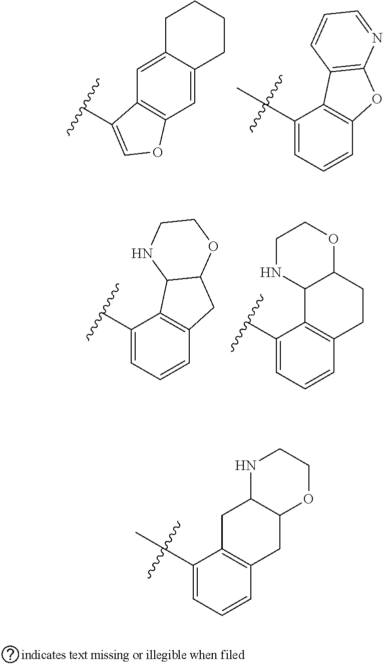

Non-limiting examples of bridged tricyclics include the following:

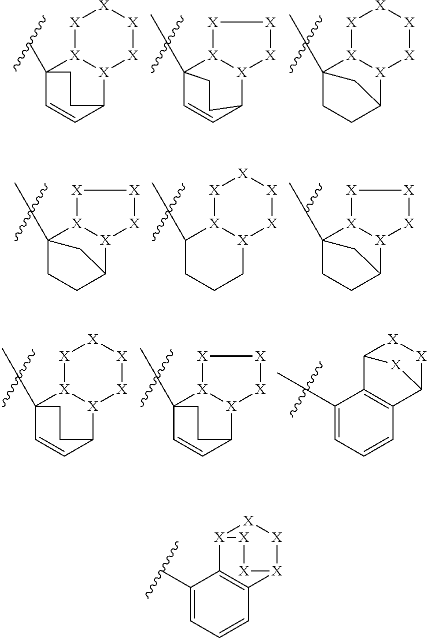

In any ring disclosed herein, it can be attached to L² at any atom as long as the bonding and the overall structure comply with valancy rules.

In some emodiments, A is represented as A-3,

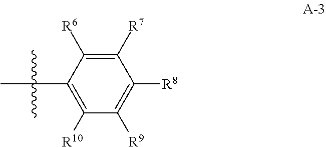

wherein R⁶ and R⁷ are independently selected from the group consisting of H, deuterium, OC₁₋₆alkyl, SC₁₋₆alkyl, CN, OH, halogen, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO2 (sulfone), S(O)OH, C₁₋₆alkylS(O) (sulfoxide), nitroso, C₁₋₆alkylOSO₂, C₃₋₆cycloalkyl, 3-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl₆₋₁₀-membered, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroaryl_{5-10-membered}, OC₁₋₂alkylene-bicycloalkyl_{5-12-membered}, OC₁₋₂alkylene-hetero-bicycloalkyl_{5-12-membered}, C₁₋₂alkylene-C₃₋₆cycloalkyl, C₁₋₂alkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, C₁₋₂alkylene-hetero-bicycloalkyl_{5-12-membered}, wherein the ring moiety of the C₃₋₆cycloalkyl, 3-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl_{6-10-membered}, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroaryl_{5-10-membered}, OC₁₋₂alkylene-bicycloalkyl_{5-12-membered}, OC₁₋₂alkylene-hetero-bicycloalkyl_{5-12-membered}, C₁₋₂alkylene-C₃₋₆cycloalkyl, C₁₋₂alkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, and C₁₋₂alkylene-hetero-bicycloalkyl_{5-12-membered} is optionally substituted with one or more substituents, wherein the optional substituents are selected from deuterium, OC₁₋₆alkyl, SC₁₋₆alkyl, CN, OH, halogen, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂: in some embodiemtns, at least one of R⁶ and R⁷ is not H:

R⁸, R⁹ and R¹⁰ are independently selected from the group consisting of H, deuterium, OC₁₋₆alkyl, SC₁₋₆alkyl, CN, OH, halogen, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxy C₁₋₆alkyl, C(═NC₁₋₆alkyl)C₁₋₆alkyl, OC(O)N(R^{m})₂, SH, C(O)SR″, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC1-6alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, C₃₋₆cycloalkyl, 3-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl_{6-10-membered}, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroaryl_{5-10-membered}, OC₁₋₂alkylene-bicycloalkyl_{5-12-membered}, OC₁₋₂alkylene-hetero-bicycloalkyl_{5-12-membered}, Cizalkylene-C₃₋₆cycloalkyl, Cizalkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, and C₁₋₂alkylene-hetero-bicycloalkyl_{5-12-membered} wherein the ring moiety of the above groups is optionally substituted.

In some emodiments, R⁶ is selected from the group consisting of C₃₋₈ cycloalkyl, 4-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl_{6-10-membered}, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroaryl_{5-10-membered}, OC₁₋₂alkylene-bicycloalkyl_{5-12-membered}, OC₁₋₂alkylene-hetero-bicycloalkyl_{5-12-membered}, C₁₋₂alkylene-C₃₋₆cycloalkyl, C₁₋₂alkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, and C₁₋₂alkylene-hetero-bicycloalkyl_{5-12-membered}, wherein R⁶ is optionally substituted with one or more substituents selected from OC₁₋₆alkyl, SC₁₋₆alkyl, OC₂-6alkenyl, SC₂₋₆alkenyl, OC₂₋₆alkynyl, SC₂₋₆alkynyl, CN, N₃, OH, halogen, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, thiolC₁₋₆alkyl, C₁₋₆alkylene-thioletherC₁₋₆alkyl, aminoC₁₋₆alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂.

In some embodiments, A is A-4, A-5, A-6, A-7 or A-8. In some embodiments, one, two or all of R⁶, R⁷, and R⁸ (if present) are not H.

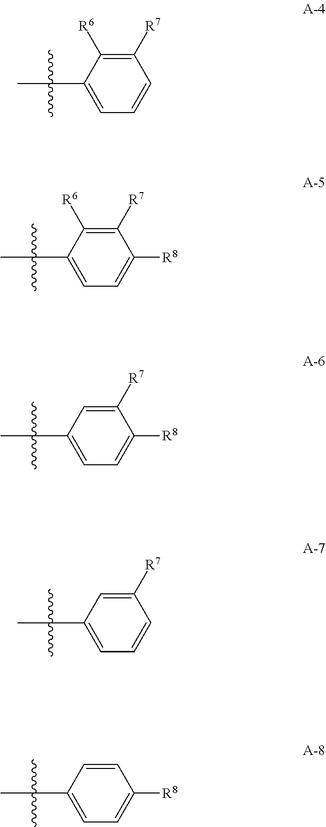

In some embodiments, A is one of the following structures. One or more of R⁶, R⁷, and R⁸ can be optionally further substituted with one or more groups selected from deuterium, OC₁₋₆alkyl, SC₁₋₆alkyl, CN, OH, halogen, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂.

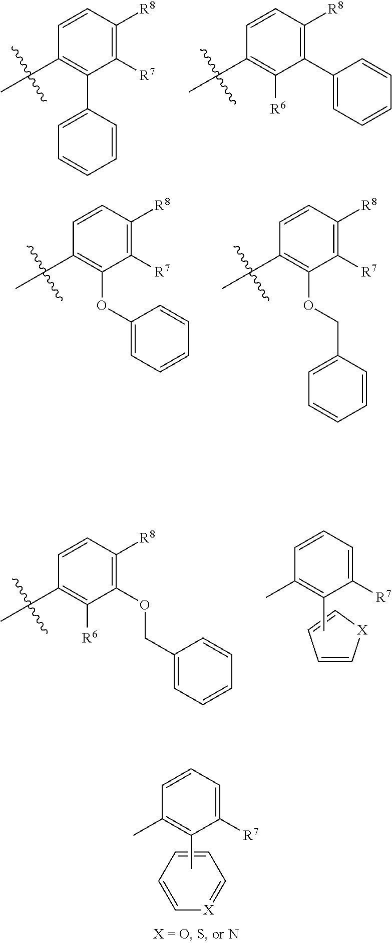

Non-limiting examples of R⁶ as an optionally substituted ring include the following. These R⁶ groups can also be combined with other above described moieties of Formula I (L¹, L², A, etc).

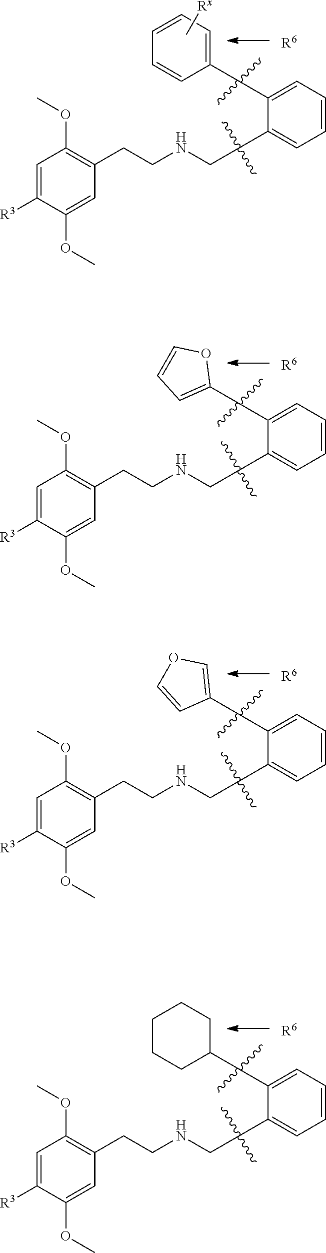

Additional examples of R⁶ are as follows. All these illustrated R⁶ groups are also applicable to R⁷ and R⁸.

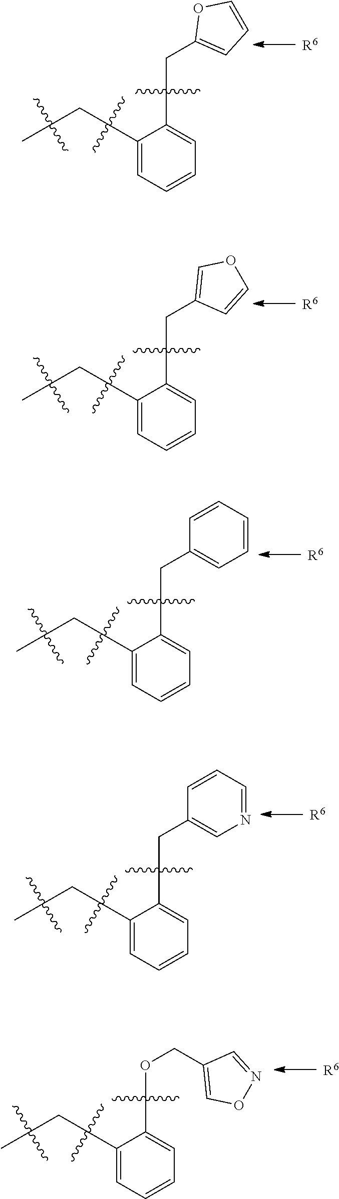

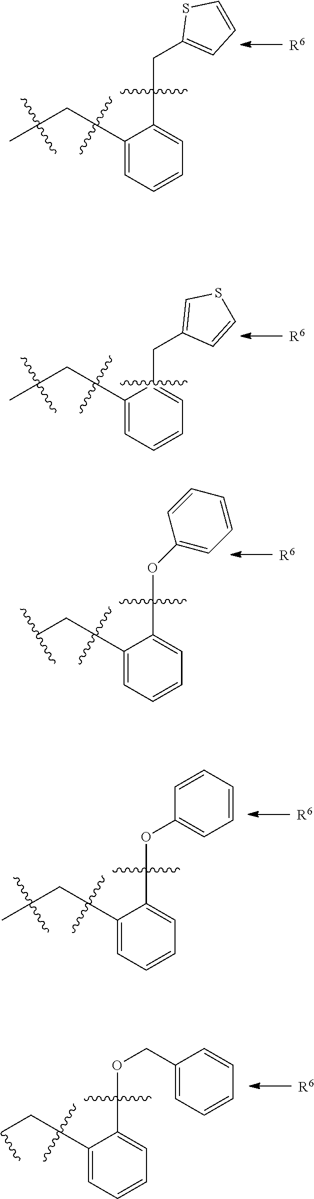

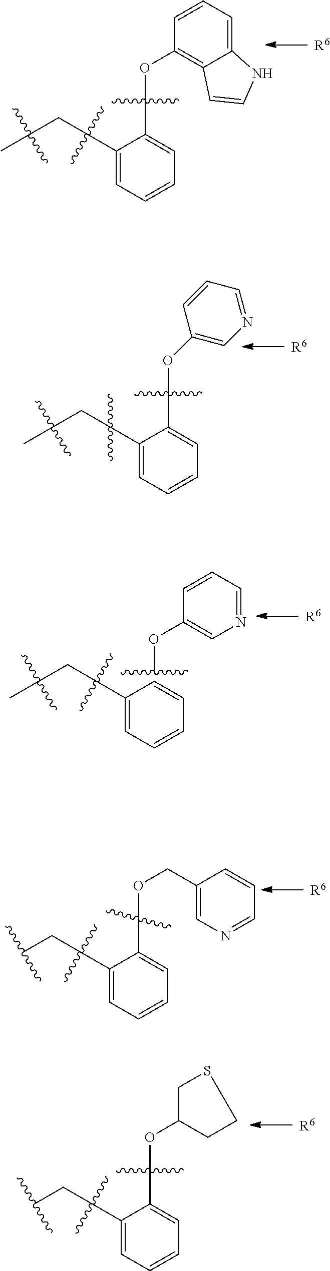

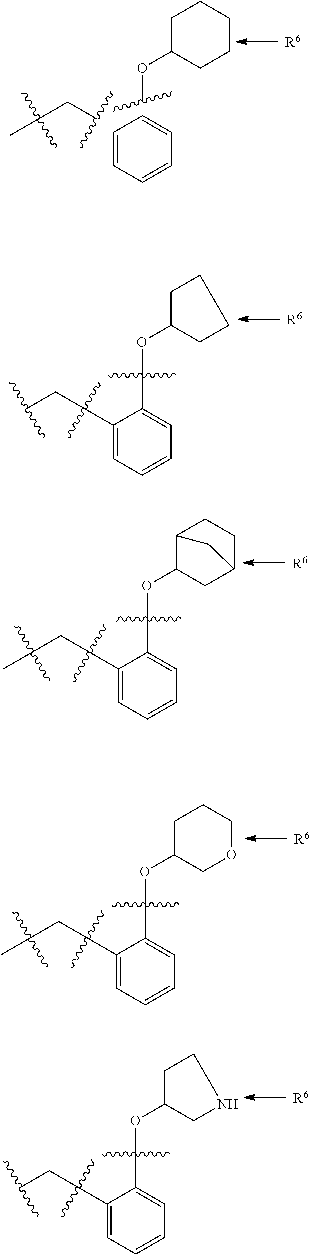

In some embodiments, R⁶ is selected from the group consisting of H, deuterium, OC₁₋₆alkyl, SC₁₋₆alkyl, CN, OH, halogen, N₃, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, and hydroxy C₁₋₆alkyl; and R⁷ is selected from the group consisting of H, deuterium, OC₁₋₆alkyl, SC₁₋₆alkyl, CN, OH, halogen, N(R^{m})₂, C(O)C₁₋₆alkyl, haloC₁₋₆alkyl, haloC₁₋₆alkyleneO, C₁₋₆alkyl, hydroxyC₁₋₆alkyl, thiolC₁₋₆alkyl, C₁₋₆alkylene-thioletherC₁₋₆alkyl, aminoC₁₋₆alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂balkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, C₃₋₇cycloalkyl, 3-10 membered heterocycloalkyl, 6-10 membered aryl, 5-10 membered heteroaryl, 5-12 membered bicycloalkyl, and 5-12 membered hetero-bicycloalkyl, wherein each of the ring is optionally substituted with one or more substituents selected from the group consisting of OC₁₋₆alkyl, SC₁₋₆alkyl, CN, OH, halogen, N₃, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, thiolC₁₋₆alkyl, C₁₋₆alkylene-thioletherC₁₋₆alkyl, aminoC₁₋₆alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂. In some embodiments, R⁷ is an optionally substituted ring selected from C₃₋₆cycloalkyl, C₃₋₆cycloalkyl, 3-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl_{6-10-membered}, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroary_{15-10-membered}, OC₁₋₂alkylene-bicycloalkyl_{5-12-membered}, OC₁₋₂alkylene-hetero-bicycloalkyl_{5-12-membered}, C₁₋₂alkylene-C₃₋₆cycloalkyl, C₁₋₂alkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, and C₁₋₂alkylene-hetero-bicycloalkyl_{5-12-membered}.

Non-limiting examples of compounds containing R⁷ include the following.

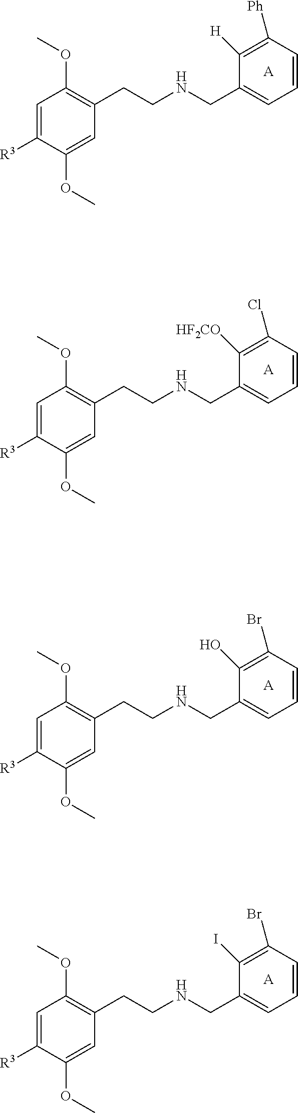

In some embodiments, R⁸ is selected from the group consisting of H, deuterium, OC₁₋₆alkyl, SC₁₋₆alkyl, CN, OH, halogen, N(R^{m})₂, haloC₁₋₆alkyl, haloC₁₋₆alkyleneO, C₁₋₆alkyl, hydroxyC₁₋₆alkyl, C₃₋₆cycloalkyl, 3-10 membered heterocycloalkyl, 6-10 membered aryl, 5-10 membered heteroaryl, 5-12 membered bicycloalkyl, and 5-12 membered hetero-bicycloalkyl, wherein each of the ring is optionally substituted with one or more substituents selected from the group consisting of OC₁₋₆alkyl, SC₁₋₆alkyl, CN, OH, halogen, N(R^{m})₂, C(O)OR^{m} C(O)N(R^{m})₂, C(O)C₁₋₆alkyl, haloC₁₋₆alkyl, haloC₁₋₆alkyleneO, C₁₋₆alkyl, hydroxyC₁₋₆alkyl, thiolC₁₋₆alkyl, C₁₋₆alkylene-thioletherC₁₋₆alkyl, aminoC₁₋₆alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SON(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂.

A bicyclic ring (e.g. a 5-12 membered bicycloalkyl, or a 5-12 membered hetero-bicycloalkyl) can connect to group A or A-3 at any chemically feasible atom of the bicyclic ring. 5-12 membered bicycloalkyl rings and 5-12 membered hetero-bicycloalkyl rings include fused ring, spiro ring and bridged ring. Non-limiting examples include

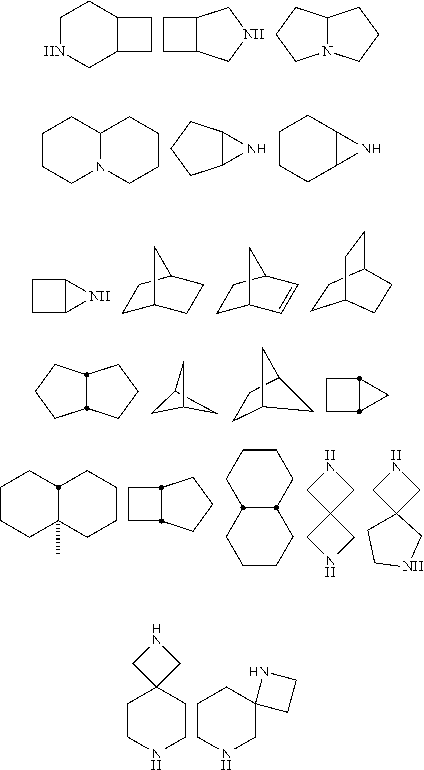

The scope of various ring structures are as defined above. For example, heterocycloalkyl includes 4-8 membered amino heterocylics like azocane, azepane, piperidine, pyrrolidine, azetidine, piperazine, diazepane, as well as morpholine, thiomorpholine, oxazepane, and thiooxazepane. These groups can be connected at any position of the ring to any adjacent group or substituent as long as the connection is in compliance with valency rule. Optionally substituted rings include for example N—C₁₋₆alkyl-piperazine, and N—C₁₋₆alkyl-piperidine.

Additional examples of R⁶, R⁷ and R⁸ as an optionally substituted ring include adamantanyl, cyclopropyl, cyclobutyl, cyclopentyl, cyclopentenyl, cyclohexyl, cyclohexenyl, cycloheptyl, cyclooctyl, tetrahydrofuranyl, ferrocenyl, furanyl, furazanyl, imidazolinyl, imidazolyl, norbornyl, norbornenyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, phenyl, piperazinyl, pyrimidinyl, piperonyl, pyranyl, pyrazinyl, pyrazolidinyl, pyrazolinyl, pyrazolyl, pyridazinyl, pyridinyl, pyridyl, pyrimidinyl, pyrrolinyl, 2H-pyrrolyl, pyrrolyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienothiazolyl, thienooxazolyl, thienoimidazolyl, thiophenyl, triazinyl, 1,2,3-triazolyl, 1,2,4-triazolyl, 1,2,5-triazolyl, and 1,3,4-triazolyl, each of which can be substituted with one or more substituents as described above.

In some embodiments, A is a 5-membered heteroaryl, 5-membered cycloalkyl, or 5-membered hetero-cycloalkyl, which include those defined above for heteroaryl and hetero-cycloalkyl (5-membered). Nonlimting examples of 5-membered structure include thiophene, pyrrole, pyrazole, thiazole, furan, imidazole, tetrahydrofuran, pyrrolidine, 2-pyrrolidone. The 5-membered ring can be optionally substituted with one or more substituents selected from the group consisting of OC₁₋₆alkyl, SC₁₋₆alkyl, CN, OH, halogen, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, thiolC₁₋₆alkyl, C₁₋₆alkylene-thioletherC₁₋₆alkyl, aminoC₁₋₆alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, C₃₋₆cycloalkyl, 3-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl_{6-10-membered}, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroaryl_{5-10-membered}, OC₁₋₂alkylene-bicycloalkyl_{5-12-membered}, OC₁₋₂alkylene-hetero-bicycloalkyl_{5-12-membered}, C₁₋₂alkylene-C₃₋₆cycloalkyl, C₁₋₂alkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, and C₁₋₂alkylene-hetero-bicycloalkyl_{5-12-membered} wherein the ring moiety of the above groups is optionally substituted. In some embodiments, the 5-membered A is further substituted with at least a 5-membered heteroaryl or hetero-cycloalkyl.

In some embodiments, the compounds of Formula I are in the form of Formula I-c, wherein the L¹ is ethylene optionally substituted with C₁₋₃ alky, and L² is methylene optionally substituted with C₁₋₃ alky. In some embodiments, R¹ is a C₁₋₄alkoxy, R³ is C₁₋₄alkoxy, halogen (e.g. F, Cl, Br) or an electron withdrawing group (e.g. NO₂, CN, acetyl, sulfonamide), and one or both of R⁴ and R⁵ C₁₋₄alkoxy.

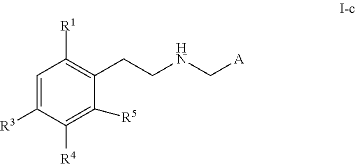

As explained above, C₁₋₄alkoxy can be methoxy, ethoxy, propoxy and butoxy. Preferably, one of R⁴ and R⁵ is C₁₋₄alkoxy and the other is hydrogen. In some embodiments, R⁴ and R⁵ may link up to form a ring (e.g. methylene-dioxy/O—CH₂O). In some embodiments, R³ is C₁₋₄alkoxy (e.g. MeO) or NO₂, R⁴ is C₁₋₄alkoxy (e.g. MeO) and R⁵ is H. In some embodiments, R³ is C₁₋₄alkoxy (e.g. MeO) or NO₂, R⁴ is C₁₋₄alkoxy (e.g. MeO) and R⁵ is H. In some embodiments, R³ and R⁴ link up to form methylene-dioxy and R⁵ is H. In some embodiments, R³ is halogen (e.g. Br), R⁴ is H and R⁵ is C₁₋₄alkoxy (e.g. MeO).

In some embodiments of Formula I-c, A is a substituted phenyl (shown below) or substituted 5 or 6-membered heteroaryl. In some embodiments of Formula I-c, the ethylene moiety (L¹) is substituted with a C₁₋₃alkyl.

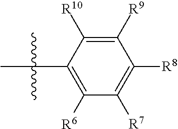

In some embodiments, A is a substituted phenyl, and R⁸, R⁹ and R¹⁰ are each a H. In some embodiments, one or both of R⁶ and R⁷ are independently selected from OH, halogen, C₁₋₁₀alkyl, haloC₁₋₁₀alkyl, S—C₁₋₁₀alkyl, C₁₋₁₀alkoxy, C₃₋₁₀ cycloalkyl, 3-10 membered heterocycloalkyl, phenoxy, benzyloxy, phenyl, 5- or 6-membered heteroaryl, each of which is optionally substituted (e.g. with OH, C₁₋₄alkyl, halogen, or C₁₋₄alkoxy). Alternatively, R⁶ and R⁷ link up and together with A form a fused bicyclic ring (naphthyl). In some embodiments, R⁶ is an electron withdrawing group (e.g. OCF₂H, OCF₃, CHF₂, CF₃, CN or NO₂), and R⁷, R⁸, R⁹ and R¹⁰ are each a H. In some embodiments, R⁶ is C₄₋₁₀alkyl, haloC₄₋₁₀alkyl, S—C₄₋₁₀alkyl, C₄₋₁₀alkoxy, C₃₋₁₀ cycloalkyl, 3-10 membered heterocycloalkyl, phenoxy, benzyloxy, phenyl, 5- or 6-membered heteroaryl, each of which is optionally substituted (e.g. with OH, C₁₋₄alkyl, halogen, or C₁₋₄alkoxy). In some embodiments, R⁷, R⁸, R⁹ and R¹⁰ are each a H.

In some embodiments of Formula I-c, A is a substituted 5 or 6-membered heteroaryl, which is optionally substituted (e.g. with OH, halogen, C₁₋₄alkyl, C₁₋₄alkoxy, benzyloxy, phenyl, 5- or 6-membered heteroaryl). The scope of 5 or 6-membered heteroaryl is as disclosed above, including for example, pyrazole, pyrrole, thiophene, and pyrimidine. In some embodiments, A is a substituted 5-membered heteroaryl, which is further substituted with a phenyl, preferably at a position ortho to L² (e.g. methylene). The phenyl can also be further substituted, for example with OH, C₁₋₄alkyl, halogen, or C₁₋₄alkoxy).

In any embodiment disclosed herein, a substituent can be optionally substituted with for example one or more of deuterium, OC₁₋₆alkyl, SC₁₋₆alkyl, CN, OH, halogen, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, C₃₋₆cycloalkyl, 3-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl_{6-10-membered}, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroaryl_{5-10-membered}, OC₁₋₂alkylene-bicycloalkyl_{5-12-membered}, OC₁₋₂alkylene-hetero-bicycloalkyl_{5-12-membered}, C₁₋₂alkylene-C₃₋₆cycloalkyl, C₁₋₂alkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, and C₁₋₂alkylene-hetero-bicycloalkyl_{5-12-membered}.

### Pharmaceutical Composition and Kit

Another aspect of the patent specification provides a pharmaceutical composition comprising a compound of Formula I or a pharmaceutically acceptable salt thereof disclosed herein and a pharmaceutically acceptable carrier, excipient, or diluent. Compounds described in this patent specification may be formulated by any method well known in the art and may be prepared for administration by any route, including, without limitation, parenteral, peroral, sublingual, buccal, intrathecal, transdermal, topical, subcutaneous, intramuscular, intraperitoneal, intranasal, intratracheal, or intrarectal₂

Nonlimiting examples of pharmaceutically acceptable carriers include physiologically acceptable surface active agents, glidants, plasticizers, diluents, excipients, smoothing agents, suspension agents, complexing agents, film forming substances, and coating assistants. Preservatives, stabilizers, dyes, sweeteners, fragrances, flavoring agents, and the like may be provided in the pharmaceutical composition. For example, sodium benzoate, ascorbic acid and esters of p-hydroxy benzoic acid may be added as preservatives. In addition, antioxidants and suspending agents may be used. In various embodiments, alcohols, esters, sulfated aliphatic alcohols, and the like may be used as surface active agents. Suitable exemplary binders include crystalline cellulose, sucrose, D-mannitol, dextrin, hydroxypropylcellulose, hydroxypropylmethylcellulose, polyvinylpyrrolidone, and the like. Suitable exemplary disintegrants include starch, carboxymethylcellulose, calcium carboxymethylcellulose, croscarmellose sodium, sodium carboxymethylstarch, and the like. Suitable exemplary solvents or dispersion media include water, alcohol (for example, ethanol), polyols (for example, glycerol, propylene glycol, and polyethylene glycol, sesame oil, corn oil, and the like), and suitable mixtures thereof that are physiologically compatible. Suitable exemplary solubilizing agents include polyethylene glycol, propylene glycol, D-mannitol, benzylbenzoate, cyclodextrins, ethanol, trisaminomethane, cholesterol, triethanolamine, sodium carbonate, sodium citrate, and the like. Suitable exemplary suspending agents include surfactants such as stearyltriethanolamine, sodium laurylsulfate, laurylaminopropionic acid, lecithin, benzalkonium chloride, benzethonium chloride, glycerin monostearate, coconut oil, olive oil, sesame oil, peanut oil, soya and the like; and hydrophilic polymers such as polyvinyl alcohol, polyvinylpyrrolidone, sodium carboxymethylcellulose, methylcellulose, hydroxymethylcellulose, hydroxyethylcellulose, hydroxypropylcellulose, and the like. Suitable exemplary isotonic agent includes sodium chloride, glycerin, D-mannose, and the like. Suitable exemplary buffer agents include buffer solutions of salts, such as phosphate, acetates, carbonates, and citrates. Suitable exemplary soothing agents include benzyl alcohol, and the like. Suitable exemplary antiseptic substances include para-oxy benzoic acid esters, benzethonium chloride, benzalkonium chloride, chlorobutanol, benzyl alcohol, phenethyl alcohol, dehydroacetic acid, sorbic acid, and the like. Suitable exemplary antioxidants include sulfite salts, ascorbic acid, and the like. Suitable exemplary sealers include, but are not limited to HPMC (or hypromellose), HPC, PEG and combinations thereof. Suitable exemplary lubricants include magnesium stearate, calcium stearate, talc, colloidal silica, hardened oil and the like.

In further exemplary embodiments for solid preparations, carriers or excipients include diluents, lubricants, binders, and disintegrants. In exemplary embodiments for liquid preparations, carriers include solvents, solubilizing agents, suspending agents, isotonic agents, buffer agents, soothing agents, and the like. Acceptable additional carriers or diluents for therapeutic use and the general procedures for the preparation of pharmaceutical compositions are well known in the pharmaceutical art, and are described, for example, in Remington's Pharmaceutical Sciences, 18th Ed., Mack Publishing Co., Easton, PA (1990), which is incorporated herein by reference in its entirety.

The compound of Formula I may also be in a pharmaceutically acceptable salt form. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt, wherein the counterion include, for example, chloride, bromide, iodide, —O-alkyl, toluenesulfonate, methylsulfonate, sulfonate, phosphate, or carboxy late (such as benzoate, succinate, acetate, glycolate, maleate, malate, citrate, tartrate, ascorbate, benzoate, cinnamoate, mandeloate, benzyloate, and diphenylacetate).

A related aspect provides a kit, which includes a compound of Formula I or a pharmaceutically acceptable salt thereof, or a pharmaceutical composition thereof and an instruction for treating or preventing certain diseases or conditions. In some embodiments, the kit further includes an additional agent.

Non-limiting examples of additional agents include antidepressants (e.g., SSRIs, SNRIs, tricyclic antidepressants, tetracyclic antidepressants, buproprion, ketamine, esketamine, antidepressants, serotonin antagonist and reuptake inhibitors, serotonin modulator and stimulators, monoamine oxidase inhibitors, 5-HTIA agonists, lithium, 5-HT_{2A} agonists (like psilocybin, psilocin, and LSD), antipsychotics, mGlu receptor agonists or antagonists, dextromethorphan (DXM), products containing DXM and quinidine in combination (e.g., Nuedexta®), anxiolytics (benzodiazepines, 5-HT_{1A} agonists, beta-blockers), anticonvulsants (GABA_{A} allosteric modulators, GABA_{A} agonists, calcium channel blockers, voltage-gated sodium channel blockers, glutamate receptor antagonists, glutamate receptor allosteric modulators, GABA transaminase inhibitors, carbamates, carboxamides, valproates, vigabatrin, progabide, tiagabine, topiramate, hydrantoins, oxazolidinediones, beclamide, racetams, succinimides, sulfonamides, triazines), mood stabilizers (e.g., lamotrigine, lithium, valproic acid, divalproex sodium, carbamazepine), pimavanserin, dopamine agonists (e.g., L-DOPA, pramipexole, ropinirole, apomorphine, rotigotine), stimulants, ADHD medications, weight loss medications, antimigraine medications (e.g., triptans, methysergide, ergotamine, naproxen, caffeine, dichloralphenazone, isometheptene), hypnotics/sedatives/sleep aids (GHB, benzodiazepines, zolpidem and other non-benzodiazepine Z drugs, melatonin receptor agonists, antihistamines, barbiturates, orexin antagonists, GABA_{B} receptor modulators, alpha2 adrenergic receptor agonists), amantadine, memantine, acetylcholinesterase inhibitors, cannabinoids, trazadone, nefazodone, and AMPAkines.

Further non-limiting examples of additional agents include analgesics (NSAIDs, opioids, opiates, acetaminophen, steroids, local anesthetics), anti-inflammatory agents (COX-2 inhibitors, NSAIDs, steroids, cannabinoids, immune selective anti-inflammatory derivatives, antileukotrienes), anti-hypertensives (beta-blockers, calcium channel blockers, ACE inhibitors, angiotensin II receptor antagonists, alpha2 receptor agonists, alpha1 receptor antagonists, diuretics) statins, steroids, immunosuppressives (e.g., antimetabolites, macrolides, IMiDs, IL-1 receptor antagonists, mTOR inhibitors, etc), anti-inflammatory and arthritis medications (e.g., tofacitinib, baricitinib, secukinumab), and muscle relaxants.

### \Method of Treating Diseases

Another aspect of the patent specification provides for methods for treating a disease or condition, comprising administering to a subject in need thereof the compound of formula (I), a pharmaceutically acceptable salt thereof, or a corresponding pharmaceutical composition disclosed herein.

The compounds of this patent specification can selectively activate β-arrestin-dependent pathways with specificity over G protein-dependent pathways in relation to other 5-HT_{2A} ligands. It has been discovered that potencies for activating the Gq/11 pathway may correlate best with the hallucinogneic activity of 5-HT_{2A} agonists. G protein-dependent partial agonists activate G protein-dependent pathways to a lesser extent than known hallucinogenic 5-HT_{2A} agonists. In doing so, they are less likely to induce undesirable side effects such as visual hallucinations, delusions, psychosis, and anxiety. Although certain existing 5-HT_{2A} agonists (e.g., lisuride) activate the G protein-dependent pathway with partial efficacy and do not induce hallucinogenic effects, those compounds activate a variety of monoaminergic receptors in a non-selective-manner, which reduces their therapeutic utility and can result in severe or intolerable side-effects. In addition, the selectivity of the compounds disclosed herein for 5-HT_{2A} over 5-HT_{2B} is a major advantage as 5-HT_{2B} agonism is associated with drug-induced cardiac toxicity. Obtaining this degree of selectivity has previously been challenging due to the high degree of sequence homology between 5-HT₂ receptor subtypes.

In some embodiments, the disease or condition is a psychiatric or neurological disease or condition. Non-limiting examples of psychiatric or neurological diseases or conditions include schizophrenia, psychosis, depression, post-traumatic stress disorder, agitation, sexual dysfunction, anxiety, dementias, neurodegenerative diseases, pseudobulbar affect, cluster headache, headache, migraine, pain, neuropathic pain, chronic pain, complex regional pain syndrome, fibromalgyia, drug dependence, drug addiction, alcoholism, hallucinations, delusions, insomnia, epilepsies, bipolar disorder, tinnitus, anorexia, or Parkinson's disease. In some embodiments, the method does not induce hallucinogenic response in the subject or induces a mild hallucinogenic or reduced intensity effects.

Additional examples of diseases or conditions treatable or preventable with the method disclosed herein include a psychiatric or neurological disease or condition or sign or symptom selected from the group consisting of attention deficient disorder, attention deficiet hyperactivity disorder (ADHD), adult attention-deficiet/hyperactivity disorder (AADD, adult ADHD), learning disorders, neurocognitive disorders, Tic disorders, autism spectrum disorder, Tourette's disorder, schizophrenia, negative symptoms of schizophrenia, cognitive symptoms of schizophrenia, substance/medication-induced psychotic disorder, psychotic disorder due to another medical condition, brief psychotic disorder, schizophreniform disorder, schizoaffective disorder, disruptive mood dysregulation disorder, depression, post-partum depression, persistant depressive disorder (dysthymia), major depressive episode, major depressive disorder, treatment-resistant depression, post-traumatic stress disorder, reactive attachment disorder, disinhibited social engagement disorder, personality disorders (e.g., general personality disorder, paranoid personality disorder, schizoid personality disorder, borderline personality disorder, histrionic personality disorder, narcissistic personality disorder, avoidant personality disorder, dependent personality disorder, obsessive-compulsive personality disorder, antisocial personality disorder, schizotypcal personality disorder), psychopathy, cyclothymic disorder, manic episode, hypomanic episode, bipolar disorder, delusional disorder, obsessive compulsive disorder, hoarding disorder, premenstrual dysphoric disorder, somatic symptom and related disorders (e.g., conversion disorder factitious disorders), intellectual disabilities, communication disorders, motor disorders, catalepsy, catatonia, agitation, hypertension, sleep disorders (e.g., insomnia, sleep apnea, hypersomnolence, narcolepsy, nightmare disorder, sleep-wake disorders, non-rapid eye movement sleep arousal disorders, sleepwalking, sleep terrors, rapid eye movement sleep behavior disorder, substance/medication-induced sleep disorder), sexual dysfunctions (e.g., delayed ejaculation, erectile disorder, female orgasmic disorder, female sexual interest/arousal disorder, genito-pelvic pain/penetration disorder, male-hypoactive sexual desire disorder, premature ejaculation, substance/medication-induced sexual dysfunction), anxiety disorders (e.g., selective mutism, generalized anxiety disorder, panic disorder, panic attack, social anxiety disorder, specific phobias, agoraphobia, separation anxiety, hypochondria, substance/medication induced anxiety disorder), adjustment disorders, body dysmorphic disorder, Trichotillomania, excoriation disorder, substance/medication-induced obsessive-compulsive and related disorder, dementias, neurodegenerative diseases (e.g., mild cognitive impairment, Alzheimer's disease, lewy body dementia, frontotemporal dementia, traumatic brain injury, prion diseases, Huntingtion's disease, Parkinson's disease, chronic traumatic encephalopathy, amyotrophic lateral sclerosis, mixed dementias, vascular dementia, hydrocephalus), seasonal affective disorder, pseudobulbar affect, cluster headache, headaches, migraines, Tension-type headaches, tinnitus, hallucinations, delusions, epilepsies, cyclic vomiting syndrome, cannabinoid hyperemesis, nausea, restless leg syndrome, weight loss or binge eating, anorexia nervosa, bulimia nervosa, alcoholism, nicotine dependence, substance use disorders, non-substance related disorders (e.g., gambling disorder, gaming), oppositional defiant disorder, intermittent explosive disorder, conduct disorder, pyromania, kleptomania, paraphilic disorders, medication induced movement disorders, adverse effects of other medications (e.g., antidepressant discontinuation syndrome, neuroleptic malignant syndrome), autoimmune diseases, acute pain, chronic pain, neuropathic pain, cancer, cough, infections, tinnitus, hearing loss, loss of taste, loss of smell, endocrine diseases and disorders, diabetes, gastrointestinal tract related diseases, urinary tract diseases, blood diseases, cardiovascular disease, inflammatory diseases, arthritis, paralysis, and spinal cord injury.

In some embodiments, the disease and condition include for example autoimmune diseases, blood diseases, cardiovascular disease, hypertension, and inflammatory diseases.

In some embodiments, the disease or condition includes for example paralysis or spinal cord injury.

In some embodiments, the method includes administering to a subject an additional agent. Examples of the additional agents are as described above.

Another aspect of the patent document provides a method of activating one or more of 5-HT2 receptors, monoamine receptors, and other CNS relevant receptors by contacting the target receptor with a compound of Formula I. 5-HT2 receptor subtypes include for example, 5-HT2A receptor, 5-HT2B receptor, and 5-HT2C receptor. Monoamine receptors include, for example, receptors for dopamine, serotonin, norepinephrine, and histamine. Nonlimiting examples of other CNS relevant receptors include sigma-1, sigma-2, acetylcholine receptors, glutamate receptors, glycine receptors, GABA receptors, opioid receptors, purine receptors, orexin receptors, and cation channels. Further examples of targets that can be activated with compounds of Formula I include receptors for epinephrine, adenosine, acetylcholine, GABA, glutamate, glycine, ion channels or transporters, and other 5-HT receptors. In some embodiments, the compound is selected to activate or exert pharmacological action on 5-HT2 receptor subtypes. In some embodiments, the compound is selected to activate one or more of 5-HT2A receptor, 5-HT2B receptor and 5-HT2C receptor. In some embodiments, the compound is selected to selectively activate one or more 5-HT2 receptor subtypes over other receptors including the aforementioned receptors (e.g. Monoamine receptors, CNS relevant receptors, receptors for epinephrine, adenosine, acetylcholine, GABA, glutamate, glycine, ion channels or transporters, and other 5-HT receptors). Contacting the compound with the target receptor may take place in vitro or in vivo.

In some embodiments of any method disclosed herein, a compound of Formula I is selected to selectively exert pharmacological action on or activate 5-HT2 receptors over other pharmacological targets (e.g., receptors). In some embodiments, a compound of Formula I is selected to selectively activate 5-HT2 receptors over other 5-HT receptors. In some embodiments, a compound of Formula I is selected to selectively activate:
(a) 5-HT2A receptor over other 5-HT receptors;
(b) 5-HT2B receptor over other 5-HT receptors;
(c) 5-HT2C receptor over other 5-HT receptors;
(d) 5-HT2A receptor and/or 5-HT2B receptor over other 5-HT2C receptor;
(e) 5-HT2A receptor over 5-HT2B receptor: or
(f) 5-HT2A receptor over 5-HT2B and/or 5-HT2C receptor.

The selectivity of a compound of Formula may be defined in terms of binding affinity or functional selectivity. For instance, the compound may exhibit a higher receptor binding affinity to 5-HT2A and/or 5-HT2C over 5-HT2B by for example 2-20,000 folds, 5-10,000 folds or 5-1,000 folds. The functional selectivity indicates that the compound is capable of exhibiting a stronger maximal response to a target receptor over another receptor with respect to one or more signaling pathways. The functional selectivity may be determined in terms of the difference in potency (e.g. a more potent EC50 by for example 2-10,000 folds, 2-1,000 folds or 5-500 folds) or the difference in Emax (e.g. the difference in Emax between two receptors signaling pathways) as well as combinations of potency and Emax.

The Emax is generally determined in comparison with a reference compound (e.g. 5-HT) and expressed in a percentage value. While the percentage value of Emax may vary depending on the assay and the experimal conditions, the rank order for compounds in comparison with the reference such as a known psychedelics generally holds a consistent trend. The difference in Emax between two different pathways is calculated via Emax (one pathway) —Emax (another pathway). For example, if Emax (one pathway) and Emax (another pathway) are 110% and 20%, respectiviely, their difference is 90%.

In any embodiment of compounds, compositions, kits or methods disclosed herein, a selected compound of Formula I may be characterized by any one, any two, any three or all of the following:
(1) a binding affinity to a target receptor over one or more other receptors by more than 2 folds, more than 3 folds, more than 4 folds, more than 5 folds, more than 10 folds, more than 2-folds, more than 50 folds, more than 100 folds, more than 200 folds, more than 500 folds, more than 1,000 folds, more than 2,000 folds, more than 5,000 folds, more than 10,000 folds, or more than 20,000 folds;
(2) a potency (e.g. EC50) to a target receptor over one or more other receptors by more than 2 folds, more than 3 folds, more than 4 folds, more than 5 folds, more than 10 folds, more than 2-folds, more than 50 folds, more than 100 folds, more than 200 folds, more than 500 folds, more than 1,000 folds, more than 2,000 folds, more than 5,000 folds, more than 10,000 folds, or more than 20,000 folds;
(3) a difference in Emax between pathways (e.g. G protein and arrestin pathways, which can be the same or different) at two different receptors by about 1%, about 2%, about 5%, about 10%, about 15%, about 20%, about 30%, about 50%, about 60%, about 70%, about 80%, about 90%, about 100%, about 110%, about 120%, about 130%, about 140%, about 150%, about 160%, about 180%, or about 200%.
(4) a difference in Emax (biased signaling) between pathways (e.g. G protein and arrestin pathways) at a single receptor by about 1%, about 2%, about 5%, about 10%, about 15%, about 20%, about 30%, about 50%, about 60%, about 70%, about 80%, about 90%, about 100%, about 110%, about 120%, about 130%, about 140%, about 150%, about 160%, about 180%, or about 200%.

In some embodiments of compounds and methods disclose herein, a compound of Formula exhibits a functional selectivity between receptors, independent of relative potency, and is characterized by a difference in Emax ranging from about 1% to about 150%, from about 5% to about 200%, from about 10% to about 150%, from about 10% to about 100%, from about 10% to about 80%, from about 1% to about 50%, from about 1% to about 20%, or from about 1% to about 15% between the two signaling pathways.

In some embodiments of compounds and methods disclose herein, a compound of Formula I selectivity activates 5-HT2A (or G protein and/or arrestin) and/or 5-HT2C over 5-HT2B and is characterized by: a binding affinity ranging from about 2 to about 20,000 fold, about 2 to about 10,000 fold, about 5 to about 10,000 fold, about 3 to about 1,000 fold, or about 5 to about 500 fold for the target (5-HT2A (G protein and/or arrestin) and/or 5-HT2C) over 5-HT2B, a stronger potency (EC50) ranging from about 5 to about 20,000 fold, from about 5 to about 10,000 fold, from about 2 to about 2,000 fold, ranging from about 2 to about 1,000 fold, ranging from about 5 to about 500 fold, or ranging from about 10 to about 100 fold for a response (e.g., G protein and/or arrestin) between two or more receptors, and/or a difference in Emax ranging from about 1% to about 150%, from about 5% to about 200%, from about 10% to about 150%, from about 10% to about 100%, from about 10% to about 80%, from about 1% to about 50%, from about 1% to about 20%, or from about 1% to about 15% between G protein pathway and arrestin pathway. In some embodiments, the Emax is higher for a G protein pathway than for an arrestin pathway. In some embodiments, the Emax is lower for G protein pathway than for arrestin pathway.

In some embodiments of compounds and methods disclose herein, a compound of Formula I selectively activates a given signaling pathway over another signaling pathway, at two different receptors or at a single receptor, may be characterized by the following: the difference in Emax is higher for an arrestin pathway than for a G protein pathway and ranges from about 1% to about 80%, from about 1% to about 50%, from about 1% to about 20%, from about 1% to about 10%, from about 2% to about 50%, from about 5% to about 20%, or from about 5% to about 10%, and the potency (EC50) for an arrestin pathway is greater than for a G protein pathway ranging from about 2 to about 20,000 fold, from about 2 to about 10,000 fold, from about 5 to about 10,000 fold, from about 10 to about 5,000 fold, from about 10 to about 500 fold, from about 20 to about 100 fold.

In some embodiments of compounds and methods disclose herein, a selected compound of Formula I selectively activates a signaling pathway and may be characterized by the following: the difference in Emax is equal to or lower for the arrestin pathway than for a G protein pathway and ranges from about 1% to about 80%, from about 1% to about 50%, from about 1% to about 20%, from about 1% to about 10%, from about 2% to about 50%, from about 5% to about 20%, from about 5% to about 10%, or from about 2% to about 5%, and the potency (EC50) is greater for an arrestin pathway than for a G protein pathway ranging from about 2 to about 20,000 fold, from about 2 to about 10,000 fold, from about 5 to about 10,000 fold, from about 10 to about 5,000 fold, from about 10 to about 500 fold, from about 20 to about 100 fold.

In some embodiments of compounds and methods disclose herein, a selected compound of Formula I induces more than 20%, more than 30%, more than 40%, more than 50%, more than 60%, more than 70%, more than 80%, or more than 90% of response produced by 5-HT for an β-arrestin pathway. In some embodiments, the compound induces about 100% of response produced by 5-HT or induce greater than 100% of response produced by 5-HT for a β-arrestin pathway. In some embodiments, the compound In some embodiments, the compound induces (1) less than 100%, less than 90%, less than 80%, or less than 70%, and (2) greater than 20%, greater than 30%, greater than 40% or greater than 50% of response produced by 5-HT for β-arrestin recruitment.

In some embodiments of compounds and methods disclose herein, a selected compound of Formula I exhibits a G protein pathway partial agonism and is characterized by an Emax for a G protein pathway greater than 10% (or greater than 20%, or greater than 30% or greater than 40%) but less than or equal to 100% (or less than or equal to 80% or less than or equal to 70% or less than or equal to 60%). Therefore in some embodiments, a compound of Formula I can be used to partially agonize a G protein pathway. As explained above, while the Emax value may vary depending on the assay and conditions, the rank order in the context of a identified reference provides sufficient guidance to evaluate the activity of a compound.

In some embodiments of compounds and methods disclose herein, a selected compound of Formula I exhibits G protein partial agonism and is characterized by an Emax for G protein pathway lower than 80% (or lower than 90% or lower than 60%) and the Emax for arrestin pathway is less than, equivalent to, or greater than the Emax for G protein pathways.

In some embodiments of compounds and methods disclose herein, a compound of Formula I selectively activate β-arrestin-dependent pathways over G protein-dependent pathways. The method includes contacting a serotonin 5-HT_{2A} receptor with a compound of Formula I or a pharmaceutically acceptable salt thereof disclosed herein.

5-HT_{2A} agonists that act as partial agonists for G protein-dependent signaling or are β-arrestin-biased have been found to activate the 5-HT_{2A} receptor without inducing the HTR in animal studies. In some embodiments of the methods described in this patent specification, the compound or a pharmaceutically acceptable salt thereof is a partial or full β-arrestin agonist or superagonist (e.g. exceeds or >100% of the response to 5-HT). In some embodiments, the compound or a pharmaceutically acceptable salt thereof is a G protein signaling-preferring partial agonist. In some embodiments, the compounds show β-arrestin bias, with reduced effect on G protein-coupled pathways related to hallucinogenic 5-HT2A ligands. In the latter case, the potency or efficacy of the compound or a pharmaceutically acceptable salt thereof prefers β-arrestin recruitment and supercedes G protein agonism by, for example, greater than or equal to 10-150% difference in Emax. In some embodiments, the compound of Formula I has an Emax of less than 80%, less than 75%, less than 70%, less than 60%, or less than 50% for one or both of β-arrestin dependent pathway and G protein-dependent pathway. In some embodiments, a compound of Formula I is selected to selectively activate a β-arrestin-dependent pathway over a G protein-dependent pathway with a stronger potency by more than 2-fold, more than 5-fold, more than 10-fold, more than 20-fold, more than 50-fold, more than 100-fold, more than 200-fold, more than 500-fold, or more than 1000-fold.

In some embodiments, the compound or pharmaceutically acceptable salt thereof is selective for 5-HT_{2A} over one or both of 5-HT_{2B} and 5-HT_{2C}. The affinity, functional potency or efficacy of the compound or pharmaceutically acceptable salt thereof for 5-HT_{2A} is more than 2-fold, more than 5-fold, more than 10-fold, more than 20-fold, more than 50-fold, more than 100-fold, more than 200-fold, more than 500-fold, or more than 1000-fold higher than the potency or affinity for one or both of 5-HT_{2B} and 5-HT_{2C}.

In some embodiments, the contact between a 5-HT_{2A} receptor and the compound or pharmaceutically acceptable salt thereof occurs in vivo, such as in an animal or in a human.

Another aspect provides a method of alleviating or preventing the psychedelic activity (including the hallucinogenic effects) of another 5-HT_{2A} agonist. The method includes administering to a subject in need a compound of Formula I or a pharmaceutically acceptable salt thereof disclosed herein. The administering step may take place prior to, simultaneously, or subsequent to the administration of the hallucinogenic 5-HT_{2A} agonist. For instance, a subject who has taken a hallucinogen (e.g. a member of the lysergamide, tryptamine, or phenylalkylamine structural classes, etc.) can be treated with the present method to minimize unwanted side-effects. Alternatively, to control the undesirable side-effects of a hallucinogen or to interrupt any psychedelic effect if there is an emergency or an adverse effect occurs (anxiety, panic, confusional state, delusion, psychosis, suicidality, chest pain, hypertensive crisis, or a fire or other event requiring evacuation), a compound of Formula I or a pharmaceutically acceptable salt thereof can be administered prior to, at the same time, or after as a hallucinogenic drug is taken or an emergency/adverse event occurs.

Another aspect provides a method to reduce 5-HT_{2B} agonism induced by a 5-HT_{2A} agonist with 5-HT_{2B} agonist activity, or by any 5-HT_{2B} agonist drug. The method includes administering to a subject in need a compound of Formula I or a pharmaceutically acceptable salt thereof disclosed herein. The administering step may take place prior to, simultaneously, or subsequent to the administration of the 5-HT_{2B} agonist. For instance, a subject who has taken a hallucinogen (e.g. a member of the lysergamide, tryptamine, or phenylalkylamine structural classes, etc.) can be treated with the present method to minimize unwanted side-effects. This may be necessary with microdosing—which involves taking low doses of 5-HT_{2A} agonists.

### Administration Regimen

The compound of Formula I, or a pharmaceutically acceptable salt thereof or a pharmaceutically composition thereof for the methods or kit described herein described herein may be administered to the subject by any suitable means. Non-limiting examples of methods of administration include, among others, (a) administration though oral pathways, which administration includes administration in capsule, tablet, granule, spray, syrup, film, tincture, drops, implant, or other such forms: (b) administration through non-oral pathways such as rectal, vaginal, intraurethral, intraocular, intranasal, or intraauricular, which administration includes administration as an aqueous suspension, an oily preparation or the like or as a drip, spray, suppository, salve, ointment or the like: (c) administration via injection, subcutaneously, intraperitoneally, intravenously, intramuscularly, intradermally, intraorbitally, intracapsularly, intraspinally, intrasternally, or the like, including infusion pump delivery: as well as (d) administration topically: as deemed appropriate by those of skill in the art for bringing the active compound into contact with living tissue.

Advantageously, the compound of Formula I, or a pharmaceutically acceptable salt thereof or a pharmaceutically composition thereof for administrations described above are prepared into dosage forms in a unit dose suited to fit a dose of the active ingredients. Such dosage forms in a unit dose include, for example, tablets, pills, capsules, injections (ampoules), suppositories, etc.

In exemplary embodiments of the pharmaceutical composition of the compound of Formula I, or a pharmaceutically acceptable salt thereof for oral administration, the composition can be a tablet, coated tablet, capsule, caplet, cachet, lozenges, gel capsule, hard gelatin capsule, soft gelatin capsule, troche, dragee, dispersion, powder, granule, pill, liquid, an aqueous or non-aqueous liquid suspension, an oil-in-liquid or oil-in-water emulsion, including sustained release formulations that are known in the art. For pediatric and geriatric applications, suspensions, syrups and chewable tablets are especially suitable.

The therapeutically effective amount (dosage) of the compound of Formula I, or a pharmaceutically acceptable salt thereof required will depend on the route of administration, the species (human or animal), and the physical characteristics of the particular subject or subject being treated. The dose can be tailored to achieve a desired effect, but will depend on such factors as weight, diet, concurrent medication and other factors which those skilled in the medical arts will recognize. More specifically, a therapeutically effective amount means an amount of compound effective to prevent, alleviate or ameliorate symptoms of disease or prolong the survival of the subject or animal being treated. Determination of a therapeutically effective amount is well within the capability of those skilled in the art, especially in light of the detailed disclosure provided herein.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be about 10 μg/kg to about 100 mg/kg body weight, preferably about 100 μg/kg to about 10 mg/kg body weight. Alternatively, dosages may be based and calculated upon the surface area of the animal, as understood by those of skill in the art.

The exact formulation, route of administration and dosage for the pharmaceutical compositions can be chosen by the individual physician in view of the subject's condition. (see e.g., Fingl et al. 1975, in “The Pharmacological Basis of Therapeutics”, which is hereby incorporated herein by reference in its entirety, with particular reference to Ch. 1, p. 1). In some embodiments, the dose range of the compound of Formula I or a pharmaceutically acceptable salt thereof administered to the subject or subject can be from about 0.5 to about 1000 mg/kg of their body weight. The dosage may be a single one or a series of two or more given in the course of one or more days, as is needed by the subject. In instances where human dosages for compounds have been established for at least some conditions, those same dosages, or dosages that are about 0.1% to about 500%, more preferably about 25% to about 250% of the established human dosage may be used.

It should be noted that the attending physician would know how to and when to terminate, interrupt, or adjust administration due to side-effects, toxicity or organ dysfunctions. Conversely, the attending physician would also know to adjust treatment to higher levels if the clinical response was not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency will also vary according to the age, body weight, and response of the individual subject. A program comparable to that discussed above may also be used in veterinary medicine.

Although the exact dosage will be determined on a drug-by-drug basis, in most cases, some generalizations regarding the dosage can be made. The daily dosage regimen for an adult human subject may be, for example, a peroral dose of about 0.01 mg to 2000 mg of the active ingredient, preferably from about 0.01 mg to about 500 mg. In other embodiments, an intravenous, subcutaneous, or intramuscular dose of the active ingredient of about 0.01 mg to about 100 mg, preferably about 0.01 mg to about 60 mg is used. In cases of administration of a pharmaceutically acceptable salt, dosages may be calculated as the freebase. In some embodiments, the composition is administered 1 to 4 times per day. Alternatively, a compound of Formula I or a pharmaceutically acceptable salt thereof may be administered by continuous intravenous infusion, preferably at a dose of up to about 1000 mg per day. As will be understood by those of skill in the art, in certain situations it may be necessary to administer a compound of Formula I or a pharmaceutically acceptable salt thereof disclosed herein in amounts that exceed, or even far exceed, the above-stated, preferred dosage range in order to effectively and aggressively treat particularly intractable diseases or conditions. In some embodiments, a compound of Formula I or a pharmaceutically acceptable salt thereof will be administered for a period of continuous therapy, for example for a week or more, or for months or years.

In some embodiments, a compound of Formula I or a pharmaceutically acceptable salt thereof is formulated into a dosage form for release for a period of 1 to 12, typically 3 to 12 hours, more typically 6-12 hours after administration. In some embodiments, the oral pharmaceutical compositions described herein may be administered in single or divided doses, from one to four times a day. The oral dosage forms may be conveniently presented in unit dosage forms and prepared by any methods well known to those skilled in the art of pharmacy.

A compound of Formula I or a pharmaceutically acceptable salt thereof can be evaluated for efficacy and toxicity using known methods. For example, the toxicology of the compound may be established by determining in vitro toxicity towards a cell line, such as a mammalian, and preferably human, cell line. The results of such studies are often predictive of toxicity in animals, such as mammals, or more specifically, humans. Alternatively, the toxicity may be determined in an animal model (such as mice, rats, rabbits, or monkeys) using known methods. The efficacy of a particular compound may be established using several recognized methods, such as in vitro methods, animal models, or human clinical trials. Recognized in vitro models exist for nearly every class of condition. Similarly, acceptable animal models may be used to establish the efficacy of chemicals to treat such conditions. When selecting a model to determine efficacy, the skilled artisan can be guided by the state of the art to choose an appropriate model, dose, and route of administration, and dosing regime. Of course, human clinical trials can also be used to determine the efficacy of a compound of Formula I or a pharmaceutically acceptable salt thereof in humans.

A compound of Formula I or a pharmaceutically acceptable salt thereof may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of Formula I or a pharmaceutically acceptable salt thereof formulated in a compatible pharmaceutical carrier may also be prepared, placed in an appropriate container, and labeled for treatment of an indicated condition.

### EXAMPLES

Synthesis Materials and Methods for representative compounds. Various approaches using routine chemistry can be adopted to the synthesis of compounds of Formula I. The scheme below merely illustrates one example route.

Phenethylamines and phenylisopropylamines were synthesized starting from the substituted benzaldehyde using a Henry reaction to give the nitrostyrene. In other cases, these were purchased commercially. Many of the substituted benzaldehydes required were commercially available or were synthesized using standard methods familiar to those experienced in the art. The substituted nitrostyrenes were then reduced using LAH or alane (AlH₃) at room temperature (in some cases mild heat can be used to speed up rate) THF under argon atmosphere to give after an acid base workup the target primary amine. In some cases, additional substitutions were made directly on the substituted phenethylamine or phenylisopropylamine using for example electrophilic aromatic substitutions (e.g., nitration, chlorination, or bromination). Once purified (crystallization, flash chromatography or short path vacuum distillation), the phenethylamine or phenylisopropylamine underwent reductive amination to produce the final compound: this was done by pre-forming the imine from a mixture of the phenethylamine and a substituted benzaldehyde in methanol in the presence of 3A angstrom molecular sieves, equivalent aldehyde or ketone and then reduced using NaBH₄. Compounds were then purified by column chromatography and/or conversion to the HCl salt. All final compounds were prepared as HCl salts.

Alternative synthetic routes familiar to those experienced in the art include but are not limited to, coupling of the appropriately substituted phenylacetic acid with the appropriate benzylamine or comparable amine, following by hydride reduction to give the substituted N-substituted-phenethylamine. Another alternate is SN2 alkylation of the phenethylamine with benzylbromide (or any equivalent alkylhalide or other electrophilic alkylating reagent). Finally reductive amination can be performed using an appropriately substituted phenylacetaldehyde with an appropriately substituted amine (e.g., substituted benzylamine). In all cases, additional substitutions can be made on the obtained N-substituted-phenylalkylamine to provide the target compound such as but not limited by coupling reactions, nucleophilic substitutions or electrophilic aromatic substitutions.

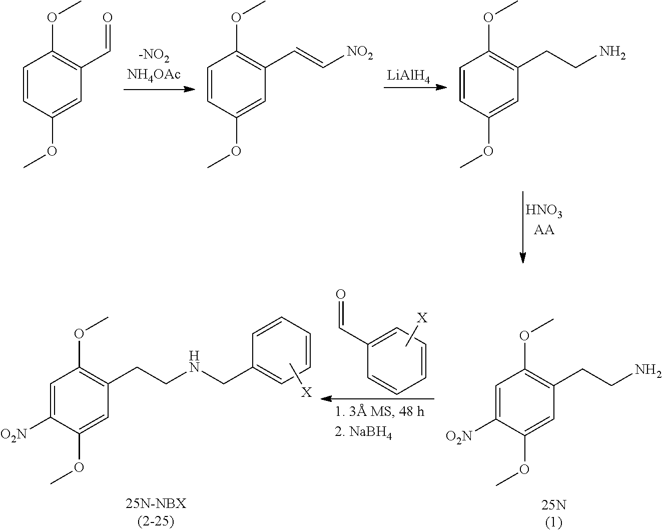

N-benzyl-compounds were synthesized by reductive amination (using NaBH₄) of the pre-formed imine obtained by treating the primary amine 2,5-dimethoxy-4-nitrophenethylamine (2C—N) with the respective aldehyde in dry methanol and THE in the presence of 3 Å molecular sieves in the dark under an argon atmosphere for at least 48 hours. 1,4-dimethoxy-2-[(1E)-2-nitroethenyl]benzene

0.0722 mol (12.0 g) 2,5-dimethoxybenzaldehyde was dissolved in 25 mL nitromethane containing 0.0157 mol (1.21 g) ammonium acetate. The reaction was heated at 80 C on a water bath for 6 hours. After which the solution rapidly set to a solid orange cake. This was allowed to sit at room temperature overnight, the solids were then collected by gravity filtration, dissolved in 50 mL boiling isopropanol and allowed to sit at room temperature for several days. The deep orange crystals were collected by vacuum filtration and dried to give 0.0529 mol (11.6 g) 1,4-dimethoxy-2-[(1E)-2-nitroethenyl]benzene as orange crystalline needles. A second crop containing an additional 0.4 g was obtained after recrystallization from 10 mL 200 proof ethanol. Total yield=12.0 g (79.5%).

### 2-(2,5-dimethoxyphenyl)ethan-1-amine (2C—H)

0.0554 mol (11.6 g) of 1,4-dimethoxy-2-[(1E)-2-nitroethenyl]benzene was dissolved in 150 mL anhydrous THF and added slowly dropwise over 1 hour to a stirred suspension of 0.166 mol (6.3 g) lithium aluminium hydride (LAH) in 100 mL anhydrous THF on an ice-water bath under argon. After the addition was completed, the grey solution was allowed to recover to room temperature, at which point it was heated to a mild reflux. Progress was monitored by TLC and MS-ASAP. After 3 hours the reaction was completed. The reaction was cooled on an ice-water bath and excess hydride was quenched by the slow dropwise addition of H₂O:THF (3:1) over ˜30 minutes. The solution was then diluted with 200 mL EtOAc and the inorganics removed by gravity filtration. The solids were washed heavily with EtOAc (˜200 mL). The resulting EtOAc solution was extracted with aqueous 1N HCl (3×150 mL). The pooled aqueous solutions were then made basic by addition of KOH pellets. The resulting cloudy solution was then extracted with EtOAc (3×100 mL), each extraction washed with 10 mL brine and then pooled and dried with anhydrous sodium sulfate. The solvent was removed using rotary evaporation to give an amber oil. This crude freebase was immediately distilled using a kugelrohr (170-200° C.) to give 5.2 g of 2C—H as a colorless oil (51.8% yield). This oil set to a white solid upon storage at −20 C under argon.

### 2-(2,5-dimethoxy-4-nitrophenyl)ethan-1-amine (2C—N)

2C—N was synthesized using a modification of the method described by Shulgin and Shulgin (1991). 0.027589 mol (5.0 g) 2,5-dimethoxyphenethylamine freebase was dissolved in 50 mL glacial acetic acid and placed on ice while vigorously stirring. 16.5 mL of 70% nitric acid was added dropwise over several minutes. The initially clear solution turned yellow upon addition of the nitric acid. Stirring on ice was continued and after 12 minutes a spatula was used to scratch the inside of the flask resulting in the precipitation of a small amount of yellow crystals. The solution then set to a yellow crystalline mass over 1 minute. It was stirred for an additional 20 minutes, at which point 75 mL of diethyl ether (Et₂O) was slowly added. The resulting light-yellow crystals were collected onto Whatman paper by vacuum filtration, washed with additional Et₂O and dried at room temperature to give 6.66 g of fluffy canary yellow crystals. An additional 0.64 g of material (darker sparkling yellow crystals) was collected as a slower precipitate from the combined filtrate and washes. Total yield: 7.3 g (91.43%) of 2C—N nitrate. This material was dissolved in water, basified with excess KOH pellets, extracted with EtOAc (3×75 mL), and then the pooled extracts were washed with brine, dried over anhydrous sodium sulfate and concentrated under rotary evaporation to give 2C—N freebase as a yellow-orange waxy solid that set to single solid mass in near quantitative yield from the nitrate salt.

2C—N HCl: The freebase was dissolved in 20 mL ethanol (200 proof) and titrated to an acidic pH (pH<3) with concentrated HCl while stirring. The solvent was then evaporated under warm air flow. Additional EtOH was added and evaporation repeated until all excess water and acid were gone (˜4×10 mL volumes of EtOH). This resulted in light yellow powder which was washed with Et₂O (10 mL) and dried with gentle heating. The resulting solids were recrystallized by dissolving in ˜5 mL EtOH followed by the addition of ˜20 mL Et₂O and storing at room temperature (˜1 hour) followed by −20° C. overnight. The resulting crystals were then washed with Et₂O (2×10 mL) followed by EtOAc (5 mL). This was repeated for a total of three crystallizations to give light yellow crystalline solids of 2C—N HCl that were then dried in a vacuum desiccator for ˜48 hours, mp=201.0-202.3° C. (Lit: 193-195° C.[Shulgin and Shulgin 1991]. HRMS: Observed: 227.1015 (100), Theoretical: C₁₀H₁₄N₂O₄ +H: 227.1026, Δppm: −4.843.

Benzyl[2-(2,5-dimethoxy-4-nitrophenyl)ethyl]amine (25N—NB) (2)

0.00088 mol (200 mg) 2C—N freebase and 0.001056 mol (112 mg) benzaldehyde were dissolved in 10 mL dry (3A molecular sieves) methanol and 2 mL anhydrous THF containing ˜1 g 3A molecular sieves. The reaction was sealed under argon and protected from light for 4 days. After which, the reaction was placed on an ice-water bath under argon flow, at which point 0.0044 mol (166 mg) NaBH₄ was added in small portions over 10 minutes with vigorous stirring. Next, the reaction was mixed for an additional hour at which point it was removed from the ice-water bath and left to sit for 4 hours with occasional mixing. The reaction was then quenched by slow addition of the solution to 300 mL 2N aqueous HCl. The solution was washed with EtOAc (2×60 mL). The organic washes were pooled and extracted twice with 2N aqueous HCl (3×60 mL). The acidic aqueous phases were pooled, made basic with KOH pellets until cloudy and extracted with EtOAc (3×60 mL). The organic extracts were washed with brine (10 mL), pooled, dried over anhydrous magnesium sulfate and evaporated under rotary evaporation to give a yellow oil. This crude freebase was purified via flash column chromatography on silica gel with hexanes: EtOAc (3:2) containing 1% triethylamine, slowing increasing the EtOAc to 50%. Pure fractions were identified using MS-ASAP and TLC and combined to give 140 mg (50.4% yield) of a light yellow oil. HCl Salt: Prepared as described for 2C—N to give a beige-tan crystalline powder (mp: 220.0-221.0° C.). HRMS: Observed: 317.147951 (100), Theoretical: C₁₇H₂₀N₂O₄+H: 317.14958 (100), Δppm: −0.22.

### 2-({[2-(2,5-dimethoxy-4-nitrophenyl)ethyl]amino}methyl)phenol (25N—NBOH) (3)

Prepared as described for 25N—NB (2) using 0.00088 mol (200 mg) 2C—N and 0.00132 mol (185 μL) 2-hydroxybenzaldehyde to give an amber oil (column chromatography). HCl salt prepared as described for 2C—N HCl to give 203.3 mg (62.6% yield) light yellow crystalline solids (mp: 205-206.7° C.). HRMS: Observed: 333.1443 (100), Theoretical: C₁₇H₂₀N₂O₅+H: 333.1445, Δppm: −0.600. Elemental Analysis: Calc: C, 55.36; H, 5.74: N, 7.6 Found: C, 54.92: H, 5.81: N, 7.38

### [2-(2,5-dimethoxy-4-nitrophenyl)ethyl] [(2-methoxyphenyl)methyl]amine (25N—NBOMe) (4)

Prepared as described for 25N—NB (2) using 0.0088 mol (2 g) 2C—N and 0.01056 mol (1.44 g) 2-methoxybenzaldehyde to give 1.95 g (64% yield) of a dark yellow oil (after flash column chromatography). HCl salt prepared as described for 2C—N HCl to give large sparkling transparent yellow needles (mp: 166.4-167.8° C.). HRMS: 347.1599 (100), Theoretical: C₁₈H₂₂N₂O₅+H: 347.1601, Appm: −0.5761. Elemental analysis: Calc: C, 56.47; N, 6.06: N, 7.32 Found: C, 56.17; H, 5.87; N, 7.43.

[(2,2-difluoro-2H-1,3-benzodioxol-4-yl)methyl][2-(2,5-dimethoxy-4-nitrophenyl)ethyl]amine (25N-NBMDF2) (13)

Prepared as described for 25N—NB (2) using 0.00088 mol (200 mg) 2C—N and 0.00132 mol (208 mg) 2,2-difluoro-1,3-benzodioxole-4-carboxaldehyde to give a yellow oil (after flash column chromatography) that was crystallized (e EtOAc:hexanes) to a yellow crystalline solid. HCl salt prepared as described for 2C—N HCl to give 87 mg (24.9% yield) of beige crystalline solids (mp: 208.4-209.8° C.). HRMS: Observed: 397.11980 (100), Theoretical: C₁₈H₁₈F₂N₂O₆+H, 397.12057 (100), Δppm: −1.93.

{[2-(difluoromethoxy)phenyl]methyl}[2-(2,5-dimethoxy-4-nitrophenyl)ethyl]amine (25N—NBOCF2H) (11)

Prepared as described for 25N—NB (2) using 0.00088 mol (200 mg) 2C—N and 0.00088 mol (151 mg) 2-dlfluoromethoxy-benzaldehyde to give a golden oil. HCl salt prepared as for 2C—N HCl to give 65 mg (17.6% yield) canary yellow solids (mp: 197.5-199.0° C.). HRMS: 383.1408 (100), Theoretical C₁₈H₂₀F₂N₂O₅+H: 383.1413, Δppm: −1.305. Elemental Analysis: C₁₈H₂₁ClF₂N₂O₅-0.18H₂O, Calc: C, 51.22; H, 5.10; N, 6.63, Found: C, 50.85; H, 5.18; N, 6.41.

### [2-(2,5-dimethoxy-4-nitrophenyl)ethyl]({[2-(trifluoromethyl)phenyl]methyl}) amine (25N—NBCF3) (14)

Prepared as described for 25N—NB (2) using 0.00088 mol (200 mg) 2C—N and 0.00132 mol (177 μL) 2-(trifluoromethyl)benzaldehyde to give a transparent yellow oil (after flash column chromatography). HCl salt prepared as described for 2C—N HCl to give 166 mg (44.8% yield) of fluffy yellow needles (mp: 156.7-159.1° C.). HRMS: Observed: 385.13662 (100) Theoretical: C₁₈H₁₉F₃N₂O₄+H, 385.13697 (100), Appm: 0.91.

### [2-(2,5-dimethoxy-4-nitrophenyl)ethyl][(2-nitrophenyl)methyl]amine (25N—NBNO₂) (15)

Prepared as described for 25N—NB (2) using 0.00088 mol (200 mg) 2C—N and 0.00158 mol (239 mg) 2-nitrobenzaldehyde to give a transparent golden oil which set to an opaque gold solid. The solids were purified by crystallization from EtOAc diluted with hexanes to give 195 mg (61.3% yield) of bright yellow salt-granule-like crystals. HCl salt prepared as described for 2C—N HCl to give a yellow crystalline powder (mp: 200.5-201.7° C.). HRMS: 362.13458 (100), Theoretical: C₁₇H₁₉N₃O₆+H, 362.13466 (100), Δppm: −0.22.

({[1,1′-biphenyl]-2-yl}methyl)[2-(2,5-dimethoxy-4-nitrophenyl)ethyl]amine (25N—NBPh) (17)

Prepared as described for 25N—NB using 0.00088 mol (200 mg) 2C—N and 0.00132 mol (240.5 mg) biphenyl-2-carboxylate to give a yellow oil (after flash column chromatography). HCl salt prepared as described for 2C—N HCl to give 104.3 mg (27.6% yield) of light yellow fluffy crystalline needles mp: 181.5-182.5° C.). HRMS: Observed: 393.18068 (100), Theoretical: C₂₃H₂₄N₂O₄, 393.18088 (100), Δppm: −0.51.

### [2-(2,5-dimethoxy-4-nitrophenyl)ethyl][(naphthalen-1-yl)methyl]amine (25N—N-1-Nap) (16)

Prepared as described for 25N—NB (2) using 0.00088 mol (200 mg) 2C—N and 0.00132 mol (179 μL) 1-napthaldehyde to give a yellow oil which was purified by crystallization (2X) from EtOAc and hexanes at 0 C to give 103 mg of yellow needles (32% yield). An additional 200 mg of a water soluble solid was recovered from the organic washes suspected HCl. Total % yield: 83%. The HCl salt was prepared as described for 2C—N HCl to give fluffy biege crystalline solid mp: 191.8-192.3° C.). HRMS: Observed: 367.16534 (100), Theoretical: C₂₁H₂₂N₂O₄, 367.16523 (100), Δppm: 0.29.

### [2-(2,5-dimethoxy-4-nitrophenyl)ethyl][(3-methylphenyl)methyl]amine (25N—NB-3-Me) (22)

Prepared as described for 25N—NB (2) using 0.000663 mol (150 mg) 2C—N and 0.001326 mol (156 μL) p-tolualdehyde to give a yellow-orange solid. This was crystalized (2X) from EtOAc and hexanes stored at 0° C. to give 160 mg (73.1% yield) of crystalline orange needle clusters. HCl salt prepared as described for 2C—N HCl to give a beige-yellow crystalline powder (mp: 194.4-195.5° C.). HRMS: Observed: 331.1645 (100), Theoretical: C₁₈H₂₂N₂O₄+H, 331.1652 (100), Δppm: −2.11.

### [2-(2,5-dimethoxy-4-nitrophenyl)ethyl] [(4-methylphenyl)methyl]amine (25N—NB-4-Me) (23)

Prepared as described for 25N—NB (2) using 0.000663 mol (150 mg) 2C—N and 0.001326 mol (156 μL) m-tolualdehyde to give 115 mg (52.5% yield) of a yellow oil (after flash column chromatography). HCl salt prepared as described for 2C—N HCl to give fluffy slightly-yellow crystalline powder (mp: 182.0-184.0° C.). HRMS: Observed: 331.1645 (100), Theoretical: C₁₈H₂₂N₂O₄+H, 331.1652 (100), Δppm: −2.11.

### [2-(2,5-dimethoxy-4-nitrophenyl)ethyl][(3-fluorophenyl)methyl]amine (25N—NB-3-F) (24)

Prepared as described for 25N—NB (2) using 0.000663 mol (150 mg) 2C—N and 0.001326 mol (140.7 μL) 3-fluorobenzaldehyde to give 115.4 mg (52% yield) of a yellow oil (after flash column chromatography). HCl salt prepared as described for 2C—N HCl to give a light-yellow crystalline needles (mp: 216.2-217.5° C.). HRMS: Observed: 335.1393 (100), Theoretical: C₁₇H₁₉FN₂O₄+H, 335.1402 (100), Δppm: −2.68.

### [2-(2,5-dimethoxy-4-nitrophenyl)ethyl][(4-fluorophenyl)methyl]amine (25N—NB-4-F) (25)

Prepared as described for 25N—NB (2) using 0.000663 mol (150 mg) 2C—N and 0.001326 mol (156 μL) 4-fluorobenzaldehyde to give small circular orange crystalline clusters. HCl salt prepared as described for 2C—N HCl to give 106 mg (43% yield) of orange-brown fluffy crystalline needles (mp: 180.0-181.0° C.). HRMS: Observed: 335.1396 (100), Theoretical: C₁₇H19FN₂O₄+H, 335.1402 (100), Δppm: −1.79.

### 3-({[2-(2,5-dimethoxy-4-nitrophenyl)ethyl]amino}methyl)phenol (25N—NB-3-OH) (21)

Prepared as described for 25N—NB (2) using 0.00088 mol (200 mg) 2C—N and 0.001056 mol (129 mg) 3-hydroxy benzaldehyde to give a yellow oil which solidified to a yellow solid upon sitting. This was purified by crystallization (dissolve in boiling 10 mL EtOAc, 2 mL ethanol followed by dilution with 20 mL hexanes and storing at 0 C) to give 195 mg transparent tan needles (66.8% yield). HCl salt prepared as described for 2C—N HCl to give transparent orange flat edged rectangular crystals (mp: 194.0-195.5° C.). HRMS: Observed: 333.14434 (100), Theoretical: C₁₇H₂₀N₂O₅—H+, 333.14434 (100), Δppm: −0.48.

### 2-({[2-(2,5-dimethoxy-4-nitrophenyl)ethyl]amino}methyl)-6-methylphenol (25N—NB-2-OH-3-Me) (18)

Prepared as described for 25N—NB (2) using 0.00088 mol (200 mg) 2C—N and 0.00132 mol (179.7 mg) 2-hydroxy-3-methylbenzaldehyde to give a yellow oil. HCl salt prepared as for 2C—N HCl to give 180 mg (53.4% yield) transparent neon yellow crystalline solids (mp: 177.6-179.5° C.). HRMS: Observed, 347.1588 Theoretical: C₁₈H₂₂N₂O₅+H, 347.1601, Δppm: −3.744.

### [2-(2,5-dimethoxy-4-nitrophenyl)ethyl][(3-fluoro-2-methoxyphenyl)methyl]amine (25N—NB-2-MeO-3-F) (19)

Prepared as described for 25N—NB (2) using 0.00088 mol (200 mg) 2C—N and 0.00088 mol (136 mg) 2-methoxy-3-dluorobenzaldehyde to give a golden oil. HCl salt prepared as for 2C—N HCl to give 130 mg (36.9% yield) transparent yellow needles (mp: 155.0-156.7° C.). HRMS: Observed, 365.1500 Theoretical: C₁₈H₂₁FN₂O₅+H, 365.1507,Δppm: −1.91.

### 2-(6-methoxy-2H-1,3-benzodioxol-5-yl)ethan-1-amine (2C-2)

0.0278 mol (5.0 g) 6-methoxy-2H-1,3-benzodioxole-5-carbaldehyde was dissolved in 50 mL nitromethane and 0.5 g ammonium acetate was added followed by 8 drops of cyclohexylamine. The solution was sealed under argon and heated for 5 hours in an 80° C. water bath and left to sit at room temperature overnight. The next day dark orange crystals had precipitated and the reaction was placed at −20° C. for 24 hours. The crystalline solids were collected by decanting and washing sparingly with ethanol, followed by drying under argon flow: The resulting solids were boiled in 100 mL methanol with grinding, and were only partially soluble. The suspension was placed in the freezer for an additional 24 hours, at which point the solids were collected by gravity filtration, washed twice with 10 mL ethanol and dried to give 4.3 g orange-red (69.5%).

0.01927 mol (4.3 g) 5-methoxy-6-[(1E)-2-nitroethenyl]-2H-1,3-benzodioxole were dissolved in 60 mL anhydrous THF and added dropwise over 30 minutes to a stirred suspension of 0.0578 mol (2.19 g) LAH in 100 mL anhydrous THF which was kept under argon and on an ice-water bath. After the addition the reaction was allowed to recover to room temperature and then placed under reflux. Reflux was maintained for ˜3 hours at which point TLC showed complete conversion. The workup was performed as described for 2C—H to give an amber oil of the crude product after evaporation of the EtOAc solvent. The crude freebase 2C-2 was purified via flash column chromatography (silica gel) starting with EtOAc:hexanes (4:1) containing 0.4% triethylamine and increasing to 10% ethanol in EtOAc (0.4% triethylamine). Desired fractions were pooled using MS (ASAP) and TLC to give 2.59 g of a beige waxy solid (68.9% yield). 2C-2 HCl salt prepared as described for 2C—N HCl (with two additional crystallizations, 5-total, from MeOH:Et₂O) to give 2C-2 HCl as off-white powder (mp: 227.8-229.2° C.). [2-(6-methoxy-2H-1,3-benzodioxol-5-yl)ethyl][(naphthalen-1-yl)methyl]amine (39)

Prepared as described for 25N—NB using 0.000866 mol (200 mg) 2C-2 HCl and 0.00184 mol (287 mg) 1-napthaldehyde to give 350 mg of an amber oil (which contained material from reduced 1-napthaldehyde). HCl salt was prepared as described for 2C—N HCl to give 270.6 mg (84.0% yield) of white crystalline powder (mp: 190.8-191.1° C.). HRMS: Observed: 336.1582 (100), Theoretical: C₂₁H₂₁NO₃+H: 336.1594 (100), Δppm. 3.57.

### 4-bromo-3,5-dimethoxyphenethylamine (35B)

A dry, two-neck round-bottom flask with a stirbar was charged with anhydrous THF (150 mL) and the flask was cooled to 0° C. With vigorous stirring, lithium aluminum hydride (2.7 g, 71.1 mmol) was added in one portion and the flask was flushed with argon. Fuming sulfuric acid (1.6 mL, 3.08 g, 31.4 mmol) was added dropwise at 0° C. over 5 min via syringe and then the flask was fitted with a dry addition funnel. A solution of 4-bromo-3,5-dimethoxynitrostyrene (6.0 g, 20.8 mmol) in dry THF (100 mL) was added to the addition funnel and the solution was added dropwise over 1 hr at 0° C. The reaction was then stirred for 3 hr at room temperature and then quenched by dropwise addition of cold 1:1 THF: dH₂O (˜50 mL) at 0° C. The reaction was basified with the addition of KOH solution and the resulting suspension was gravity filtered. The filter cake was washed with EtOAc (3×50 mL) and the filtrate was transferred to a separatory funnel. The aqueous layer was extracted with EtOAc (3×100 mL) and the combined organics were washed with brine, dried over Na₂SO₄, and concentrated to afford an oily substance. The crude product was dissolved in EtOH (˜30 mL) and a stoichiometric equivalent of c. HCl was added. Solvent and excess HCl were removed with a stream of warm air and the crude salt was washed with Et₂O (3× ˜10 mL). The salt was crystalized by dissolving in a minimum volume of hot EtOH and layering with Et₂O. Several crops of solid were isolated to provide 4-bromo-3,5-dimethoxyphenethylamine hydrochloride (1.67 g. 27.1%) as light-yellow solids, (mp: 231.3-232.1ºC). HRMS: Observed: 260.0276 (100), Theoretical: C₁₀H₁₄BrNO₂+H: 260.0281 (100), Δppm: 1.92.

### 2-(4-bromo-3,5-dimethoxyphenyl)-N-[(naphthalen-1-yl)-methyl]ethan-1-amine (31)

0.000674 mol (200 mg) 35B HCl salt and 0.00101 mol (137 μl) 1-naphthaldehyde were dissolved in 15 mL dry (3 Å molecular sieves) methanol and 2 mL anhydrous THF containing ˜1.5 g 3 Å molecular sieves. 0.001348 mol (187 μl) Triethylamine (TEA) was added to the reaction to convert the salt to its freebase. The reaction was sealed under argon and protected from light for greater than 48 hours. A time duration greater than 48 hours did not leave any significant changes in the reaction as observed. After which, the reaction was placed on an ice-water bath and argon flow, at which point (˜2.5M equivalent, 0.00177 mol, 67 mg) NaBH₄ was added in small portions over 10 minutes with vigorous mixing. Ice-bath was removed, and the reaction was continued for an additional two hours. Base extraction was then carried out for the reaction where KOH pellets were added (until cloudy) and extracted with EtOAc (3×60 mL). Organic extracts were washed with brine (10 mL), pooled, dried over anhydrous sodium sulfate and evaporated under rotary evaporation to give a yellow oil. This crude freebase was purified via flash column chromatography on silica gel with hexane: EtOAc (1:4) containing 0.5% TEA. The EtOAc was slowly increased to 100%. Pure fractions were identified using MS-ASAP and TLC and combined to give 250 mg (92.6% yield) of yellow oil. HCl Salt: Prepared as described for 2C—N to give a white, crystalline powder (mp: 232.8-234.3° C.). HRMS: Observed: 400.0902 (100), Theoretical: C₂₁H₂₂BrNO₂+H: 400.0907 (100), Δppm: 1.25.

### 2-(((2-bromo-4,5-dimethoxyphenethyl)-amino)-methyl)-6-methylphenol (43)

Prepared as described for (31) using 0.00154 mol (400 mg) iso-2CB as free base and 0.00231 mol (280.06 μL) 3-methylsalicylaldehyde to give 590 mg HCl salt (91.90% yield). No TEA was added during the reaction as the iso-2CB was a free base and the procedure was same as that of (31) except that NH₄OH was used for base extraction. HCl salt was prepared as described for 2C—N HCl to give white, shiny particles (mp: 169.4-169.9° C.). HRMS: Observed: 381.0876, Theoretical: C₁₈H₂₂BrNO₃+H: 381.0889, Δppm: 3.41. 1H NMR (400 MHZ, DMSO) δ 9.07 (s, 2H)*, 8.90 (s, 1H)*, 7.29-7.21 (m, 1H), 7.16 (d, J=7.4 Hz, 1H), 7.13 (s, 1H), 7.00-6.96 (m, 1H), 6.82 (t, J=7.4 Hz, 1H), 4.17 (s, 2H), 3.77 (s, 3H)**, 3.75 (s, 3H)**, 3.14-3.07 (m, 2H), 3.07-2.99 (s, 2H), 2.22 (s, 3H). *, **, *** coalescing. ¹³C NMR (101 MHZ, DMSO) δ 153.78 (s, 1C), 148.52 (s, 1C), 148.41 (s, 1C), 131.77 (s, 1C), 129.33 (s, 1C), 128.18 (s, 1C), 125.67 (s, 1C), 119.69 (s, 1C), 119.33 (s, 1C), 115.64 (s, 1C), 114.02 (s, 1C), 113.31 (s, 1C), 55.91 (s, 1C), 55.73 (s, 1C), 46.11 (s, 1C), 45.52 (s, 1C), 31.34 (s, 1C), 16.68 (s, 1C).

### N-(3-iodobenzyl)-2-(2,4,5-trimethoxyphenyl)-ethan-1-amine (53)

Prepared as described for (31) using 0.00121 mol (300 mg) 250 HCl and 0.00147 mol (340 mg) 3-iodobenzaldehyde to give 440 mg HCl salt (78.4% yield). The procedure was same as that of 35B—N1-Nap except that ˜4 ml THF was used instead of 2 ml, and column chromatography was not carried out for purification. HCl salt was made as described for 2C—N HCl to give white, fluffy particles (mp: 155.5-156.1° C.). HRMS: Observed: 428.0703, Theoretical: C₁₈H₂₂INO₃+H: 428.0717, Δppm: 3.27. ¹H NMR (400 MHZ, DMSO) δ 9.37 (s, 2H), 7.97 (s, 1H), 7.78 (d, J=7.9 Hz, 1H), 7.59 (d. J=7.7 Hz, 1H), 7.24 (t. J=7.8 Hz, 1H), 6.80 (s, 1H), 6.68 (s, 1H), 4.11 (s, 2H), 3.77 (s, 3H)*, 3.76 (s, 3H)*, 3.69 (s, 3H), 3.06-2.96 (m, 2H), 2.92-2.84 (m, 2H). ¹³C NMR (101 MHZ, DMSO) δ 151.43 (s, 1C), 148.64 (s, 1C), 142.53 (s, 1C), 138.55 (s, 1C), 137.52 (s, 1C), 134.59 (s, 1C), 130.68 (s, 1C), 129.55 (s, 1C), 115.77 (s, 1C), 115.07 (s, 1C), 98.46 (s, 1C), 94.90 (s, 1C), 56.40 (s, 1C), 56.17 (s, 1C), 55.89 (s, 1C), 48.91 (s, 1C), 46.33 (s, 1C), 25.97 (s, 1C).

### N-(2-(thiophen-2-yl) benzyl)-2-(2,4,5-trimethoxyphenyl)-ethan-1-amine, (59)

Prepared as described for (31) using 0.00087 mol (215 mg) 250 HCl and 0.00130 mol (245 mg) 2-(thiophen-2-yl)-benzaldehyde to give 260 mg HCl salt (80.2% yield). The procedure was same as that of (31) except that ˜4 ml THF was used instead of 2 ml, and column chromatography was not carried out for purification. HCl salt was made as described for 2C—N HCl to give white, flaky particles (mp: 158.4-159.8° C.). HRMS: Observed: 384.1613, Theoretical: C₂₀H₂₅O₃SN+H: 384.1628, Δppm: 3.90. ¹H NMR (400 MHZ, DMSO) δ 9.56 (s, 2H), 7.88 (d, J=7.1 Hz, 1H), 7.69 (dd, J=5.1, 1.1 Hz, 1H), 7.54-7.49 (m, 1H)*, 7.49-7.43 (m, 2H)*, 7.25 (dd, J=3.5, 1.0 Hz, 1H), 7.19 (dd, J 5.1, 3.5 Hz, 1H), 6.76 (s, 1H), 6.66 (s, 1H), 4.24 (s, 2H), 3.76 (s, 3H), 3.73 (s, 3H), 3.68 (s, 3H), 3.07-2.92 (m, 2H), 2.91-2.76 (m, 2H). * =coalescing. 13C NMR (101 MHZ, DMSO) δ 151.38 (s, 1C), 148.60 (s, 1C), 142.50 (s, 1C), 140.05 (s, 1C), 134.27 (s, 1C), 131.03 (s, 1C), 130.38 (s, 1C), 129.89 (s, 1C), 128.83 (s, 1C), 128.44 (s, 1C), 128.03 (s, 1C), 127.88 (s, 1C), 127.22 (s, 1C), 115.74 (s, 1C), 114.95 (s, 1C), 98.41 (s, 1C), 56.41 (s, 1C), 56.14 (s, 1C), 55.88 (s, 1C), 47.11 (s, 1C), 46.62 (s, 1C), 25.81 (s, 1C).

### 1-(naphthalen-1-yl)-N-(2,4,5-trimethoxyphenethyl)-ethan-1-amine (66)

Prepared as described for (31) using 0.00121 mol (300 mg) 250 HCl and 0.00182 mol (276 μl) 1-acetonaphthone to give a crude yellow oil. This crude freebase was purified via flash column chromatography on silica gel with hexane: EtOAc (3:2) containing 0.5% TEA. The EtOAc was slowly increased to 100%. Pure fractions were identified using MS-ASAP and TLC and combined to give 150 mg (33.9% yield) of yellow oil. HCl salt was made as described for 2C—N HCl to give white, shiny particles (mp: 195.2-196.4° C.). HRMS: Observed: 366.2051, Theoretical: C₂₃H₂₇NO₃+H: 366.2064, Δppm: 3.55. ¹H NMR (400 MHZ, DMSO) δ 10.05 (s, 1H), 9.36 (s, 1H), 8.22 (d, J=8.3 Hz, 1H), 8.05-7.95 (m, 3H), 7.67-7.54 (m, 3H), 6.71 (s, 1H), 6.60 (s, 1H), 5.32 (dd, J=11.8, 6.0 Hz, 1H), 3.73 (s, 3H), 3.64 (s, 3H), 3.60 (s, 3H), 3.10-2.97 (m, 1H)*, 2.97-2.82 (m, 3H)*, 1.70 (d, J=6.7 Hz, 3H). * coalescing. ¹³C NMR (101 MHZ, DMSO) δ 151.31 (s, 1C), 148.59 (s, 1C), 142.48 (s, 1C), 134.15 (s, 1C), 133.33 (s, 1C), 130.28 (s, 1C), 128.91 (s, 2C), 126.90 (s, 1C), 126.16 (s, 1C), 125.53 (s, 1C), 124.27 (s, 1C), 122.61 (s, 1C), 115.79 (s, 1C), 114.98 (s, 1C), 98.44 (s, 1C), 56.37 (s, 1C), 55.96 (s, 1C), 55.84 (s, 1C), 51.85 (s, 1C), 45.02 (s, 1C), 26.21 (s, 1C), 19.83 (s, 1C).

### N-(benzo[b]thiophen-7-ylmethyl)-2-(2,4,5-trimethoxyphenyl)-ethan-1-amine, (73)

Prepared as described for (31) using 0.00125 mol (310 mg) 250 HCl and 0.00191 mol (310 mg) benzo(b) thiophene-7-carbaldehyde to give 420 mg HCl salt (85.2% yield). The procedure was same as that of (31) except that NH₄OH was used for base extraction. HCl salt was made as described for 2C—N HCl to give white, shiny particles (mp: 191.4.-192.5° C.). HRMS: Observed: 358.1473, Theoretical: C₂₀H₂₃NO₃S+H: 358.1471, Δppm: 0.56. ¹H NMR (400 MHZ, DMSO) δ 9.53 (bs, 2H), 7.96 (d, J=7.9 Hz, 1H), 7.87 (d. J=5.4 Hz, 1H), 7.74-7.7.65 (m, 1H), 7.57 (d. J=5.4 Hz, 1H), 7.50 (t, J=7.6 Hz, 1H), 6.82 (s, 1H), 6.68 (s, 1H), 4.42 (s, 2H), 3.77 (s, 3H)*, 3.75 (s, 3H)*, 3.70 (s, 3H), 3.22-3.11 (m, 2H), 2.99-2.89 (m, 2H). * coalescing. 13C NMR (101 MHZ, DMSO) δ 151.42 (s, 1C), 148.63 (s, 1C), 142.54 (s, 1C), 140.12 (s, 1C), 139.42 (s, 1C), 127.41 (s, 1C), 126.41 (s, 1C), 125.12 (s, 1C), 124.71 (s, 2C), 124.28 (s, 1C), 115.75 (s, 1C), 115.05 (s, 1C), 98.50 (s, 1C), 56.38 (s, 1C), 56.13 (s, 1C), 55.88 (s, 1C), 48.55 (s, 1C), 46.93 (s, 1C), 25.94 (s, 1C).

### N-(dibenzo-[b, d]-furan-4-ylmethyl)-2-(2,4,5-trimethoxyphenyl)-ethan-1-amine (74)

Prepared as described for (31) using 0.0012 mol (300 mg) 250 HCl and 0.00182 mol (357 mg) dibenzo-[b, d]-furan-4-carbaldehyde to give white solids. HCl salt was prepared as described for 2C—N HCl to give 240 mg (46.3% yield) of fluffy, white crystalline powder. HRMS: Observed: 392.1850, Theoretical: C₂₄H₂₅NO₄+H: 392.1856, Δppm: 1.53. ¹H NMR (400 MHZ, DMSO) δ 9.55 (s, 2H), 8.22 (dd, J=7.5, 0.9 Hz, 1H)*, 8.20 (dd, J=7.4, 0.9 Hz, 1H)*, 7.80 (d, J=7.4 Hz, 1H)**, 7.76 (d, J=8.2 Hz, 1H)**, 7.59 (atd, J=12.0, 1.2 Hz, 1H), 7.49 (t, J=7.5 Hz, 1H)***, 7.45 (td, J=7.3, 0.7 Hz, 1H)***, 6.81 (s, 1H), 6.67 (s, 1H), 4.54 (s, 2H), 3.76 (s, 3H), 3.73 (s, 3H), 3.67 (s, 3H), 3.21-3.12 (m, 2H), 2.98-2.90 (m, 2H). *, ** coalescing. ¹³C NMR (101 MHz, DMSO) δ 155.32 (s, 1C), 153.88 (s, 1C), 151.42 (s, 1C), 148.62 (s, 1C), 142.53 (s, 1C), 129.07 (s, 1C), 128.03 (s, 1C), 123.83 (s, 1C), 123.48 (s, 1C), 123.46 (s, 1C), 123.28 (s, 1C), 122.03 (s, 1C), 121.49 (s, 1C), 115.99 (s, 1C), 115.72 (s, 1C), 115.05 (s, 1C), 111.78 (s, 1C), 98.46 (s, 1C), 56.33 (s, 1C), 56.11 (s, 1C), 55.87 (s, 1C), 46.63 (s, 1C), 43.79 (s, 1C), 25.93 (s, 1C).

### N-((5-phenylthiophen-2-yl) methyl)-2-(2,4,5-trimethoxyphenyl)-ethan-1-amine, (76)

Prepared as described for (31) using 0.0012 mol (300 mg) 250 HCl and 0.00182 mol (342.6 mg) 5-phenyl-2-thiophenecarboxyladelhyde to give a transparent, amber-colored oil. HCl salt prepared as described for 2C—N HCl to give 500 mg (93.0% yield) as white crystalline needles. HRMS: Observed: 384.1629, Theoretical: C₂₂H₂₅NO₃S+H: 384.1628, Δppm: 0.26. ¹H NMR (400 MHz, DMSO) δ 9.41 (s, 2H), 7.65 (d, J=7.4 Hz, 2H), 7.48 (d, J=3.7 Hz, 1H), 7.44 (at, J=7.6 Hz, 2H), 7.38-7.31 (m, 2H), 6.81 (s, 1H), 6.68 (s, 1H), 4.38 (s, 2H), 3.77 (s, 3H)*, 3.76 (s, 3H), 3.69 (s, 3H), 3.11-3.00 (m, 2H), 2.96-2.84 (m, 2H). * coalescing. ¹³C NMR (101 MHZ, DMSO) δ 151.44 (s, 1C), 148.65 (s, 1C), 145.15 (s, 1C), 142.53 (s, 1C), 133.24 (s, 1C), 132.30 (s, 1C), 132.05 (s, 1C), 129.23 (s, 2C), 128.06 (s, 1C), 125.36 (s, 2C), 123.68 (s, 1C), 115.76 (s, 1C), 115.12 (s, 1C), 98.48 (s, 1C), 56.38 (s, 1C), 56.14 (s, 1C), 55.88 (s, 1C), 45.85 (s, 1C), 44.03 (s, 1C), 26.01 (s, 1C).

### 2-(((2-bromo-4,5-dimethoxyphenethyl) amino)-methyl)-6-methylphenol (43)

Prepared as described for (31) using 0.00154 mol (400 mg) 2-bromo-3,4-dimethoxyphenethylamine as free base and 0.00231 mol (280.06 μL) 3-methylsalicylaldehyde to give 590 mg HCl salt (91.90% yield). No TEA was added during the reaction as the iso-2CB was a free base and the procedure was same as that of (31) except that NH₄OH was used for base extraction. HCl salt was prepared as described for 2C—N HCl to give white, shiny particles (mp: 169.4-169.9° C.). HRMS: Observed: 380.0844, Theoretical: C₁₈H₂₂BrNO₃+H: 380.0856, Δppm: 3.15.

### Synthesis of 1-(4-bromo-2,6-dimethoxyphenyl)propan-2-amine (psi-DOB)

To a dry, argon flushed round-bottom flask was added anhydrous NH₄OAc (503 mg, 6.52 mmol) and nitroethane (20 mL). Cyclohexyl amine (100 μL, 86.7 mg. 0.87 mmol) and 4-bromo-2,6-dimethoxy benzaldehyde (5.0 g, 20.4 mmol) was added to the flask and the reaction was sealed with parafilm and wrapped in aluminum foil. The reaction was allowed to sit at ambient temperature for ten days. A yellow precipitate formed during the reaction and the RBF was allowed to stand at −20° C. overnight to afford yellow needle-like crystals (3.39 g). The solid was filtered and washed with 95% EtOH (2×5 mL). The filtrates were returned to −20° C. overnight to afford β-(4-bromo-2,6-dimethoxy)-α-methyl-nitrostyrene, as a yellow a solid. Solvent was decanted and the solid dried to afford a second crop of yellow solid (2.71 g, total mass was 6.1 g, ˜quantitative yield). The product was brought to the next step without further purification.

A dry, three-neck round-bottom flask with a stir bar was charged with anhydrous THF (75 mL) and the flask was cooled to (C. With vigorous stirring, lithium aluminum hydride (1.50 g, 39.5 mmol) was added in one portion and the flask was flushed with argon. The flask was fitted with a dry addition funnel and a solution of AlCl₃ (1.76 g, 13.1 mmol) in dry THF (75 mL) was added dropwise at 0° C., over 15 min. When complete, a solution of β-(4-bromo-2,6-dimethoxy)-α-methyl nitrostyrene (6.0 g, 19.8 mmol) in dry THF (100 mL) was added to the addition funnel and the solution was added dropwise over 80 min at (C. The reaction was then stirred for 2.5 hr at rt and then quenched by dropwise addition of cold 1:1 THF: H₂O (˜50 mL) at 0° C. The reaction was diluted into H₂O (˜500 mL) and basified with the addition of KOH. The resulting suspension was transferred to a separatory funnel and EtOAc (200 mL) was added. The mixture was extracted and then the aqueous layer was extracted further with EtOAc (2×100 mL). The combined organics were washed with brine, dried over Na₂SO₄, and concentrated to afford a white solid (5.45 g, quantitative). The amine was converted to the hydrochloride salt by dissolving in absolute EtOH and adding a stoichiometric equivalent of conc. HCl. Solvent and excess HCl were removed with a stream of warm air and the salt was washed with Et₂O (3×˜10 mL). The salt was crystallized three times from EtOH: Et₂O as described previously.

### 1-(4-bromo-2,6-dimethoxyphenyl)-N-(naphthalen-1-yl)-methyl)-propan-2-amine (33)

Prepared as described for (31) using 0.0013 mol (400 mg) psi-DOB HCl salt and 0.0019 mol (262.6 μL) 1-naphthaldehyde to give 490 mg HCl salt (84.2% yield). HCl salt was made as described for 2C—N HCl to give white, crystalline particles (mp: 207.9-209.2° C.). HRMS: Observed: 416.1036, Theoretical: C₂₂H₂₄BrNO₂+H:416.1043, Δppm: 1.68.

Intermediates and reagents for synthesis were obtained from Sigma-Aldrich (St Louis, MO, USA), AKSci, Alfa Aesar etc. In general, reagents were 95% pure or greater. 200 proof ethyl alcohol was obtained from Pharmaco (Greenfield Global, CT, USA). Purification via silica gel flash column chromatography was performed using Merck silica gel grade 9385 (230-400 mesh, 60 Å). A Digimelt A160 SRS digital melting point apparatus (Stanford Research Systems, Sunnyvale, CA, USA) was used for melting point using a ramp rate of 2° C./min.

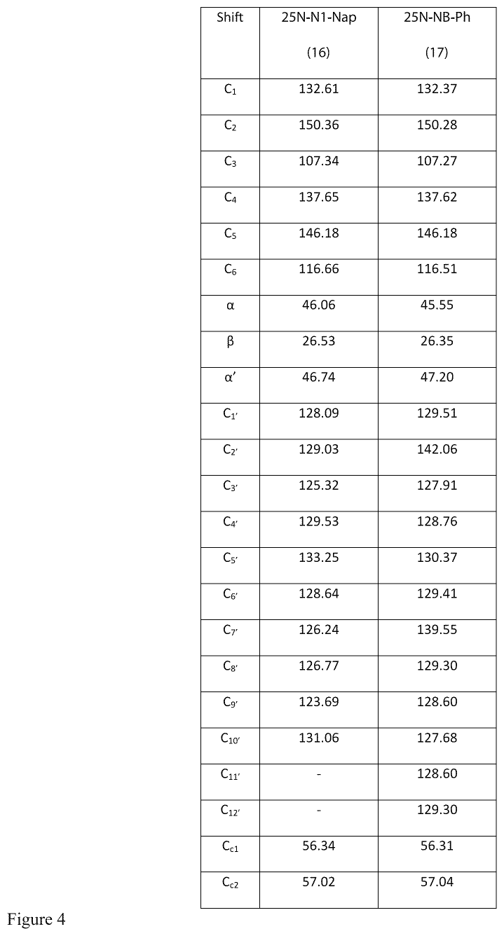

Analytical Characterizations. Once purified compounds were checked by ¹H, ¹³C, and 2-D NMR (¹H-1H COSY, ¹H-¹³C HSQC or HMQC and ¹H-¹³C HMBC to determine identity and estimate purity. Where appropriate ¹⁹F NMR was also performed. High resolution mass spectrometry is performed (<5.0 Δppm). Purity is determined using ¹H NMR and high performance liquid chromatography. Single crystal x-ray diffraction was also performed on select compounds (FIG. 4). Melting points were determined on a digimelt.

High Resolution Mass Spectrometry (HRMS). A Thermo Orbitrap Exactive Mass Spectrometer with an Orbitrap mass analyzer was utilized to calculate molecular mass. Pierce™ LTQ ESI Positive Ion Calibration Solution (ThermoFisher Scientific) was used for calibration in electrospray ionization mode. Samples were analyzed using Atmospheric Solids Analysis Probe (ASAP) technique. Data analysis was performed using the Thermo Xcalibur Qual Browser software. Identity was confirmed if Δppm was <5.0 ppm error. Setting parameters were: Aux gas flow rate-8, Spray Voltage-3.50 kV, Capillary temperature−275° C., Capillary Voltage-25.00 V, Tube Lens Voltage-65.00 V, Skimmer Voltage-14.00 V, Heater Temperature −100° C.

Elemental Analysis. C, H, N elemental analysis was determined on select compounds by Galbraith Laboratories, Inc. (Knoxville, TN).

Nuclear Magnetic Resonance. ¹H (400 MHZ) and ¹³C NMR spectra (101 MHZ) were obtained on hydrochloride salts in a solution of do-DMSO (˜20 mg/mL) (>99.9% D, Sigma-Aldrich). Measurements were made using a Bruker Avance III with PA BBO 400S1 BBF—H-D-05 Z plus probe (Bruker Corporation, Billerica, MA, USA). Internal chemical sift references were solvent (δ=2.50 and 39.52 ppm for ¹H and ¹³C spectra respectively). ¹⁹F (376.5 MHz) NMR was run as described above following addition of ˜100 μL of trichlorofluoromethane (99%+, Sigma-Aldrich) as internal reference (δ=0.0 ppm). NMR chemical shift assignments were made using chemical shift position, splitting patterns, ¹³C and ¹³C PENDANT or APT and hetero- and homo-2-D experiments including HMQC or HSQC, HMBC and COSY (45° pulse tilt).

### High Performance Liquid Chromatography (HPLC)

HPLC analyses were performed on an Agilent 1260 Infinity system. The system includes a 1260 quaternary pump VL, a 1260 ALS autosampler, a 1260 Thermostatted Column Compartment, and a DAD Multiple Wavelength Detector (Agilent Technologies, Santa Clara, CA, USA). The detection wavelengths were set at 220, 230, 254, and 280 nm. A Zorbax Eclipse XDB-C18 analytical column (5 μm, 4.6×150 mm) from Agilent Technologies was used to achieve seperation. Mobile phase A consisted of 10 mM aqueous ammonium formate buffer, which was titrated to pH 4.5. Mobile phase B consisted of acetonitrile. A 10 μL injection volume was used, The flow rate was 1.0 mL/min. The column temperature was set at 25° C. Samples were prepared at 1 mg/mL solution in 1:1 A:B. Samples were injected in duplicate with a wash of the injector (mobile phase) between each run. Each run was 10 minutes with a mobile phase ratio (isocratic) of 1:1 for A:B. Agilent ChemStation Software (Agilent Technologies) was used to analyze results.

### X-ray Diffraction Single Crystal Data and Experimental

Experiments were conducted by the Center Crystallographic For Research (Michigan State University). Single yellow needle crystals of 25N—NBPh (17) were used as provided. A suitable crystal was selected with dimensions 0.17×0.06 ×0.03 mm3. This was mounted onto a nylon loop using paratone oil on a XtaLAB Synergy, Dualflex, HyPix diffractometer. A steady T=100.00(10) K was used during data collection. ShelXT (Sheldrick, 2015) solution program using dual methods and by using Olex2 1.3-alpha (Dolomanov et al., 2009) as the graphical interface was used to solve the structure and ShelXL 2018/3 (Sheldrick, 2015) using full matrix least squares minimisation on F2 was used to refine the model.

### Design and Synthesis of Example Compounds

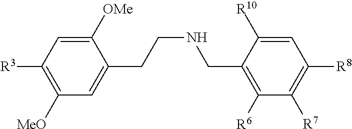

TABLE 1
Example compounds

| Compound | R⁶ | R⁷ | R⁸ | R¹⁰ | R³ |
|---|---|---|---|---|---|
| 25N-NB (2) | H | H | H | H | NO₂ |
| 25N-NBOH (3) | OH | H | H | H | NO₂ |
| 25N-NBOMe (4) | OCH₃ | H | H | H | NO₂ |
| 25N-NBOEt (5) | OCH₂CH₃ | H | H | H | NO₂ |
| 25N-NBMe (6) | CH₃ | H | H | H | NO₂ |
| 25N-NBF (7) | F | H | H | H | NO₂ |
| 25N-NBCl (8) | Cl | H | H | H | NO₂ |
| 25N-NBBr (9) | Br | H | H | H | NO₂ |
| 25N-NBI (10) | I | H | H | H | NO₂ |
| 25N-NBOCF₂H (11) | OCF₂H | H | H | H | NO₂ |
| 25N-NBOCF₃ (12) | OCF₃ | H | H | H | NO₂ |
| 25N-NBMDF₂ (13) | OCF₂O | | H | H | NO₂ |
| 25N-NBCF₃ (14) | CF₃ | H | H | H | NO₂ |
| 25N-NBNO₂ (15) | NO₂ | H | H | H | NO₂ |
| 25N-N-1-Nap (16) | (CH)₄ | | H | H | NO₂ |
| 25N-NBPh (17) | Ph | H | H | H | NO₂ |
| 25N-NB-2-OH-3-Me | OH | CH₃ | H | H | NO₂ |
| (18) | | | | | |
| 25N-NB-2-MeO-3-F | OCH₃ | F | H | H | NO₂ |
| (19) | | | | | |
| 25N-NB-2,5-DiMeO | OCH₃ | H | H | OCH₃ | NO₂ |
| (20) | | | | | |
| 25N-NB-3-OH (21) | H | OH | H | H | NO₂ |
| 25N-NB-3-Me (22) | H | CH₃ | H | H | NO₂ |
| 25N-NB-4-Me (23) | H | H | CH₃ | H | NO₂ |
| 25N-NB-3-F (24) | H | F | H | H | NO₂ |
| 25N-NB-4-F (25) | H | H | F | H | NO₂ |
| 25D-NBOMe (26) | OCH₃ | H | H | H | CH₃ |
| 25D-N1-Nap (27) | (CH)₄ | | H | H | CH₃ |
| 25D-NBPh (28) | Ph | H | H | H | CH₃ |

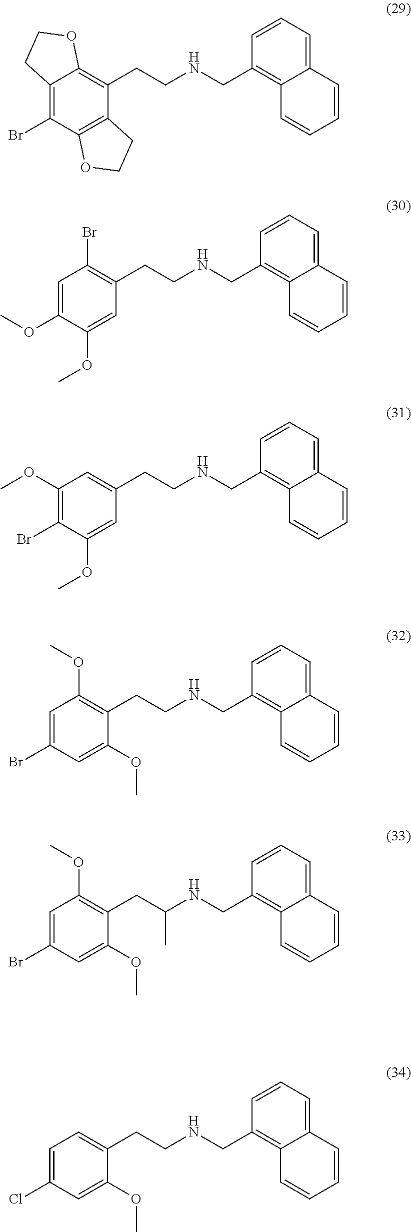 [SMILES: Brc1c2c(c(CCNCc3cccc4ccccc34)c3c1OCC3)OCC2 ; COc1cc(Br)c(CCNCc2cccc3ccccc23)cc1OC ; COc1cc(CCNCc2cccc3ccccc23)cc(OC)c1Br ; COc1cc(Br)cc(OC)c1CCNCc1cccc2ccccc12 ; COc1cc(Br)cc(OC)c1CC(C)NCc1cccc2ccccc12 ; COc1cc(Cl)ccc1CCNCc1cccc2ccccc12]

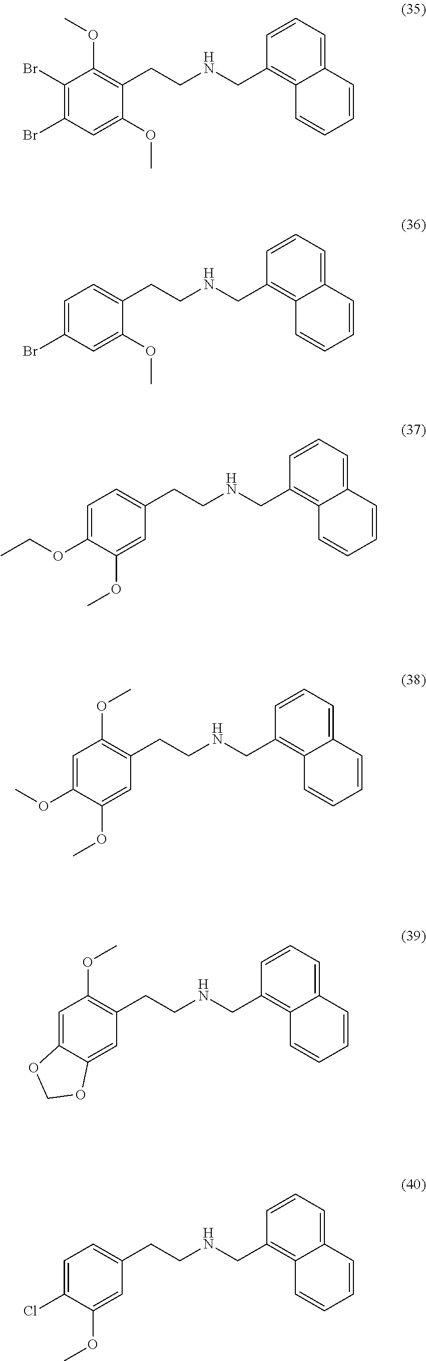 [SMILES: COc1cc(Br)c(Br)c(OC)c1CCNCc1cccc2ccccc12 ; COc1cc(Br)ccc1CCNCc1cccc2ccccc12 ; CCOc1ccc(CCNCc2cccc3ccccc23)cc1OC ; COc1cc(OC)c(OC)cc1CCNCc1cccc2ccccc12 ; COc1cc2c(cc1CCNCc1cccc3ccccc13)OCO2 ; COc1cc(CCNCc2cccc3ccccc23)ccc1Cl]

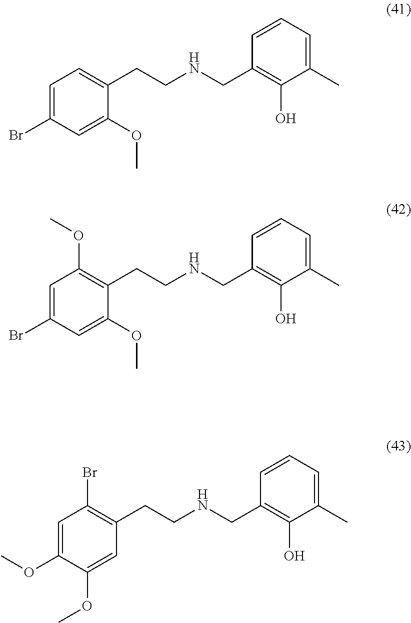

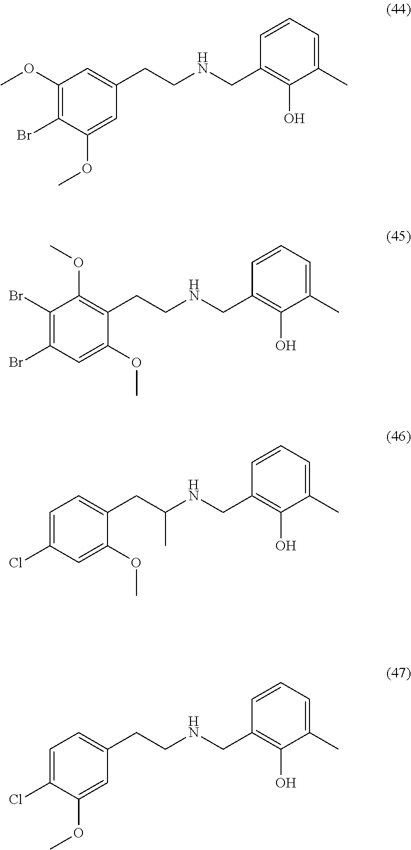

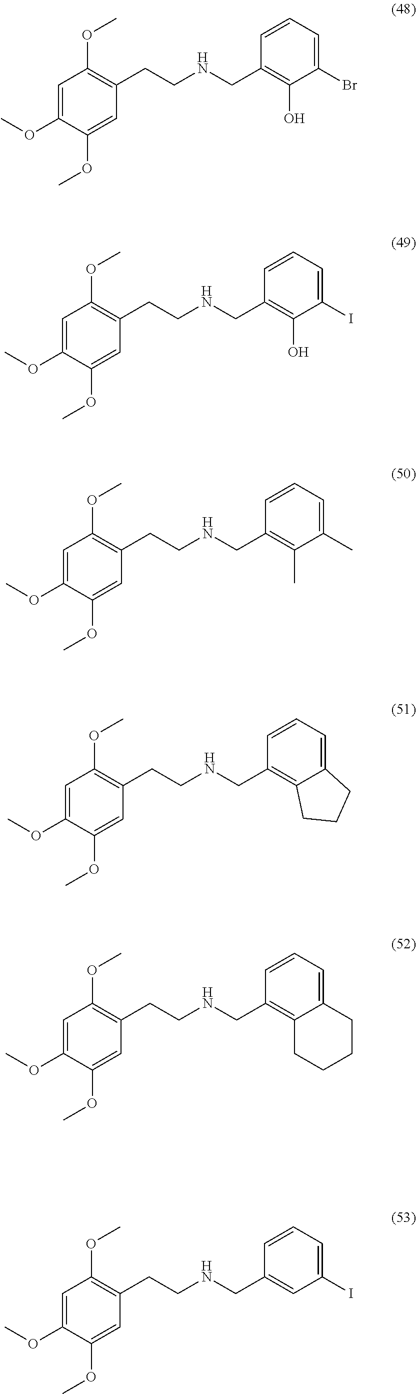

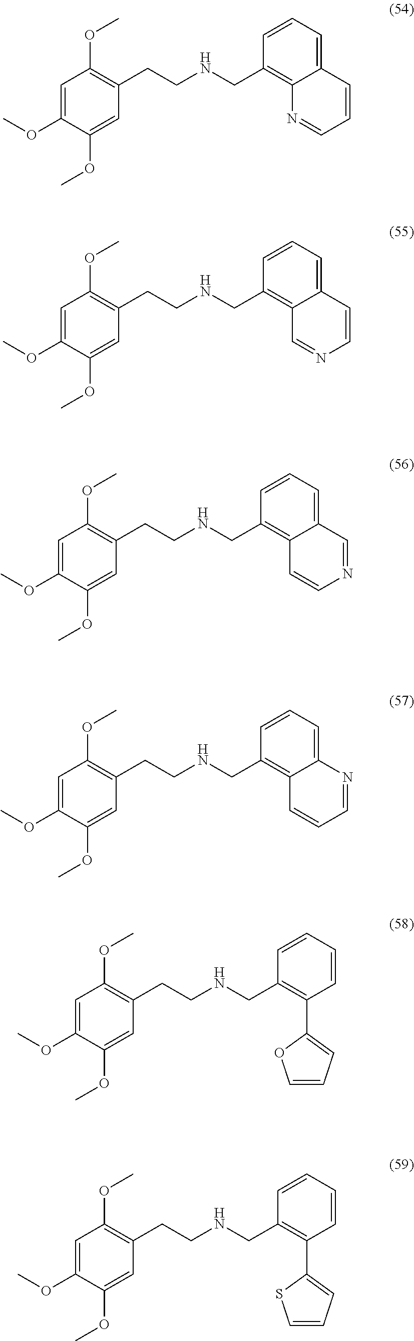 [SMILES: COc1cc(OC)c(OC)cc1CCNCc1cccc2cccnc12 ; COc1cc(OC)c(OC)cc1CCNCc1cccc2ccncc12 ; COc1cc(OC)c(OC)cc1CCNCc1cccc2cnccc12 ; COc1cc(OC)c(OC)cc1CCNCc1cccc2ncccc12 ; COc1cc(OC)c(OC)cc1CCNCc1ccccc1-c1ccco1 ; COc1cc(OC)c(OC)cc1CCNCc1ccccc1-c1cccs1]

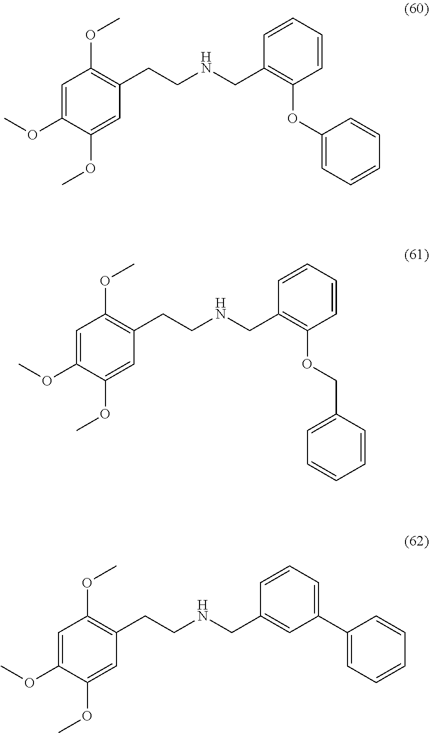 [SMILES: COc1cc(OC)c(OC)cc1CCNCc1ccccc1Oc1ccccc1 ; COc1cc(OC)c(OC)cc1CCNCc1ccccc1OCc1ccccc1 ; COc1cc(OC)c(OC)cc1CCNCc1cccc(-c2ccccc2)c1]

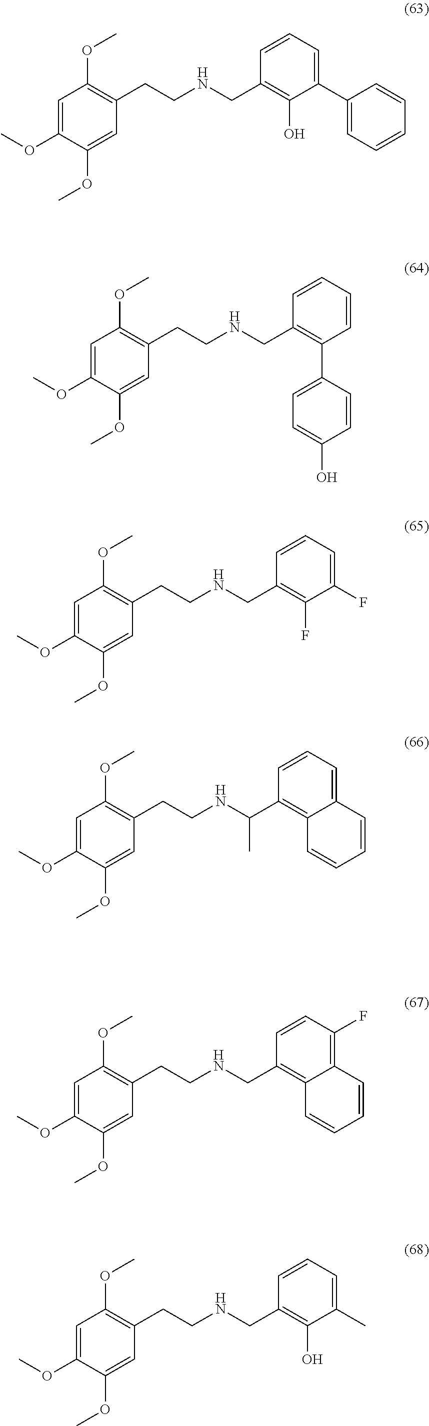

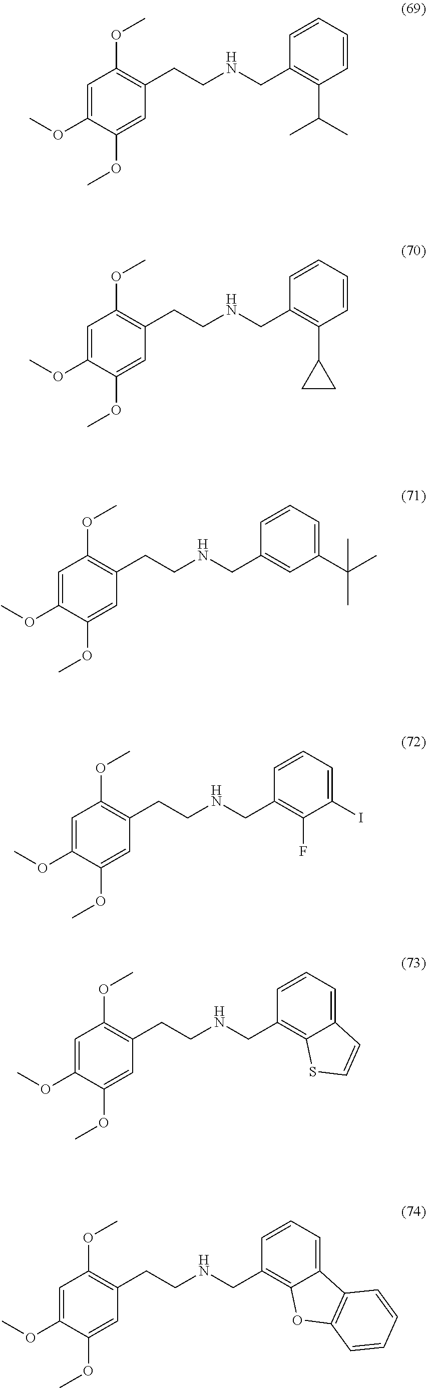

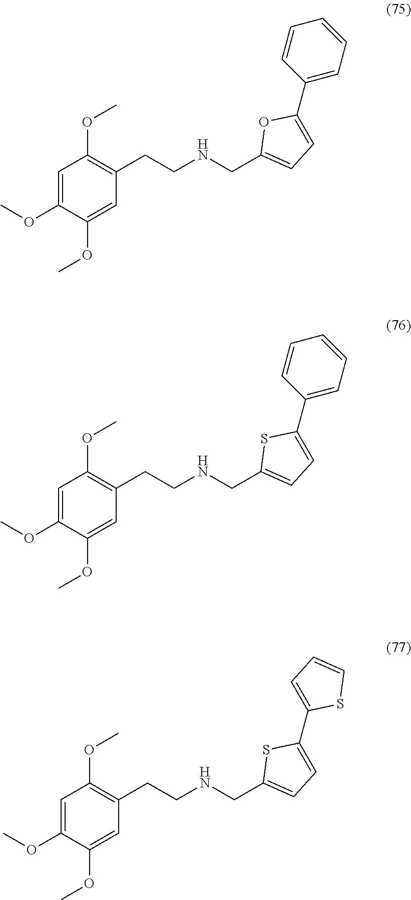 [SMILES: COc1cc(OC)c(OC)cc1CCNCc1ccc(-c2ccccc2)o1 ; COc1cc(OC)c(OC)cc1CCNCc1ccc(-c2ccccc2)s1 ; COc1cc(OC)c(OC)cc1CCNCc1ccc(-c2cccs2)s1]

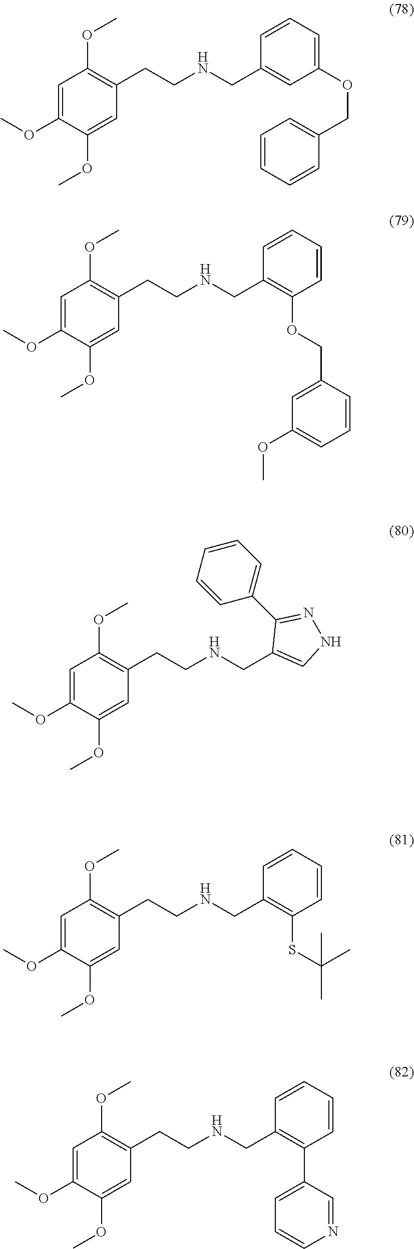

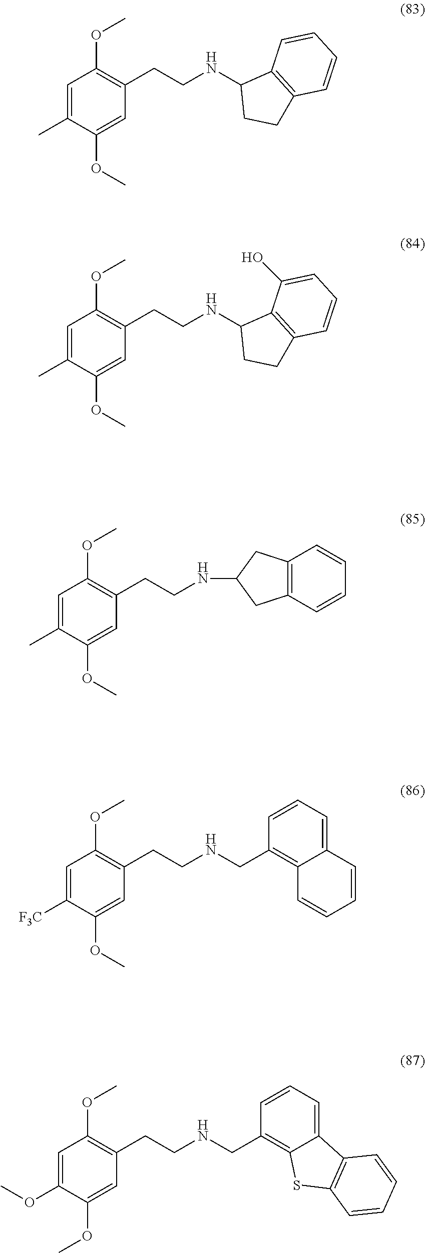 [SMILES: COc1cc(CCNC2CCc3ccccc32)c(OC)cc1C ; COc1cc(CCNC2CCc3cccc(O)c32)c(OC)cc1C ; COc1cc(CCNC2Cc3ccccc3C2)c(OC)cc1C ; COc1cc(C(F)(F)F)c(OC)cc1CCNCc1cccc2ccccc12 ; COc1cc(OC)c(OC)cc1CCNCc1cccc2c1sc1ccccc12]

Evaluation of the Binding Affinities and Functional Activities of Compounds at 5-HT₂ Subtypes

Table 2 shows the potencies of 2C—N(1) and N-substituted derivatives (25N) at 5-HT₂ subtypes, measured using Ca²⁺ flux assays. The compounds exhibited a wide range of potencies.

Table 3 shows Gq and β-arrestin BRET results. Data presented as mean±SEM (n=3). (30)-(65) reported as mean±SEM from technical replicates. Inactive refers to no detectable curve.

Receptor binding affinities at 5-HT subtypes are presented in Table 4. The compounds exhibited selectivity for 5-HT₂ subtypes over other 5-HT receptors. However, there was little selectivity for the individual 5-HT₂ subtypes, common to other 5-HT_{2A} agonists of the phenylalkylamine, lysergamide, and tryptamine scaffolds.

TABLE 2
Functional activities of 2C-N (1) and N-substituted derivatives
(25N) at 5-HT₂ subtypes, measured using Ca²⁺ flux assays.

| | 5-HT_{2A} | | 5-HT_{2B} | | 5-HT_{2C} | |
|---|---|---|---|---|---|---|
| | EC₅₀, nM | Emax | EC₅₀, nM | Emax | EC₅₀, nM | Emax |
| | (pEC₅₀ ± SEM) | % 5-HT | (pEC₅₀ ± SEM) | % 5-HT | (pEC₅₀ ± SEM) | % 5-HT |
| 5-HT | 0.42 | 100 | 1.10 | 100 | 0.22 | 100 |
| | (9.37 ± 0.03) | | (8.96 ± 0.03) | | (9.66 ± 0.02) | |
| 2C-N | 4.56 | 100 ± 1 | 50.3 | 72 ± 3 | 60.1 | 69 ± 2 |
| (1) | (8.31 ± 0.03) | | (7.30 ± 0.09) | | (7.22 ± 0.07) | |
| 25N-NB | 1.27 | 86 ± 1 | No activity | <10 | 26.2 | 75 ± 1 |
| (2) | (8.90 ± 0.04) | | | | (7.58 ± 0.05) | |
| 25N-NBOH | 0.32 | 87 ± 2 | No activity | <10 | 1.78 | 100 ± 2 |
| (3) | (9.50 ± 0.06) | | | | (8.75 ± 0.04) | |
| 25N-NBOMe | 0.32 | 94 ± 1 | No activity | <10 | 0.84 | 102 ± 1 |
| (4) | (9.50 ± 0.03) | | | | (9.07 ± 0.03) | |
| 25N-NBOEt | 0.72 | 87 ± 2 | No activity | <10 | 0.88 | 99 ± 1 |
| (5) | (9.14 ± 0.08) | | | | (9.06 ± 0.04) | |
| 25N-NBMe | 1.19 | 85 ± 1 | No activity | <10 | 33.2 | 89 ± 2 |
| (6) | (8.92 ± 0.04) | | | | (7.48 ± 0.03) | |
| 25N-NBF | 1.56 | 84 ± 1 | No activity | <10 | 28.8 | 82 ± 2 |
| (7) | (8.81 ± 0.04) | | | | (7.54 ± 0.05) | |
| 25N-NBCl | 2.03 | 80 ± 2 | No activity | <10 | 89.3 | 81 ± 2 |
| (8) | (8.69 ± 0.07) | | | | (7.05 ± 0.05) | |
| 25N-NBBr | 2.36 | 79 ± 2 | No activity | <10 | 144 | 78 ± 2 |
| (9) | (8.63 ± 0.08) | | | | (6.84 ± 0.06) | |
| 25N-NBI | 3.29 | 88 ± 1 | No activity | <10 | 682 | 45 ± 2 |
| (10) | (8.48 ± 0.05) | | | | (6.17 ± 0.08) | |
| 25N-NBOCF₂H | 1.92 | 95 ± 1 | No activity | <10 | 3.99 | 101 ± 1 |
| (11) | (8.72 ± 0.04) | | | | (8.40 ± 0.03) | |
| 25N-NBOCF₃ | 8.09 | 75 ± 2 | No activity | <10 | 58.3 | 89 ± 1 |
| (12) | (8.09 ± 0.09) | | | | (7.23 ± 0.04) | |
| 25N-NBMDF₂ | 3.02 | 86 ± 2 | No activity | <10 | 431 | 49 ± 4 |
| (13) | (8.52 ± 0.05) | | | | (6.36 ± 0.20) | |
| 25N-NBCF₃ | 12.3 | 77 ± 1 | No activity | <10 | 271 | 20 ± 2 |
| (14) | (7.91 ± 0.04) | | | | (6.57 ± 0.22) | |
| 25N-NBNO₂ | 15.6 | 64 ± 2 | No activity | <10 | 583 | 36 ± 2 |
| (15) | (7.81 ± 0.07) | | | | (6.23 ± 0.08) | |
| 25N-N-1-Nap | No activity | <10 | No activity | <10 | No activity | <10 |
| (16) | | | | | | |
| 25N-NBPh | No activity | <10 | No activity | <10 | No activity | <10 |
| (17) | | | | | | |
| 25N-NB- | 1.58 | 55 ± 2 | No activity | <10 | No activity | <10 |
| 2-OH-3-Me | (8.80 ± 0.13) | | | | | |
| (18) | | | | | | |
| 25N-NB- | 2.35 | 83 ± 1 | No activity | <10 | 74.5 | 77 ± 2 |
| 2-MeO-3-F | (8.63 ± 0.05) | | | | (7.13 ± 0.08) | |
| (19) | | | | | | |
| 25N-NB- | 133 | 72 ± 1 | No activity | <10 | No activity | <10 |
| 2,5-DiMeO | (6.88 ± 0.04) | | | | | |
| (20) | | | | | | |
| 25N-NB-3-OH | 3.32 | 55 ± 2 | No activity | <10 | 49.1 | 53 ± 4 |
| (21) | (8.48 ± 0.09) | | | | (7.31 ± 0.19) | |
| 25N-NB-3-Me | 2.27 | 54 ± 1 | No activity | <10 | 14.3 | 15 ± 2 |
| (22) | (8.64 ± 0.09) | | | | (7.84 ± 0.30) | |
| 25N-NB-4-Me | 38.3 | 42 ± 3 | No activity | <10 | 844 | 14 ± 1 |
| (23) | (7.42 ± 0.17) | | | | (6.07 ± 0.15) | |
| 25N-NB-3-F | 2.25 | 85 ± 2 | No activity | <10 | 149 | 54 ± 1 |
| (24) | (8.65 ± 0.06) | | | | (6.83 ± 0.04) | |
| 25N-NB-4-F | 19.7 | 48 ± 2 | No activity | <10 | 413 | 21 ± 2 |
| (25) | (7.71 ± 0.14) | | | | (6.38 ± 0.15) | |

5-HT₂ receptor subtype Ca²⁺ flux results. No activity Potencies could not be determined as Emax was <10% (relative to 5-HT)

TABLE 3
5-HT_{2A} in vitro G protein and arrestin functional activity of known 5-HT_{2A} agonist
psychedelics and novel compounds. No activity refers to no detectable curve.

| | | | 5-HT_{2A} β- | |
|---|---|---|---|---|
| | 5-HT_{2A} Gq | | arrestin | 5-HT_{2A} β- |
| | dissociation | 5-HT_{2A} Gq | recruitment | arrestin Emax |
| Compound | pEC50 | Emax (% 5-HT) | pEC50 | (% 5-HT) |
| 5-HT | 7.8 ± 0.08 | 100.0 | 7.74 ± 0.7 | 100.0 |
| LSD | 8.84 ± 0.09 | 95 ± 2 | 9.24 ± 0.14 | 85 ± 3 |
| DMT | 7.17 ± 0.07 | 76 ± 2 | 7.12 ± 0.13 | 64 ± 3 |
| 5-MeO-DMT | 7.76 ± 0.09 | 85 ± 3 | 7.7 ± 0.13 | 93 ± 5 |
| 2C-I | 8.83 ± 0.05 | 93.4 ± 1.4 | 8.25 ± 0.07 | 74.3 ± 1.6 |
| 2C-B | 8.92 ± 0.03 | 100.8 ± 0.9 | 8.62 ± 0.04 | 84.0 ± 1.0 |
| 2C-N | 7.88 ± 0.04 | 87.9 ± 1.4 | 7.81 ± 0.08 | 83.8 ± 2.5 |
| (±)-DOI | 8.14 ± 0.12 | 108.2 ± 4.2 | 8.66 ± 0.08 | 88.8 ± 1.9 |
| 25I-NBOMe | 9.45 ± 0.04 | 99.8 ± 1.2 | 9.28 ± 0.09 | 137.2 ± 3.7 |
| 25D-NBOMe (26) | 9.65 ± 0.05 | 93.0 ± 1.3 | 9.43 ± 0.09 | 126.0 ± 3.4 |
| 25N-NB (2) | 8.70 ± 0.06 | 79.5 ± 1.5 | 8.96 ± 0.11 | 115.1 ± 4.1 |
| 25N-NBOMe (4) | 9.59 ± 0.06 | 98 ± 0.9 | 9.66 ± 0.09 | 136.5 ± 3.8 |
| 25N-NB-2-OH-3-Me (18) | 9.49 ± 0.11 | 50.8 ± 1.6 | 9.43 ± 0.10 | 130.7 ± 4.0 |
| 25N-NBPh (17) | 7.77 ± 0.18 | 23.2 ± 1.5 | 8.39 ± 0.08 | 109.4 ± 2.8 |
| 25N-N1-Nap (16) | 8.68 ± 0.19 | 23.7 ± 1.4 | 9.39 ± 0.08 | 78.2 ± 1.8 |
| 25N-NB-2-MeO-3-F (19) | 8.56 ± 0.08 | 71.1 ± 1.9 | 8.33 ± 0.08 | 120.4 ± 3.3 |
| 25N-NB-3-Me (22) | 8.64 ± 0.25 | 41.7 ± 3.4 | 9.08 ± 0.14 | 94.7 ± 4.2 |
| 25N-NB-3-OH (21) | 7.94 ± 0.23 | 49.8 ± 4.3 | 8.36 ± 0.13 | 118.6 ± 5.2 |
| 25D-N1-Nap (27) | 8.16 ± 0.24 | 34.3 ± 2.9 | 8.86 ± 0.10 | 75.5 ± 2.4 |
| 25D-NBPh (28) | 6.09 ± 0.16 | 23.2 ± 4.8 | 7.64 ± 0.12 | 107 ± 4.7 |
| (29) | 8.06 ± 0.12 | 31.8 ± 1.4 | 8.99 ± 0.10 | 62.5 ± 2.3 |
| (30) | 7.37 ± 0.13 | 32.3 ± 1.6 | 8.35 ± 0.12 | 65.1 ± 3.0 |
| (31) | 6.77 ± 0.21 | 20.3 ± 1.9 | 7.45 ± 0.18 | 50.8 ± 3.4 |
| (32) | 8.41 ± 0.11 | 33.0 ± 1.1 | 8.98 ± 0.07 | 85.5 ± 2.0 |
| (33) | 7.50 ± 0.08 | 36.6 ± 1.1 | 8.31 ± 0.08 | 98.3 ± 3.3 |
| (34) | 7.43 ± 0.21 | 25.2 ± 2.0 | 8.51 ± 0.14 | 83.6 ± 3.6 |
| (35) | 8.41 ± 0.16 | 30.2 ± 1.5 | 8.71 ± 0.09 | 84.5 ± ±2.3 |
| (36) | 8.12 ± 0.13 | 26.3 ± 1.2 | 8.59 ± 0.12 | 80.2 ± 3.1 |
| (37) | 6.30 ± 0.12 | 29.3 ± 1.9 | 7.33 ± 0.08 | 75.4 ± 2.4 |
| (38) | 8.26 ± 0.14 | 24.3 ± 1.2 | 8.79 ± 0.08 | 74.9 ± 1.9 |
| (39) | 7.81 ± 0.20 | 26.4 ± 2.0 | 8.23 ± 0.09 | 80.1 ± 2.5 |
| (40) | 6.76 ± 0.13 | 29.9 ± 1.8 | 7.34 ± 0.12 | 80.8 ± 3.8 |
| (41) | 8.65 ± 0.06 | 68.7 ± 1.3 | 8.60 ± 0.14 | 86.6 ± 3.9 |
| (42) | 8.96 ± 0.07 | 69.2 ± 1.5 | 9.29 ± 0.16 | 78.9 ± 3.7 |
| (43) | 8.19 ± 0.06 | 65.4 ± 1.4 | 8.20 ± 0.11 | 91.7 ± 3.4 |
| (44) | 7.92 ± 0.05 | 70.4 ± 1.2 | 7.95 ± 0.11 | 66.1 ± 2.8 |
| (45) | 8.65 ± 0.09 | 70.5 ± 2.0 | 8.88 ± 0.24 | 77.5 ± 6.0 |
| (46) | 8.32 ± 0.07 | 67.8 ± 1.6 | 8.95 ± 0.24 | 72.9 ± 5.6 |
| (47) | 7.51 ± 0.07 | 66.9 ± 1.8 | 8.16 ± 0.11 | 80.5 ± 3.2 |
| (48) | 7.69 ± 0.09 | 53.6 ± 1.7 | 8.09 ± 0.16 | 85.0 ± 4.8 |
| (49) | 7.84 ± 0.08 | 52.8 ± 1.5 | 7.93 ± 0.19 | 80.5 ± 5.5 |
| (50) | 8.46 ± 0.14 | 23.6 ± 1.0 | 8.24 ± 0.21 | 73.9 ± 5.4 |
| (51) | 8.77 ± 0.14 | 41.2 ± 1.9 | 8.48 ± 0.18 | 72.9 ± 4.2 |
| (52) | 8.12 ± 0.07 | 43.7 ± 1.1 | 8.75 ± 0.24 | 65.0 ± 4.9 |
| (53) | 7.68 ± 0.10 | 50.1 ± 1.9 | 7.95 ± 0.18 | 82.7 ± 5.4 |
| (54) | 7.95 ± 0.07 | 54.3 ± 1.5 | 8.07 ± 0.10 | 100.4 ± 3.5 |
| (56) | 7.58 ± 0.06 | 64.1 ± 1.5 | 7.76 ± 0.40 | 75.8 ± 8.3 |
| (57) | 5.94 ± 0.18 | 34.2 ± 4.0 | 6.14 ± 0.27 | 92.0 ± 8.9 |
| (58) | 7.30 ± 0.11 | 51.9 ± 2.2 | 7.25 ± 0.14 | 112.0 ± 6.2 |
| (59) | 7.62 ± 0.12 | 34.9 ± 1.6 | 8.07 ± 0.10 | 100.4 ± 3.5 |
| (60) | 6.86 ± 0.12 | 63.3 ± 3.5 | 6.99 ± 0.20 | 117.0 ± 14.6 |
| (61) | No activity | <10% | 8.48 ± 0.18 | 72.9 ± 4.2 |
| (62) | 6.74 ± 0.19 | 23.7 ± 2.1 | 7.25 ± 0.14 | 112.0 ± 6.2 |
| (63) | No activity | <10% | 6.99 ± 0.20 | 117.0 ± 14.6 |
| (64) | 6.21 ± 0.11 | 43.6 ± 2.5 | 7.93 ± 0.19 | 80.5 ± 5.5 |
| (65) | 7.19 ± 0.07 | 76.5 ± 2.3 | 6.77 ± 0.06 | 101.2 ± 2.7 |
| (67) | 7.64 ± 0.10 | 41.0 ± 1.3 | 7.99 ± 0.13 | 73.6 ± 3.2 |
| (68) | 8.49 ± 0.04 | 63.4 ± 0.8 | 8.85 ± 0.08 | 89.1 ± 2.3 |
| (70) | 8.44 ± 0.05 | 97.5 ± 1.5 | 8.16 ± 0.11 | 116.1 ± 4.4 |
| (71) | 5.79 ± 0.18 | 38.6 ± 5.0 | 5.79 ± 0.15 | 60.4 ± 5.2 |
| (72) | 7.47 ± 0.08 | 60.7 ± 1.7 | 8.15 ± 0.14 | 63.5 ± 3.2 |
| (74) | 7.09 ± 0.13 | 40.2 ± 1.9 | 7.79 ± 0.15 | 68.3 ± 3.4 |
| (75) | No activity | <10% | 6.05 ± 0.18 | 50.3 ± 4.7 |
| (76) | 5.97 ± 0.15 | 57.4 ± 4.6 | 7.04 ± 0.13 | 78.0 ± 4.0 |
| (77) | 5.67 ± 0.17 | 49.0 ± 5.1 | 6.29 ± 0.10 | 82.5 ± 4.2 |
| (78) | 5.53 ± 0.21 | 32.8 ± 4.5 | 6.68 ± 0.19 | 50.3 ± 4.1 |
| (79) | No activity | <10% | 5.95 ± 0.27 | 33.0 ± 4.8 |
| (80) | 6.27 ± 0.15 | 31.8 ± 2.3 | 7.10 ± 0.09 | 101.7 ± 3.7 |
| (83) | 8.20 ± 0.40 | 50.7 ± 7.0 | 8.09 ± 0.29 | 111.8 ± 11.5 |
| (84) | 8.57 ± 0.21 | 71.91 ± 4.9 | 8.46 ± 0.12 | 113.1 ± 4.3 |
| (85) | 7.75 ± 0.22 | 53.5 ± 4.3 | 8.29 ± 0.21 | 70.9 ± 5.1 |

TABLE 4
Data presented as mean ± SEM (n = 3). (30)-(65) reported as
mean ± SEM from technical replicates. No activity refers to no detectable curve.

| | 5-HT_{2B} | | 5-HT_{2C} | |
|---|---|---|---|---|
| | Gq Dissociation (BRET) | | Gq Dissociation (BRET) | |
| | | E_{MAX} ± SEM | | E_{MAX} ± SEM |
| Compound | pEC50 ± SEM | (%5-HT) | pEC50 ± SEM | (%5-HT) |
| 2C-B | 7.90 ± 0.04 | 97.4 ± 1.4 | 9.20 ± 0.05 | 97.8 ± 1.5 |
| 2C-I | 7.72 ± 0.07 | 101.0 ± 2.1 | 9.34 ± 0.05 | 106.9 ± 1.5 |
| 2C-N | 7.13 ± 0.08 | 87.7 ± 2.8 | 7.90 ± 0.09 | 78.4 ± 2.5 |
| 25I-NBOMe | 8.47 ± 0.10 | 78.6 ± 2.6 | 10.01 ± 0.09 | 123.8 ± 3.1 |
| DOI | 7.90 ± 0.09 | 103.2 ± 2.8 | 9.08 ± 0.11 | 114.3 ± 3.4 |
| 25N-NB (2) | 7.42 ± 0.19 | 32.6 ± 2.3 | 8.07 ± 0.07 | 89.4 ± 2.3 |
| 25N-NBOMe (4) | 8.41 ± 0.11 | 61.8 ± 2.3 | 9.52 ± 0.09 | 111.7 ± 2.8 |
| 25N-NBMe (6) | 8.02 ± 0.44 | 22.2 ± 3.2 | 7.94 ± 0.07 | 89.4 ± 2.3 |
| 25N-NBF (7) | 7.12 ± 0.27 | 24.8 ± 3.2 | 7.85 ± 0.07 | 83.1 ± 2.1 |
| 25N-NBCl (8) | 6.69 ± 0.25 | 27.0 ± 3.0 | 7.67 ± 0.10 | 82.5 ± 2.8 |
| 25N-NBBr (9) | 6.61 ± 0.28 | 17.0 ± 2.1 | 7.51 ± 0.08 | 81.0 ± 2.3 |
| 25N-NBI (10) | 7.53 ± 0.31 | 21.8 ± 2.3 | 7.06 ± 0.07 | 71.5 ± 2.2 |
| 25N-NBNO₂ (15) | No activity | No activity | 6.83 ± 0.17 | 61.3 ± 4.4 |
| 25N-NB-2- | No activity | No activity | 8.67 ± 0.17 | 45.8 ± 2.4 |
| OH-3-Me (18) | | | | |
| 25N-N1-Nap (16) | No activity | No activity | 7.73 ± 0.35 | 34.1 ± 4.4 |
| 25N-NBPh (17) | 6.50 ± 0.25 | 47.0 ± 5.5 | 6.19 ± 0.19 | 96.9 ± 10.0 |
| (29) | No activity | No activity | 8.38 ± 0.08 | 78.1 ± 2.1 |
| (30) | No activity | No activity | 7.18 ± 0.10 | 81.4 ± 3.4 |
| (31) | No activity | No activity | 7.38 ± 0.07 | 80.2 ± 2.2 |
| (32) | No activity | No activity | 8.20 ± 0.06 | 81.0 ± 1.6 |
| (33) | No activity | No activity | 7.19 ± 0.07 | 80.3 ± 2.4 |
| (34) | No activity | No activity | 7.42 ± 0.06 | 75.9 ± 1.7 |
| (35) | No activity | No activity | 8.26 ± 0.06 | 69.8 ± 1.3 |
| (36) | No activity | No activity | 7.61 ± 0.06 | 76.0 ± 1.8 |
| (37) | No activity | No activity | 6.58 ± 0.07 | 84.5 ± 2.8 |
| (38) | No activity | No activity | 7.90 ± 0.09 | 64.4 ± 2.0 |
| (39) | No activity | No activity | 7.77 ± 0.07 | 90.5 ± 2.3 |
| (40) | No activity | No activity | 7.01 ± 0.05 | 87.8 ± 2.0 |
| (41) | No activity | No activity | 8.31 ± 0.07 | 72.2 ± 1.6 |
| (42) | No activity | No activity | 8.82 ± 0.06 | 78.1 ± 1.6 |
| (43) | No activity | No activity | 7.68 ± 0.07 | 75.4 ± 2.0 |
| (44) | No activity | No activity | 8.15 ± 0.07 | 80.2 ± 2.1 |
| (45) | No activity | No activity | 8.82 ± 0.08 | 81.0 ± 2.1 |
| (46) | No activity | No activity | 7.94 ± 0.09 | 95.5 ± 3.2 |
| (47) | No activity | No activity | 7.71 ± 0.09 | 98.0 ± 3.1 |
| (48) | No activity | No activity | 6.76 ± 0.19 | 69.2 ± 6.1 |
| (49) | No activity | No activity | 7.57 ± 0.09 | 93.5 ± 3.1 |
| (50) | No activity | No activity | 8.81 ± 0.28 | 30.4 ± 2.7 |
| (51) | No activity | No activity | 7.03 ± 0.23 | 52.5 ± 5.2 |
| (52) | No activity | No activity | 7.46 ± 0.23 | 43.6 ± 3.8 |
| (53) | 9.14 ± 0.46 | 20.3 ± 2.8 | 7.82 ± 0.10 | 91.5 ± 3.2 |
| (54) | No activity | No activity | 7.83 ± 0.09 | 90.5 ± 3.1 |
| (56) | No activity | No activity | 6.90 ± 0.09 | 80.9 ± 3.4 |
| (57) | No activity | No activity | 6.70 ± 0.09 | 77.6 ± 3.2 |
| (58) | 8.51 ± 0.17 | 37.6 ± 2.0 | 7.85 ± 0.11 | 94.0 ± 4.0 |
| (59) | No Activity | No activity | No Activity | No Activity |
| (60) | No activity | No activity | 5.77 ± 0.15 | 113.1 ± 11.6 |
| (61) | No activity | No activity | No Activity | No Activity |
| (54) | No activity | No activity | 7.83 ± 0.09 | 90.5 ± 3.1 |
| (62) | No activity | No activity | 6.66 ± 0.16 | 72.8 ± 5.2 |
| (63) | No activity | No activity | 7.29 ± 0.08 | 79.2 ± 2.6 |
| (64) | 5.52 ± 0.49 | 29.2 ± 10.6 | 5.77 ± 0.18 | 80.6 ± 9.8 |
| (65) | No activity | No activity | 6.70 ± 0.17 | 83.3 ± 6.3 |

TABLE 5
5-HT receptor subtype pKi affinities.

| | 5-HT Receptors (Kᵢ ± SEM) | | | | | | | | | | |
|---|---|---|---|---|---|---|---|---|---|---|---|
| Compound | 5-HT_{1A} | 5-HT_{1B} | 5-HT_{1D} | 5-HT₁ₑ | 5-HT_{2A} | 5-HT_{2B} | 5-HT_{2C} | 5-HT₃ | 5-HT₅ₐ | 5-HT₆ | 5-HT₇ |
| 2C-N | 5.84 ± | <5 | 6.08 ± | 6.17 ± | 7.14 ± | 6.91 ± | 6.79 ± | <5 | <5 | 6.60 ± | <5 |
| (1) | 0.18 | | 0.09 | 0.13 | 0.02 | 0.03 | 0.18 | | | 0.18 | |
| 25N-NBOMe | 5.73 ± | <5 | 5.25 ± | <5 | 9.26 ± | 8.35 ± | 8.16 ± | <5 | <5 | 7.26 ± | <5 |
| (4) | 0.15 | | 0.06 | | 0.15 | 0.08 | 0.07 | | | 0.13 | |
| 25N-NBI | 6.00 ± | <5 | <5 | <5 | 8.27 ± | 7.70 ± | 7.36 ± | <5 | <5 | 6.42 ± | <5 |
| (10) | 0.06 | | | | 0.08 | 0.17 | 0.02 | | | 0.04 | |
| 25N-NBOCF₃ | 5.95 ± | <5 | <5 | <5 | 7.84 ± | 7.86 ± | 7.33 ± | <5 | 5.80 ± | 6.12 ± | <5 |
| (12) | 0.08 | | | | 0.14 | 0.12 | 0.08 | | 0.10 | 0.09 | |
| 25N-NBMDF₂ | 6.00 | <5 | <5 | <5 | 8.92 | 7.33 | 6.67 | <5 | ND | ND | ND |
| (13) | | | | | | | | | | | |
| 25N-NBCF₃ | 5.97 ± | <5 | <5 | <5 | 7.70 ± | 7.23 ± | 6.99 ± | <5 | <5 | 6.39 ± | <5 |
| (14) | 0.05 | | | | 0.05 | 0.22 | 0.08 | | | 0.02 | |
| 25N-NBNO₂ | 5.71 ± | <5 | <5 | <5 | 7.09 ± | 6.72 ± | 6.55 ± | <5 | <5 | <5 | <5 |
| (15) | 0.15 | | | | 0.04 | 0.05 | 0.06 | | | | |
| 25N-N-1-Nap | 6.62 ± | <5 | 6.06 ± | <5 | 8.94 ± | 8.93 ± | 8.37 ± | <5 | 5.96 ± | 7.10 ± | 6.73 ± |
| (16) | 0.02 | | 0.06 | | 0.14 | 0.27 | 0.06 | | 0.16 | 0.11 | 0.10 |
| 25N-NBPh | 5.75 | <5 | 5.92 | <5 | 9.48 | 5.85 | 6.43 | <5 | ND | ND | ND |
| (17) | | | | | | | | | | | |
| 25N-NB-2- | 6.73 ± | <5 | 5.89 ± | <5 | 9.32 ± | 9.11 ± | 8.54 ± | <5 | 6.30 ± | 6.68 ± | 6.06 ± |
| OH-3-Me | 0.10 | | 0.08 | | 0.09 | 0.14 | 0.02 | | 0.08 | 0.02 | 0.04 |
| (18) | | | | | | | | | | | |
| 25N-NB-2- | 6.00 ± | <5 | <5 | <5 | 8.13 ± | 7.60 ± | 7.39 ± | <5 | 5.54 ± | <5 | <5 |
| MeO-3-F | 0.12 | | | | 0.05 | 0.17 | 0.04 | | 0.02 | | |
| (19) | | | | | | | | | | | |
| 25N-NB- | 6.29 ± | <5 | <5 | <5 | 6.64 ± | 6.87 ± | 6.84 ± | <5 | <5 | <5 | <5 |
| 2,5-DiMeO | 0.07 | | | | 0.05 | 0.12 | 0.04 | | | | |
| (20) | | | | | | | | | | | |
| 25N-NB-3-OH | 5.73 ± | <5 | <5 | <5 | 7.84 ± | 7.45 ± | 7.08 ± | <5 | <5 | 7.16 ± | <5 |
| (21) | 0.10 | | | | 0.08 | 0.09 | 0.05 | | | 0.10 | |
| 25N-NB-3-Me | 5.75 | <5 | 6.03 | <5 | 9.68 | 8.54 | 7.40 | <5 | ND | ND | ND |
| (22) | | | | | | | | | | | |
| 25N-NB-4-Me | 5.58 | <5 | <5 | <5 | 8.26 | 7.28 | 6.20 | <5 | ND | ND | ND |
| (23) | | | | | | | | | | | |
| 25N-NB-3-F | <5 | <5 | <5 | <5 | 8.52 | 7.37 | 6.65 | <5 | ND | ND | ND |
| (24) | | | | | | | | | | | |
| 25N-NB-4-F | <5 | <5 | <5 | <5 | 8.21 | 7.07 | 6.09 | <5 | ND | ND | ND |
| (25) | | | | | | | | | | | |

TABLE 6
5-HT_{2A} Receptor Binding for Select Compounds using
[³H]-ketanserin displacement (n = 2).

| | 5-HT_{2A} pKᵢ |
|---|---|
| | Mean ± SEM |
| Compound | (nM) |
| Ketanserin | 8.43 ± 0.06 |
| 25N-NBOMe (4) | 8.85 ± 0.3 |
| 25N-N1-Nap (16) | 8.37 ± 0.25 |
| (67) | 7.30 ± 0.06 |
| (68) | 7.51 ± 0.11 |
| (70) | 7.15 ± 0.10 |
| (71) | 6.42 ± 0.03 |
| (72) | 7.31 ± 0.01 |
| (73) | 7.53 ± 0.10 |
| (74) | 7.17 ± 0.10 |
| (75) | 6.51 ± 0.04 |
| (76) | 6.35 ± 0.01 |
| (77) | 6.49 ± 0.00 |
| (78) | 6.60 ± 0.00 |
| (79) | 6.80 ± 0.07 |
| (80) | 6.52 ± 0.13 |

In contrast to known psychedelics, a range of efficacies (Eₘₐₓ) were observed at Gq (BRET), Gq/11 mediated Ca²⁺ mobilization, and β-arrestin recruitment (BRET) for 5-HT_{2A} (Table 2 and 3). As a whole the 25N series tended to show slightly higher efficacies towards β-arrestin or balanced activity for both signaling pathways. Unexpectedly it was found that compounds with an electron withdrawing group in the 2-position like 25N—NBOCF₂H (11), 25N—NBCF₃ (14), and 25N—NBNO₂ (15), had an Emax lower in the Gq pathway (Ca²⁺ flux and/or BRET) than full agonists compounds which contained electron donating groups (through resonance for example using op Hammett substitution constants with a value of >0.3) like 25N—NBOH (3), 25N—NBOMe (4), and 25N—NBMe (6) and lacked a HTR in mice. Other notable and unexpected findings were 25N—N-1-Nap (16), and 25N—NBPh (17). Both agents exhibited strong functional selectivity for β-arrestin (BRET) over Gq (BRET and Ca²⁺ mobilization). Compounds with a 3-substitution like 25N—NB-3-OH (21) were also 5-HT_{2A} Gq partial agonists and inactive in the HTR. 2,3-diusubstituted compounds also exhibited this reduced Emax in the G protein pathway; 25N—NB-2-OH-3-Me (18) is also notable as it is a Gq partial agonist (BRET and Ca²⁺ mobilization) but shows a super agonist (>100%) β-arrestin response (Emax=131%) in the BRET assay; this is a difference in Emax of 81%. This novel and unexpected SAR was then applied to additional phenethylamines (compounds (29)-(87) yielding compounds which retained the desired pharmacological profiles.

Agonists exhibiting partial G protein efficacy and/or a signaling bias have therapeutic potential. For example, the recently approved analgesic oliceridine is a G protein-biased MOR ligand. With respect to 5-HT_{2A}, it has been hypothesized that uncharacterized signaling bias may explain differences in the anti-inflammatory effects of some 5-HT_{2A} agonists. It has long been speculated that it is possible to separate clinically desirable effects including in psychiatric and neurological indications from the psychedelic and hallucinogenic effects of 5-HT_{2A} agonists. Non-hallucinogenic 5-HT_{2A} ligands have recently been described that can cause desirable effects in preclinical assays but do not induce the HTR in mice. However, many of these agents are tryptamines or related indolic structures which are likely to have poor selecitivty. The nature of the lack of HTR activity in these cases is also unclear and such compounds may just be 5-HT_{2A} antagonists or inverse agonists or acting through other receptor systems. 25N—NB-2-OH-3-Me (18), 25N—NB-1-Nap (16), and 25N—NBPh (17) and other analogs described (e.g., (38), (39), (58), (61), etc) are thus of high importance as they are anticipated to exhibit desirable pharmacology with unique clinical potential. The understanding of the role of G protein signaling in the psychedelic action can allow the signaling response to be optimized to retained desired clinical activity while minimzing or reducing the psychedelic action associated with 5-HT_{2A} full agonists entirely.

### 5-HT₂ Receptor Subtype Functional selectivity

Members of the 25N series and the other compounds evaluated showed high selectivity for 5-HT_{2A} over 5-HT_{2B} (Table 2 and 3 and 3.X). Based on binding affinities (Ki), these compounds are anticipated to act as 5-HT_{2B} antagonists and this was confirmed in antagonist mode screening in vitro. 5-HT_{2B} agonism is a common anti-target in drug development due to its strongly supported role in drug-induced pulmonary hypertension and cardiac valvulopathy. Most known 5-HT_{2A} agonists, including LSD, 2C—B, DMT, psilocin, have efficacy at all three 5-HT₂ subtypes: which we also observed (Table 3). 5-HT_{2B} agonism has been proposed as a potential confound associated with repeated administration of 5-HT_{2A} agonists, such as would occur with the so-called “microdosing” of LSD and psilocybin. It is also an issue with other tryptamine based non-hallucinogenic compounds. Thus compounds which are non-hallucinogenic 5-HT_{2A} receptor ligands that do not activate 5-HT_{2B} like 25N—NB-2-OH-3-Me (18) and 25N—N1-Nap (16) are of great clinical significance. Similarly, with respect to Ca²⁺ mobilization, several of members of the series (e.g., 25N-N1-Nap (16) and 25N—NB-2-OH-3-Me (18)) exhibited functional selectivity for 5-HT_{2A} over 5-HT_{2C}. Functionally selective ligands represent valuable pharmacological tools to elucidate the relative roles of the 5-HT₂ receptor subtypes, and may have unique therapeutic utility by minimizing unnecessary off-target effects or even through blocking these receptors as weak partial agonists or antagonists. Some members of the current series such as 25N—N-Nap (16) and 25N—NB-2-OH-3-Me (18) and related compounds, described may be less likely to provoke toxicity compared to existing agents.

The 5-HT_{2A} Gq partial agonist 25N—NB-2-OH-3-Me (18) lacked detectable Ca²⁺ flux responses at both 5-HT_{2B} and 5-HT_{2C}. Similar results were seen with 25N—N1-Nap (16). Similar results were seen with BRET (Table 3.X). Consistant with literature, the existing psychedelics tested acted as agonists at all three 5-HT₂ subtypes in the Gq BRET assay and show spotent and strong activity in 5-HT_{2B} (Table 3 and 3.X). This unexpected selectivity, most notable against 5-HT_{2B}, but also in a number of cases against 5-HT_{2C}, is believed to result from the substitution patterns on the phenethylamine ring (e.g., 25N—, 2,4,5-trimethoxy bezene, 2-methoxy-3,4-methylenedioxybenzene): substitutions on the N-benzyl ring (bulky 2-position substitutions for example 2-phenyl, 2-thiophenyl-, 2-O-benzyl-, 2—O-phenyl-): 2,3-disubstitutions (e.g., 25N—NB-2-OH-3-Me (18), (41)-(52)): 3- substitution (e.g., 25N—NB-3-Me (22), (53), (62)), and 4-substitution (e.g., 25N—NB-4-Me (23), 25N—NB-4-F (25)) and bulky biaryl systems in these compounds (e.g., naphthalene (e.g., 25N—N1-Nap (16), (29)-(40), quinoline, and isoquinoline ((54)-(57)) and combinations thereof.

TABLE 7
ΔLog(Eₘₐₓ/EC₅₀) Selectivity for 2A/2C for Gq/11
Ca²⁺ Mobilization Response. A higher number illustrates
responses skewed towards 5-HT_{2A}.

| | 5-HT_{2A}/5-HT_{2c} |
|---|---|
| | Selectivity Factor |
| Compound | ΔLog(Eₘₐₓ/EC₅₀) |
| 5-HT | 0.6 |
| 2C-N (1) | 19.1 |
| 25N-NB (2) | 23.6 |
| 25N-NBOH (3) | 4.9 |
| 25N-NBOMe (4) | 2.4 |
| 25N-NBOEt (5) | 1.0 |
| 25N-NBMe (6) | 26.7 |
| 25N-NBF (7) | 18.9 |
| 25N-NBCl (8) | 43.3 |
| 25N-NBBr (9) | 61.9 |
| 25N-NBI (10) | 406 |
| 25N-NBOCF₂H (11) | 1.9 |
| 25N-NBOCF₃ (12) | 6.1 |
| 25N-NBMDF₂ (13) | 252.5 |
| 25N-NBCF₃ (14) | 85.5 |
| 25N-NBNO₂ (15) | 66.8 |
| 25N-N-1-Nap (16) | 5-HT_{2A} Selective |
| 25N-NBPh (17) | 5-HT_{2A} Selective |
| 25N-NB-2-OH-3-Me (18) | 5-HT_{2A} Selective |
| 25N-NB-2-MeO-3-F (19) | 33.8 |
| 25N-NB-2,5-DiMeO (20) | 5-HT_{2A} Selective |
| 25N-NB-3-OH (21) | 92.6 |
| 25N-NB-3-Me (22) | 29.1 |
| 25N-NB-4-Me (23) | 66.1 |
| 25N-NB-3-F (24) | 154.4 |
| 25N-NB-4-F (25) | 43.3 |

### In Vivo Activity in Mice

HTR in mice is a commonly used rodent behavioral proxy for psychedelic activity in humans and is believed to be mediated by 5-HT_{2A} receptor activation. Importantly, non-hallucinogenic 5-HT_{2A} agonists such as lisuride do not induce the HTR (González-Maeso et al. (2003) Transcriptome fingerprints distinguish hallucinogenic and nonhallucinogenic 5-hydroxytryptamine 2A receptor agonist effects in mouse somatosensory cortex. J Neurosci 23: 8836-8843). For a large series of structurally diverse hallucinogens, a robust correlation was recently observed between HTR activity in mice and human psychedelic potency (Halberstadt et al. (2020) Correlation between the potency of hallucinogens in the mouse head-twitch response assay and their behavioral and subjective effects in other species. Neuropharmacology 167: 107933). Similar correltions are also observed between HTR activity and potency in the rat drug descrimination assay, which is another rodent paradigm used to assess the behavioral response to psychedelic drugs. Seventeen 25N derivatives were selected for HTR dose-response experiments. When tested in male C57BL/6J mice, eleven of the compounds increased HTR counts over baseline levels (Table 6). Consistent with its potent ED₅₀ (0.11 mg/kg) in the HTR, 25N—NBOMe (4) acts as a potent hallucinogen in humans and is active at doses of ˜0.3-0.8 mg when administered by the intranasal or sublingual routes. The magnitude of the HTR observed (counts above baseline levels during a 30-minute assessment) is significantly and robustly correlated with the Emax observed in 5-HT_{2A} Gq BRET assay and a Ca²⁺ mobilization assay. However, a significant correlation was not observed with 5-HT_{2A} Emax in the beta-arrestin assay (BRET). This finding is unexpected and in contrast to claims in the literature that arrestin activation is responsible for the psychedelic effects of 5-HT_{2A} agonists. Indeed, we have found that the ability of the 5-HT_{2A} agonists (+/−)-DOI and 25N—NBOMe to induce the HTR in C57BL/6J mice can be blocked by pretreatment with YM-254,890 or edelfosine, drugs that inhibit Gq/11 or phospholipase C, respectively. Pretreatment of mice with 25N—NB-1-Nap (16), 25N—NBPh (17), and 25N—NB-2-OH-3-Me (18) blocked the HTR induced by the 5-HT_{2A} agonist and psychedelic amphetamine DOI (FIG. 4), confirming their ability to cross the blood brain barrier and interact with 5-HT_{2A} in vivo. Similarly, in BRET experiments, 25N—NBPh (17) and 25N—NB-1-Nap (16) antagonized 5-HT-induced 5-HT_{2A}-mediated Gq signaling. Thus, there appears to be a threshold level of efficacy required in the 5-HT_{2A} G protein pathway for inducing HTR and intense hallucinogenic effects in humans. The existance of an activity threshold can also explain why weak 5-HT_{2A} partial agonists such as lisuride (a non-psychedelic LSD analog) fail to induce the HTR. According to Cuassac et al. (2008), lisuride (Emax=48.6%) has substantially lower efficacy than LSD (Eₘₐₓ=84.6%) or DOI (Emax=81.3%) in a 5-HT_{2A}-Gq calcium flux assay (Cussac et al. (2008) Agonist-directed trafficking of signalling at serotonin 5-HT_{2A}, 5-HT_{2B} and 5-HT2C—VSV receptors mediated Gq/11 activation and calcium mobilisation in CHO cells. Eur J Pharmacol vol. 594, pp. 32-38). 5-HT_{2A} ligands with relative efficacy at 5-HT_{2A} below the G protein threshold do not induce the HTR in mice and will show reduced intensity, a mild psychedelic or hallucinogenic effect or a complete absence of acute hallucinogenic effects, similar to lisuride.

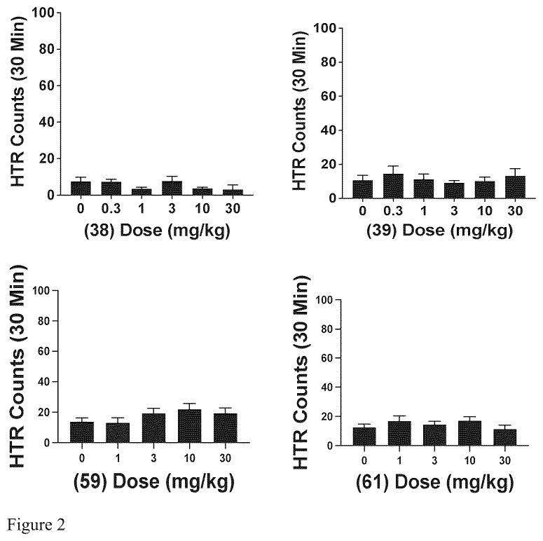

Known psychedelics such as DOI, 5-MeO-DMT, 2C—I, 25I—NBOMe, 25D-NBOMe, and LSD which induce HTR all had an Emax of 76% or higher in the BRET Gq pathway (Table 3). An unexpected finding was the relationship between the electronic effects (as determined by the Hammett op constant) of the mono-2-substituted compounds and the efficacy in the 5-HT_{2A} G protein Ca₂₊ flux assay (as well as 5-HT_{2C}). Specifically, compounds with an electron withdrawing group (for example as determined using op Hammett substitution constants with a value of >0.3) in the 2-position such as 25N—NBOCF₂H (11), 25N—NBCF₃ (14), and 25N—NBNO₂ (15) had Emax equal to or below 77% in Ca²⁺ flux assay and failed to induce a HTR in mice. This observed structure activity relationship trend around the lack of HTR with strongly electron withdrawing groups was unexpected and in contrast to compounds with electron donating groups or weakly withdrawing groups through resonance (for example as determined using Gp Hammett substitution constants with a value of <0.3) like 25N—NBOMe (4) which causes a potent HTR response and had an Emax of 94% in the Ca²⁺ flux assay. Large and sterically bulky substitutions in the 2-position (such as phenyl, thiophenyl, or O-phenyl) like 25N—NBPh (17), (59) and (61) were also G protein partial agonists (even though they are electron donating), showing an even lower Emax (in 5-HT_{2A} mediated Ca²⁺ flux and or BRET assay), and lacked a HTR in mice. Compounds containing a 3-position substitutient like (53), as well as 2,3-disubstituted compounds including 25N—NB-1-Nap (16), and 25N—NB-2-OH-3-Me (18), displayed weak Gq efficacy (Table 2 and 3) and acted as β-arrestin functionally selective compounds (showing a stronger arrestin response relative to Gq), failed to induce the HTR in mice. Similarly, the arrestin biased compounds (38), (39), which contain a Napthyl substitution failed to induce a HTR in mice greater than baseline levels in vehicle-treated mice (FIG. 2)

TABLE 8
Effect of test compounds on the head-twitch response (HTR) in mice.

| | | | Max HTR response |
|---|---|---|---|
| | | | during a 30-min |
| | | | assessment (maximum |
| | HTR ED₅₀ | | counts ÷ baseline |
| Compound | mg/kg (95% CI)¹ | μmol/kg (95% CI) | response) |
| (±)-DOI | 0.29 (0.22-0.38) | 0.80 (0.60-1.07) | 9.9-fold increase |
| 2C-I | 0.83 (0.50-1.38) | 2.42 (1.46-4.02) | 13.8-fold increase |
| 25I-NBOMe | 0.078 (0.054-0.11) | 0.17 (0.12-0.24) | 16.0-fold increase |
| 25D-NBOMe | 0.23 (0.12-0.43) | 0.64 (0.34-1.22) | 15.6-fold increase |
| 25N-NB (2) | 1.25 (0.96-1.62) | 3.53 (2.71-4.60) | 9.1-fold increase |
| 25N-NBOH (3) | 0.07 (0.04-0.11) | 0.19 (0.12-0.29) | 8.8-fold increase |
| 25N-NBOMe (4) | 0.11 (0.08-0.16) | 0.29 (0.20-0.43) | 15.6-fold increase |
| 25N-NBOEt (5) | 0.57 (0.45-0.73) | 1.44 (1.13-1.83) | 13.2-fold increase |
| 25N-NBMe (6) | 1.19 (0.49-2.91) | 3.24 (1.32-7.94) | 8.0-fold increase |
| 25N-NBF (7) | 1.52 (0.98-2.35) | 4.10 (2.65-6.33) | 4.5-fold increase |
| 25N-NBCl (8) | 4.04 (1.59-10.3) | 10.4 (4.11-26.5) | 2.3-fold increase |
| 25N-NBBr (9) | 6.31 (2.90-13.7) | 14.6 (6.71-31.8) | 4.7-fold increase |
| 25N-NBI (10) | 4.94 (3.21-7.59) | 10.3 (6.70-15.9) | 3.0-fold increase |
| 25N-NBOCF₂H (11) | 1.81 (1.23-2.67) | 4.32 (2.93-6.38) | 13.5-fold increase |
| 25N-NBOCF₃ (12) | 5.90 (2.97-11.7) | 13.5 (6.81-26.8) | 3.9-fold increase |
| 25N-NBCF₃ (14) | Inactive up to 30² | N.D. | 2.6-fold increase |
| 25N-NBNO₂ (15) | Inactive up to 100² | N.D. | 1.9-fold increase |
| 25N-N1-Nap (16) | Inactive up to 30² | N.D. | 1.2-fold increase |
| 25N-NBPh (17) | Inactive up to 100² | N.D. | 2.1-fold increase |
| 25N-NB-2-HO-3-Me (18) | Inactive up to 10² | N.D. | 1.0-fold increase |
| 25N-NB-3-HO (21) | Inactive up to 30² | N.D. | 1.7-fold increase |
| (38) | Inactive up to 30² | N.D. | 1.0-fold increase |
| (39) | Inactive up to 30² | N.D. | 1.4-fold increase |
| (53) | Inactive up to 30² | N.D. | 1.1-fold increase |
| (59) | Inactive up to 30² | N.D. | 1.6-fold increase |
| (61) | Inactive up to 30² | N.D. | 1.4-fold increase |
| (64) | Inactive up to 30² | N.D. | 2.1-fold increase |

N.D., not determined.
¹Compounds were administered SC, except for DOI and 25D-NBOMe, which were administered IP.
²Inactive when tested up to the specified dose (in mg/kg), based on the absence of significant post hoc pairwise differences between any drug group and vehicle (control)

It is proposed that 5-HT_{2A} G protein pathway partial agonists (defined as an Emax less than that of known psychedelics when compared via Emax rank order and under our assay conditions a cut off of generally 80% in Ca²⁺ flux and/or Gq BRET) do not induce a strong HTR (above baseline from vehicle control) and show attenuated, mild, or absent psychedelic activity (that is they will not induce as intense acute hallucinogenic or psychedelic experiences in humans as a compound like LSD). However, these compounds still retain sufficient signaling efficacy through G protein and/or arrestin pathways to produce desirable therapeutic effects. The 5-HT_{2A} agonists psilocybin and LSD may be useful to treat various psychiatric disorders. In addition to producing hallucinogenic and psychedelic effects, 5-HT_{2A} agonists produce other effects that may also contribute to their therapeutic efficacy, for example anti-inflammatory actions, promotion of neuroplasticity and synaptogenesis, and enhancement of mitochondrial neurogenesis. 25N—NB-2-OH-3-Me (18) and other weak-modest efficacy G protein partial agonists like 25N—NB-3-HO (21), 25N—NBOCF; (12), 25N—NBCF₃ (14), 25N—NBNO₂ (15), and (61) do not induce the HTR but still induce a relevant response (under current assay conditions a 5-HT_{2A} Emax greater than 40% but less than or equal to 80% in Ca²⁺ flux or BRET). As such, in humans, they are reasonably anticipated to not produce psychedelic effects of comparable intensity or magnitude to more efficacious agonists. However, such agents would mimic some of the therapeutic effects of hallucinogens. Such an attribute is highly desirable. Those ligands are likely to act as mixed agonist-antagonists, similar to the opioid partial agonist buprenorphine. Buprenorphine is a partial mu opioid receptor (MOR) agonist that has a superior safety profile and greater tolerability compared to full MOR agonists such as morphine and fentanyl. This profile has facilitated the successful use of buprenorphine for certain indications, including pain relief and opioid maintenance therapy.

The arrestin biased compound 25N—N1-Nap (16) antagonized PCP-induced locomotor hyperactivity in mice. Interactions with PCP-induced locomotor hyperactivity can be used to assess the antipsychotic potential of experimental medications. Existing 5-HT_{2A} antagonists, such as M100907 (a selective 5-HT_{2A} antagonist) and clozapine and olanzapine (atypical antipsychotics that act as mixed 5-HT_{2A}/D₂ antagonists), are effective at blocking the hyperlocomotion induced by dissociative drugs such as PCP and MK-801 (dizocilpine), which act as NMDA receptor antagonists. 25N—N1-Nap (16) antagonized the hyperactivity induced by PCP in mice when tested at a dose (3 mg/kg SC) that had no effect on baseline activity. M100,907, a selective 5-HT_{2A} inverse agonist, was used as a positive control, and similarly blocked the locomotor hyperactivity induced by PCP. There is the potential to use 5-HT_{2A} antagonists as medications for psychiatric disorders such as psychosis (for example, in schizophrenia, Parkinson's disease, as well as other CNS disorders and diseases) and depression. Atypical antipsychotics act in part by 5-HT_{2A} antagonism or inverse agonists, which is widely believed to contirute to their claimed superior tolerability. Pimavanserin is a 5-HT_{2A} receptor inverse agonist that is has been approved by the FDA to treat psychosis in subjects suffering from Parkinson's disease. Conventional 5-HT_{2A} antagonists and inverse agonists have not been observed to exhibit a signaling bias and we have observed the same. Although 5-HT_{2A} antagonists and inverse agonists have therapeutic efficacy in disorders such as psychosis and depression, 5-HT_{2A} blockade and inverse agonism can disrupt various neural processes required for normal brain function, including maintenance of prefrontal memory fields, generation of thalamocortical oscillatory activity, and regulation of the sleep-wake cycle. Given that 25N—N-1-Nap (16) can block the behavioral effects of DOI and PCP, but still activates the arrestin pathway, it may mimic the therapeutic effects of conventional 5-HT_{2A} antagonists and inverse agonists without disrupting normal CNS functions. Similar effects may occur with other arrestin biased 5-HT_{2A} ligands, as well as with G protein partial agonists such as 25N—NB-2-OH-3-Me (18). Notably, 25N—NB-2-OH-3-Me (18), can block the HTR in mice in a manner comparable to 25N—N1-Nap (16) and 25N—NBPh (17) and may not induce full-on hallucinogenic effects in humans (as evidenced by its inability to induce the HTR in mice) comparable to known psychedelics like LSD and DOI. Compounds such as as 25N—NB-2-OH-3-Me (18) and 25N—N-1-Nap (16) may thus have unique clinical potential compared to existing 5-HT_{2A} agonists, antagonists and inverse agonists—by exhibiting so called “mixed” agonist/antagonist properties.

### 5-HT_{2A} Arrestin-Biased Ligands Induce Internalization and Produce Tolerance

Arrestin biased compounds 25N—N-1-Nap (16) and 25N—NBPh (17) induced Arrestin-dependent internalization of 5-HT_{2A} receptors consistent with their Arrestin-biased functional activity in a NanoLuc internalization assay. A strong internalization was observed at 60 min with 5-HT, DOI and 25N—NBOMe (4) treatment. 25N—N-Nap (16) and 25N—NBPh (17), showed potent and strong internalization, consistent with their Arrestin recruitment assay potencies. The antagonist/inverse agonist Pimavanserin showed no internalization, suggesting this assay is consistent with their functional profile at Arrestin.

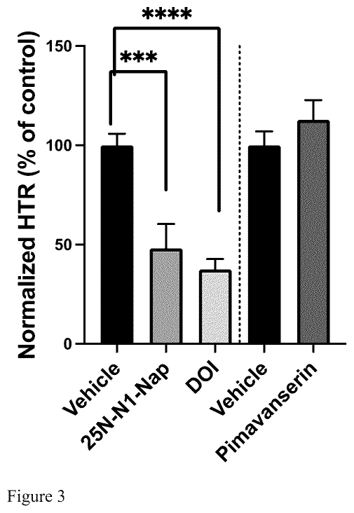

Repeat treatment with 5-HT_{2A} ligands produced effects in mice that parallel the in vitro internalization data. After repeated daily administration of 25N—N1-Nap (16) at 20 mg/kg/day (SC) or DOI at 10 mg/kg/day (SC) for 5 days, the ability of DOI (1 mg/kg IP) to induce the HTR in male C57BL/6J mice was reduced significantly (F(2.16)=16.68, p=0.0001). Conversely, the HTR induced by a challenge dose of DOI (1 mg/kg, IP) was not altered (t(10)=1.07, p=0.311) in mice treated daily (for 5 days) with a HTR-blocking dose of pimavanserin (1 mg/kg/day SC) (FIG. 3). These findings show that 5-HT_{2A} arrestin biased compounds act differently from conventional antagonists and inverse agonists as well as full unbiased agonists. This will allow unique pharmacological actions.

### Receptor Binding Experiments

Method 1: Competitive binding experiments were performed by the National Institute of Mental Health Psychoactive Drug Screening Program (NIMH PDSP) using described methods or 5-HT_{2A} experiments performed using [3H]-ketanserin displacement in human 5-HT_{2A} expressing membranes. Target compounds were dissolved in DMSO and an initial screen performed to assess displacement of the radioligand from target receptors at a concentration of 10,000 nM. The compounds that caused >50% displacement of specific radioligand binding to a given receptor then underwent secondary screenings at a range of concentrations to determine Kᵢ values. For the Kᵢ experiments, compounds were always tested in triplicate on separate plates. Each plate also contained a known ligand for the receptor as a positive control. For all 5-HT subtypes, n=3 was performed except where noted and a mean Kᵢ±SEM was calculated using these replicate experiments.

Method 2: 5HT_{2A} membrane fractions were prepared from ValiScreen Serotonin 5HT-2A (human) cell line (product No: ES-313-C) grown in DMEM/F12 media augmented with 10% FBS, 4 mM GlutaMAX, 0.4 mg/mL Geneticin, 1% Penicillin-Streptomycin. The cells were grown in a 150 mm culture dishes and were harvested between 70-90% confluency in between passages 5-15. The cells were detached with a lysis buffer (1 mM HEPES, 2 mM EDTA, pH 7.4 at room temperature) and homogenized with a hand-held homogenizer. The lysate was centrifuged for 30 minutes at 30,000× G at 4° C. The resultant pellet was resuspended in a storage buffer (20 mM HEPES, 10 mM MgCl₂, 0.1 mM EDTA, pH 7.4 at room temperature) and frozen at −80° C. The aliquots were resuspended in 10 mM HEPES at time of use. Purchased membranes, Membrane Target Systems: Serotonin 5HT-2A (human) membrane preparation, in CHO-KI cells ES-313-M400UA (Perkin Elmer), were used in lue of prepared membranes in earlier experiments for 25N—NBOMe (4) and 25N—N1-Nap (16). The purchased membranes and prepared membranes displayed comparable binding properties with reference compounds and are considered equivalent.

Suspensions of 10 mM HEPES buffer (pH 7.4 at room temperature) containing 10 μg/mL protein, 1 nM (+)-[3H]-ketanserin (Perkin Elmer NET1233, and various concentrations of unlabeled competitor or 10 μM ketanserin for nonspecific binding in a total volume of 500 μL, were incubated in the dark on a mechanical rocker at room temperature for 2 hr at 37° C. Each plate also contained multiple concentrations of ketanserin as a control. The reaction was terminated by vacuum filtration using a Unifilter-96 Cell Harvester (Perkin Elmer) over presoaked UniFilter-96 GF/C P Microplates (Perkin Elmer). Filters were washed with room temperature 10 mM HEPES buffer (pH 7.4 at room temperature) (3×1 mL). Filter plates were dried overnight and the tritium trapped on the filter measured via liquid scintillation counting with MicroScint-O (Perkin Elmer), using a MicroBeta2 Plate Reader with 6-detectors scintillation counter (Perkin Elmer) at 55% efficiency. IC₅₀ values were determined in Graphpad Prism 9.3.1 using non-linear regression (single site fit) with log-concentration plotted against percent specific binding. Percent specific binding for [3H]-ketanserin in a control experiment was ˜92%. Ki values were calculated using the equation of Cheng and Prusoff. The K_{d} for ketanserin (7.79 nM), was determined via a homologous binding experiment. Protein concentration was determined via the Bradford method using Coomassie protein assay reagent (Sigma, USA) with Bovine Serum albumin (Sigma, USA) as standard. Experiments were performed in duplicate and repeated a minimum of two times.

Method 2: 5HT_{2A} membrane fractions were prepared from ValiScreen Serotonin 5HT-2A (human) cell line (product No: ES-313-C) grown in DMEM/F12 media augmented with 10% FBS, 4 mM GlutaMAX, 0.4 mg/mL Geneticin, 1% Penicillin-Streptomycin. The cells were grown in a 150 mm culture dishes and were harvested between 70-90% confluency in between passages 5-15. The cells were detached with a lysis buffer (1 mM HEPES, 2 mM EDTA, pH 7.4 at room temperature) and homogenized with a hand-held homogenizer. The lysate was centrifuged for 30 minutes at 30,000 G at 4° C. The resultant pellet was resuspended in a storage buffer (20 mM HEPES, 10 mM MgCl2, 0.1 mM EDTA, pH 7.4 at room temperature) and frozen at −80° C. The aliquots were resuspended in 10 mM HEPES at time of use. Purchased membranes, Membrane Target Systems: Serotonin 5HT-2A (human) membrane preparation, in CHO-KI cells ES-313-M400UA (Perkin Elmer), were used in lue of prepared membranes in earlier experiments for 25N—NBOMe (4) and 25N—N-Nap (16). The purchased membranes and prepared membranes displayed comparable binding properties with reference compounds and are considered equivalent.

Suspensions of 10 mM HEPES buffer (pH 7.4 at room temperature) containing 10 μg/mL protein, 1 nM (+)-[3H]-ketanserin (Perkin Elmer NET1233, and various concentrations of unlabeled competitor or 10 μM Ketanserin for nonspecific binding in a total volume of 500 μL, were incubated in the dark on a mechanical rocker at room temperature for 2 hr at 37° C. Each plate also contained multiple concentrations of ketanserin as a control. The reaction was terminated by vacuum filtration using a Unifilter-96 Cell Harvester (Perkin Elmer) over presoaked UniFilter-96 GF/C P Microplates (Perkin Elmer). Filters were washed with room temperature 10 mM HEPES buffer (pH 7.4 at room temperature) (3×1 mL). Filter plates were dried overnight and the tritium trapped on the filter measured via liquid scintillation counting with MicroScint-O (Perkin Elmer), using a MicroBeta2 Plate Reader with 6-detectors scintillation counter (Perkin Elmer) at 55% efficiency. IC₅₀ values were determined in Graphpad Prism 9.3.1 using non-linear regression (single site fit) with log-concentration plotted against percent specific binding. Percent specific binding for [3H]-Ketanserin in a control experiment was ˜92%. Kᵢ values were calculated using the equation of Cheng and Prusoff. The Kd for Ketanserin (7.785 nM), was determined via a homologous binding assay and is consistent with literature values. Protein concentration was determined via the Bradford method using Coomassie protein assay reagent (Sigma, USA) with Bovine Serum albumin (Sigma, USA) as standard. Experiments were performed in duplicate and repeated a minimum of two times.

### BRET Assays for Gq Dissociation and β-Arrestin Recruitment

To measure 5-HT receptor-mediated β-Arrestin recruitment by BRET1, HEK293T cells were co-transfected in a 1:15 ratio with human or mouse 5-HT receptors containing a C-terminal fused renilla luciferase (RLuc8), and a Venus-tagged N-terminal β-arrestin using 3:1 ratio of TransiT-2020 (Mirus) in DMEM supplemented with 10% dialyzed FBS (Omega Scientific) and. To measure 5-HT receptor-mediated Gq activation via Gq/γl dissociation by BRET², HEK293T cells were co-transfected in a 1:1:1:1 ratio with RLuc8-fused human Gαq (Gαq-RLuc8), a GFP²-fused to the C-terminus of human Gγl(Gγl-GFP²), human Gβ1, and 5-HT receptor using TransiT-2020 in DMEM supplemented with 10% dialyzed FBS, as described previously (Nat Str and Mol Biology 2018 September: 25(9): 787-796. Epub 2018 Aug. 20)). After at least 18-24 hours, transfected cells were plated in poly-lysine coated 96-well white clear bottom cell culture plates in DMEM containing 1% dialyzed FBS at a density of 25-40,000 cells in 200 μL per well and incubated overnight. After approximately 20-24 hours, media was decanted and cells were washed with 60 μL of drug buffer (1×HBSS, 20 mM HEPES, pH 7.4), followed by 60 μL of drug buffer and pre-incubated in a humidified atmosphere at 37° C. before receiving drug stimulation. Drug stimulation was induced by adding 30 μL of a solution of drug (3X) diluted in McCorvy buffer (1×HBSS, 20 mM HEPES, pH 7.4, supplemented with 0.3% BSA fatty acid free, 0.03% ascorbic acid) and plates were incubated at the indicated time and temperature. Fifteen minutes before reading, 10 μL of the RLuc substrate, either coelenterazine h for β-Arrestin recruitment BRET¹ or coelenterazine 400a for Gq dissociation BRET² (Prolume/Nanolight, 5 μM final concentration) was addedper well. Plates were read for luminescence at 485 nm and fluorescent eYFP emission at 530 nm for BRET¹ and at 400 nm and fluorescent GFP2 emission at 510 nm for BRET2 at 1 second per well using a Mithras LB940 (Berthold). The BRET ratios of fluorescence/luminescence were calculated per well and were plotted as a function of drug concentration using Graphpad Prism 8 (Graphpad Software Inc., San Diego, CA). Data were normalized to % 5-HT stimulation and analyzed using the nonlinear regression model “log(agonist) vs. response” to yield Emax and EC50 parameter estimates.

### Calcium Flux Assays

Calcium flux was measured using stable-expressing 5-HT₂ Flp-In 293 T-Rex Tetracycline inducible system described previously (Investigation of the Structure-Activity Relationships of Psilocy bin Analogues, ACS Pharmacol. Transl. Sci. 2020, Publication Date: Dec. 14, 2020, https://doi.org/10.1021/acsptsci.0c00176). Cell lines were maintained in DMEM containing 10% FBS, 10 μg/mL Blasticidin (Invivogen), and 100 μg/mL Hygromycin B (GoldBio). At least 20-24 hours before the assay, receptor expression was induced with tetracycline (2 μL/mL) and cells were seeded into 384-well poly-L-lysine-coated black plates at a density of 7,500 cells/well in DMEM containing 1% dialyzed FBS. On the day of the assay, the cells were incubated for 1 h at 37° C. with Fluo-4 Direct dye (Invitrogen, 20 μl/well) reconstituted in drug buffer (20 mM HEPES-buffered HBSS, pH 7.4) containing 2.5 mM probenecid. Drug dilutions were prepared at 5X final concentration in McCorvy buffer (20 mM HEPES-buffered HBSS, 0).1% BSA, 0.01% ascorbic acid pH 7.4). After dye load, cells were allowed to equilibrate to room temperature for 15 minutes, and then placed in a FLIPR^{TETRA} fluorescence imaging plate reader (Molecular Devices). Fluorescence for the FLIPR^{TETRA} were programmed to read baseline fluorescence for 10 s (1 read/s), and afterward 5 μl of drug per well was added and read for a total of 5-10 min (1 read/s). Fluorescence in each well was normalized to the average of the first 10 reads for baseline fluorescence, and then either maximum-fold peak increase over basal or area under the curve (AUC) was calculated. Either peak or AUC was plotted as a function of drug concentration, and data were normalized to percent 5-HT stimulation. Data was plotted and non-linear regression was performed using “log(agonist) vs. response” in Graphpad Prism 8 to yield Emax and EC₅₀ parameter estimates.

### Animal Behavioral Experiments

Male C57BL/6J mice (6-8 weeks old) from Jackson Labs (Bar Harbor, ME, USA) were used for the behavioral experiments. The mice were housed on a reversed light-dark cycle (lights on at 1900 h, off at 0700 h) in an AAAC-approved vivarium at the University of California San Diego. Mice were housed up to four per cage in a climate-controlled room and with food and water provided ad libitum except during behavioral testing. Testing was performed between 1000 and 1800 h (during the dark phase of the light-dark cycle). The studies were conducted in accordance with National Institutes Health (NIH) guidelines and were approved by the University of California San Diego Institutional Animal Care and Use Committee.

The drug solutions used for the behavioral experiments were prepared as follows: YM-254,890 (FUJIFILM Wako Chemicals USA, Richmond, VA, USA) was dissolved in 100% dimethyl sulfoxide (DMSO): edelfosine (Tocris Bioscience, Minneapolis, MN, USA), (±)-2,5-dimethoxy-4-iodoamphetamine hydrochloride (DOI: Cayman Chemical, Ann Arbor, MI, USA), R—(−)-2,5-dimethoxy-4-iodoamphetamine hydrochloride (R—(−)-DOI: donated by the National Institute on Drug Abuse, Rockville, MD, USA), phencyclidine hydrochloride (PCP: Sigma-Aldrich), 25N—NBOMe hydrochloride, and 25N—NB hydrochloride were dissolved in isotonic saline: 25N-NBOEt hydrochloride, 25N—NB-2-OH-3-Me hydrochloride, and 25N—NBPh hydrochloride were dissolved in distilled water containing 5% Tween-80 (v/v): 25N—NBNO₂ hydrochloride was dissolved in distilled water containing 20% hydroxypropyl-β-cyclodextran (w/v): 25N—NBBr hydrochloride was dissolved in distilled water containing 5% Tween-80 (v/v) and 20% hydroxypropyl-β-cyclodextran (w/v): for the remaining compounds, the hydrochloride salts were dissolved in distilled water containing 1% Tween-80 (v/v). Edelfosine, DOI, R—(−)-DOI, and PCP were injected IP (5 mL/kg): 25N—NB and its derivatives were injected SC (5 mL/kg or 10 mL/kg): YM-254,890 was injected into the lateral ventricle (2 μL) over 1 min.

### Assessment of the Head-Twitch Response

The head-twitch response (HTR) was assessed using a head-mounted neodymium magnet and a magnetometer detection coil, as described previously (Halberstadt and Geyer, 2013). The mice were allowed to recover from the magnet implantation surgeries for at least 1 week prior to behavioral testing. HTR experiments were conducted in a well-lit room, and the mice were allowed to habituate to the room for at least 1 h prior to testing. Head twitches was assessed in a 12-cm diameter glass cylinder surrounded by a magnetometer coil. Coil voltage was low-pass filtered (2 kHz), amplified, and digitized (20-kHz sampling rate) using a Powerlab/8SP with LabChart v 7.3.2 (ADInstruments, Colorado Springs, CO, USA). The data were filtered offline (40-200-Hz band-pass) and head twitches were identified by their waveform characteristics using established criteria (Halberstadt and Geyer, 2014). HTR counts were analyzed using one-way ANOVAs. Dunnett's test was used for post hoc comparisons. Significance was demonstrated by surpassing an a level of 0.05. Median effective doses (ED50 values) and 95% confidence intervals for dose-response experiments were calculated by nonlinear regression (Prism 7.00, GraphPad Software, San Diego, CA, USA).

### Assessment of PCP-Induced Locomotor Activity

The mouse behavioral pattern monitor (BPM) was used to assess locomotor activity. Each mouse BPM chamber (San Diego Instruments, San Diego, CA, USA) is a transparent Plexiglas box with an opaque 30×60 cm floor, enclosed in a ventilated isolation box. The position of the mouse in x.y coordinates is recorded by a grid of 12×24 infrared photobeams located 1 cm above the floor. A second row of 16 photobeams (parallel to the long axis of the chamber, located 2.5 cm above the floor) is used to detect rearing behavior. Holepoking behavior is detected by 11 1.4-cm holes that are situated in the walls (3 holes in each long wall, 2 holes in each short wall) and the floor (3 holes); each hole is equipped with an infrared photobeam. The status of each photobeam is sampled every 55 ms and recorded for offline analysis. Locomotor activity was quantified as distance traveled, which was analyzed in 20-min blocks using a three-way ANOVA, with pretreatment and treatment as between subject variables and time as a within-subject variable. Tukey's studentized range method was used for post hoc comparisons. Significance was demonstrated by surpassing an a level of 0.05.

It will be appreciated by persons skilled in the art that the invention described herein is not limited to what has been particularly shown and described. Rather, the scope of the invention is defined by the claims which follow. It should further be understood that the above description is only representative of illustrative examples of embodiments. The description has not attempted to exhaustively enumerate all possible variations. The alternate embodiments may not have been presented for a specific substituent of the compound, or a step of the method, and may result from a different combination of described substituent or step, or that other undescribed alternate embodiments may be available for a compound or method, is not to be considered a disclaimer of those alternate embodiments. It will be appreciated that many of those un-described embodiments are within the literal scope of the following claims, and others are equivalent.

1. A compound of formula (I) or a pharmaceutically acceptable salt thereof, 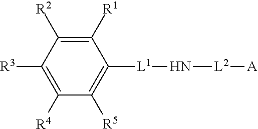
Wherein:
R¹, R², R³, R⁴ and R⁵ are independently selected from the group consisting of hydrogen, deuterium, OC₁₋₆alkyl, SC₁₋₆alkyl, CN, OH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NRmSO₂C₁₋₆alkyl, C₁₋₆alkylSO₂ (sulfone), S(O)OH, C₁₋₆alkylS(O) (sulfoxide), nitroso, C₁₋₆alkylOSO₂, 3-10 membered heterocycloalkyl, 6-10 membered aryl, 5-10 membered heteroaryl, C₁₋₆alkylene-N(R^{m})₂, C₁₋₆alkylene-N(R^{m})(COR^{m});
provided that at least one of R¹ and R² contains an oxygen bonded to the phenyl ring;
A is 4, 5, 6 or 7 membered ring optionally substituted with one or more substituents selected from the group consisting of OC₁₋₆alkyl, SC₁₋₆alkyl, CN, OH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, C₃₋₆cycloalkyl, 3-10 membered heterocycloalkyl, 6-10 membered aryl, 5-10 membered heteroaryl, 5-12 membered bicycloalkyl, and 5-12 membered hetero-bicycloalkyl, wherein the C₃₋₆cycloalkyl, 3-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl_{6-10-membered}, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroaryl_{5-10-membered}, OC₁₋₂alkylene-bicycloalkyl_{5-12-membered}, OC₁₋₂alkylene-hetero-bicycloalkyl_{5-12-membered}, C₁₋₂alkylene-C₃₋₆cycloalkyl, C₁₋₂alkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, C₁₋₂alkylene-hetero-bicycloalkyl_{5-12-membered}, wherein each of these rings is optionally substituted with one or more substituents selected from the group consisting of OC₁₋₆alkyl, SC₁₋₆alkyl, CN, OH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, C(=NC₁₋₆alkyl)C₁₋₆alkyl, OC(O)N(R^{m})₂, SH, C(O)SRm, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NRmSO₂C₁₋₆alkyl, C₁₋₆alkylSO₂ (sulfone), S(O)OH, C₁₋₆alkylS(O) (sulfoxide), nitroso, and C₁₋₆alkylOSO₂;
alternatively, two adjacent substituents of A link up and together with A form a bicyclic or tricylic ring;
R^{m} each is independently hydrogen or C₁₋₆alkyl or halo-C₁₋₆alkyl;
L¹ is C₁₋₃alkylene, optionally R¹ and L¹ link up to form a ring; and
L² is a bond or C₁₋₃alkylene optionally substituted with C₁₋₄alkyl, C₃₋₆ cycloalkyl, haloC₁₋₄alkyl, deuterium or F,
provided that the compound is not 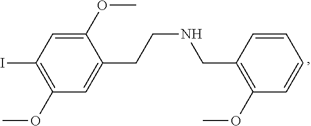 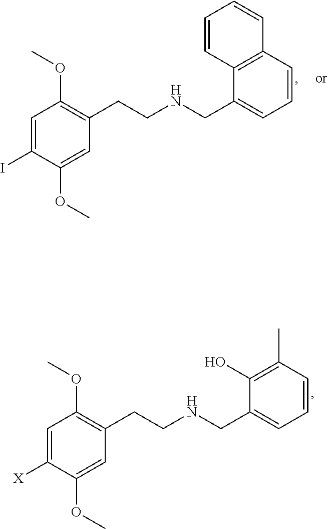
wherein X is CN, Cl, Br or I.

2. The compound of formula (I) or the pharmaceutically acceptable salt thereof of claim 1, wherein R¹ is OC₁₋₃alkyl.

3. The compound of formula (I) or the pharmaceutically acceptable salt thereof of claim 1, wherein R² is OC₁₋₃alkyl.

4. The compound of formula (I) or the pharmaceutically acceptable salt thereof of claim 1, wherein R⁴ is OC₁₋₃alkyl.

5. The compound of formula (I) or the pharmaceutically acceptable salt thereof of claim 1, wherein R⁶ is OC₁₋₃alkyl.

6. The compound of formula (I) or the pharmaceutically acceptable salt thereof of claim 1, wherein
(a) R¹ and R⁴ are each independently OC₁₋₃alkyl;
(b) R¹ and R⁵ are each independently OC₁₋₃alkyl; or
(c) R² and R⁵ are each independently OC₁₋₃alkyl.

7. The compound of formula (I) or the pharmaceutically acceptable salt thereof of claim 1, wherein R³ is selected from the group consisting of OC₁₋₆alkyl, SC₁₋₆alkyl, CN, halogen, NO₂, N(R^{m})₂, haloC₁₋₆alkyl, and C₁₋₆alkyl, and R⁴ is selected from the group consisting of hydrogen, OC₁₋₆alkyl, SC₁₋₆alkyl, CN, OH, halogen, NO₂, N(R^{m})₂, haloC₁₋₆alkyl, and C₁₋₆alkyl.

8. The compound of formula (I) or the pharmaceutically acceptable salt thereof of claim 1, wherein R¹ and R⁴ are each independently OC₁₋₃alkyl; R³ is NO₂, haloC₁₋₆alkyl, or C₁₋₆alkyl.

9. The compound of formula (I) or the pharmaceutically acceptable salt thereof of claim 1, wherein L¹ is ethylene.

10. The compound of formula (I) or the pharmaceutically acceptable salt thereof of claim 1, wherein L² is methylene.

11. The compound of formula (I) or the pharmaceutically acceptable salt thereof of claim 1, wherein two adjacent substituents of A link up and together with A form a bicyclic ring or tricyclic ring.

12. The compound of formula (I) or the pharmaceutically acceptable salt thereof of claim 1, wherein two adjacent substituents of A link up and together with A form a bicyclic ring selected from the group consisting of indanyl, 1,2,3,4-tetrahydronaphthalenyl, benzimidazolyl, benzofuranyl, benzoselenophene, benzothiofuranyl, benzothiophenyl, benzoxazolyl, benzthiazolyl, benztriazolyl, benztetrazolyl, benzisoxazolyl, benzisothiazolyl, benzimidazolinyl, chromanyl, chromenyl, cinnolinyl, indolenyl, indolinyl, indolizinyl, indolyl, 3H-indolyl, indazolyl, isobenzofuranyl, isoindazolyl, isoindolinyl, isoindolyl, isoquinolinyl, methylenedioxyphenyl, naphthyridinyl, naphthalenyl, octahydroisoquinolinyl, quinazolinyl, quinolinyl, 4H-quinolizinyl, quinoxalinyl, tetrahydroisoquinolinyl, and tetrahydroquinolinyl, wherein the bicyclic ring is optionally substituted with one or more substituents selected from the group consisting of OC₁₋₆alkyl, SC₁₋₆alkyl, CN, OH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, and dihydroxyC₁₋₁₀alkyl.

13. The compound of formula (I) or the pharmaceutically acceptable salt thereof of claim 1, wherein A is represented as 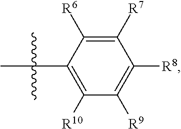
Wherein R⁶ and R⁷ are independently selected from the group consisting of H, OC₁₋₆alkyl, SC₁₋₆alkyl, CN, OH, halogen, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NRmSO₂C₁₋₆alkyl, C₁₋₆alkylSO₂ (sulfone), S(O)OH, C₁₋₆alkylS(O) (sulfoxide), nitroso, C₁₋₆alkylOSO₂, C₃₋₆cycloalkyl, 3-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl_{6-10-membered}, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroaryl_{5-10-membered}, OC₁₋₂alkylene-bicycloalkyl_{5-12-membered}, OC₁₋₂alkylene-hetero-bicycloalkyl_{5-12-membered}, C₁₋₂alkylene-C₃₋₆cycloalkyl, C₁₋₂alkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, and C₁₋₂alkylene-hetero-bicycloalkyl_{5-12-membered}, wherein each of the ring is optionally substituted; provided that at least one of R⁶ and R⁷ is not H;
R⁸, R⁹ and R¹⁰ are independently selected from the group consisting of H, OC₁₋₆alkyl, SC₁₋₆alkyl, CN, OH, halogen, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂.

14. The compound of formula (I) or the pharmaceutically acceptable salt thereof of claim 13, wherein R⁶ is selected from the group consisting of C₃₋₆cycloalkyl, 3-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl_{6-10-membered}, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroaryl_{5-10-membered}, OC₁₋₂alkylene-bicycloalkyl_{5-12-membered}, OC₁₋₂alkylene-hetero-bicycloalkyl_{5-12-membered}, C₁₋₂alkylene-C₃₋₆cycloalkyl, C₁₋₂alkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, C₁₋₂alkylene-hetero-bicycloalkyl_{5-12-membered}, wherein each of the rings is optionally substituted.

15. The compound of formula (I) or the pharmaceutically acceptable salt thereof of claim 13, wherein R⁶ is an optionally substituted ring system selected from the group consisting of adamantanyl, cyclopropyl, cyclobutyl, cyclopentyl, cyclopentenyl, cyclohexyl, cyclohexenyl, cycloheptyl, cyclooctyl, tetrahydrofuranyl, ferrocenyl, furanyl, furazanyl, imidazolinyl, imidazolyl, norbornyl, norbornenyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, phenyl, piperazinyl, pyrimidinyl, piperonyl, pyranyl, pyrazinyl, pyrazolidinyl, pyrazolinyl, pyrazolyl, pyridazinyl, pyridinyl, pyridyl, pyrimidinyl, pyrrolinyl, 2H-pyrrolyl, pyrrolyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl.

16. The compound of formula (I) or the pharmaceutically acceptable salt thereof of claim 13, wherein R⁶ is selected from the group consisting of H, OC₁₋₆alkyl, SC₁₋₆alkyl, CN, OH, halogen, N(R^{m})₂, C(O)OR^{m}), C(O)N(R^{m})₂, C(O)C₁₋₆alkyl, haloC₁₋₆alkyl, haloC₁₋₆alkyleneO, C₁₋₆alkyl, and hydroxyC₁₋₆alkyl; and
R⁷ is selected from the group consisting of H, OC₁₋₆alkyl, SC₁₋₆alkyl, CN, OH, halogen, N(R^{m})₂, C(O)C₁₋₆alkyl, haloC₁₋₆alkyl, haloC₁₋₆alkyleneO, C₁₋₆alkyl, hydroxyC₁₋₆alkyl, C₃₋₆cycloalkyl, C₃₋₆cycloalkyl, 3-10 membered heterocycloalkyl, 6-10 membered aryl, 5-10 membered heteroaryl, 5-12 membered bicycloalkyl, 5-12 membered hetero-bicycloalkyl, O—C₃₋₆cycloalkyl, O-heterocycloalkyl_{3-10-membered}, O-aryl_{6-10-membered}, O-heteroaryl_{5-10-membered}, O-bicycloalkyl_{5-12-membered}, O-hetero-bicycloalkyl_{5-12-membered}, OC₁₋₂alkylene-C₃₋₆cycloalkyl, OC₁₋₂alkylene-heterocycloalkyl_{3-10-membered}, OC₁₋₂alkylene-aryl_{6-10-membered}, OC₁₋₂alkylene-heteroaryl_{5-10-membered}, OC₁₋₂alkylene-bicycloalkyl_{5-12-membered}, OC₁₋₂alkylene-hetero-bicycloalkyl_{5-12-membered}, C₁₋₂alkylene-C₃₋₆cycloalkyl, C₁₋₂alkylene-heterocycloalkyl_{3-10-membered}, C₁₋₂alkylene-aryl_{6-10-membered}, C₁₋₂alkylene-heteroaryl_{5-10-membered}, C₁₋₂alkylene-bicycloalkyl_{5-12-membered}, C₁₋₂alkylene-hetero-bicycloalkyl_{5-12-membered}, wherein each of the ring is optionally substituted.

17. The compound of formula (I) or the pharmaceutically acceptable salt thereof of claim 13, wherein R⁸ is selected from the group consisting of OC₁₋₆alkyl, SC₁₋₆alkyl, CN, OH, halogen, N(R^{m})₂, haloC₁₋₆alkyl, haloC₁₋₆alkyleneO, C₁₋₆alkyl, C(═NC₁₋₆alkyl)C₁₋₆alkyl, OC(O)N(R^{m})₂, SH, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R¹¹)₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂.

18. The compound of formula (I) or the pharmaceutically acceptable salt thereof of claim 1, which is represented by 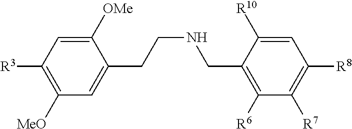
Wherein

| Compound | R⁶ | R⁷ | R⁸ | R¹⁰ | R³ |
|---|---|---|---|---|---|
| 25N-NBOH | OH | H | H | H | NO₂ |
| (3) | | | | | |
| 25N-NBOMe | OCH₃ | H | H | H | NO₂ |
| (4) | | | | | |
| 25N-NBOEt | OCH₂CH₃ | H | H | H | NO₂ |
| (5) | | | | | |
| 25N-NBMe | CH₃ | H | H | H | NO₂ |
| (6) | | | | | |
| 25N-NBF | F | H | H | H | NO₂ |
| (7) | | | | | |
| 25N-NBCl | Cl | H | H | H | NO₂ |
| (8) | | | | | |
| 25N-NBBr | Br | H | H | H | NO₂ |
| (9) | | | | | |
| 25N-NBI | I | H | H | H | NO₂ |
| (10) | | | | | |
| 25N-NBOCF₂H | OCF₂H | H | H | H | NO₂ |
| (11) | | | | | |
| 25N-NBOCF₃ | OCF₃ | H | H | H | NO₂ |
| (12) | | | | | |
| 25N-NBMDF₂ | OCF₂O | | H | H | NO₂ |
| (13) | | | | | |
| 25N-NBCF₃ | CF₃ | H | H | H | NO₂ |
| (14) | | | | | |
| 25N-NBNO₂ | NO₂ | H | H | H | NO₂ |
| (15) | | | | | |
| 25N-N-1-Nap | (CH)₄ | | H | H | NO₂ |
| (16) | | | | | |
| 25N-NBPh | Ph | H | H | H | NO₂ |
| (17) | | | | | |
| 25N-NB-2-OH-3-Me | OH | CH₃ | H | H | NO₂ |
| (18) | | | | | |
| 25N-NB-2-MeO-3-F | OCH₃ | F | H | H | NO₂ |
| (19) | | | | | |
| 25N-NB-2,5-DiMeO | OCH₃ | H | H | OCH₃ | NO₂ |
| (20) | | | | | |
| 25N-NB-3-OH | H | OH | H | H | NO₂ |
| (21) | | | | | |
| 25N-NB-3-Me | H | CH₃ | H | H | NO₂ |
| (22) | | | | | |
| 25N-NB-4-Me | H | H | CH₃ | H | NO₂ |
| (23) | | | | | |
| 25N-NB-3-F | H | F | H | H | NO₂ |
| (24) | | | | | |
| 25N-NB-4-F | H | H | F | H | NO₂ |
| (25) | | | | | |
| 25D-NBOMe | OCH₃ | H | H | H | CH₃ |
| (26) | | | | | |
| 25D-N1-Nap | (CH)₄ | | H | H | CH₃ |
| (27) | | | | | |
| 25D-NBPh | Ph | H | H | H | CH₃ |
| (28) | | | | | |

19. A pharmaceutical composition comprising the compound of formula (I) or the pharmaceutically acceptable salt thereof of claim 1 and a pharmaceutically acceptable carrier.

20. A method of treating a disease or condition, comprising administering to a subject in need thereof the compound of formula (I) or the pharmaceutically acceptable salt thereof of claim 1.

21. The method of claim 20, wherein the disease or condition is a psychiatric or neurological disease or condition or sign or symptom selected from the group consisting of attention deficient disorder, attention deficit hyperactivity disorder (ADHD), adult attention-deficit/hyperactivity disorder, learning disorders, neurocognitive disorders, Tic disorders, autism spectrum disorder, Tourette's disorder, schizophrenia, negative symptoms of schizophrenia, cognitive symptoms of schizophrenia, substance/medication-induced psychotic disorder, psychotic disorder due to another medical condition, brief psychotic disorder, schizophreniform disorder, schizoaffective disorder, disruptive mood dysregulation disorder, depression, post-partum depression, persistent depressive disorder, major depressive episode, major depressive disorder, treatment-resistant depression, post-traumatic stress disorder, reactive attachment disorder, disinhibited social engagement disorder, personality disorders, psychopathy, cyclothymic disorder, manic episode, hypomanic episode, bipolar disorder, delusional disorder, obsessive compulsive disorder, hoarding disorder, premenstrual dysphoric disorder, somatic symptom and related disorders, intellectual disabilities, communication disorders, motor disorders, catalepsy, catatonia, agitation, hypertension, sleep disorders, sexual dysfunctions anxiety disorders, adjustment disorders, body dysmorphic disorder, Trichotillomania, excoriation disorder, substance/medication-induced obsessive-compulsive and related disorder, dementias, neurodegenerative diseases, seasonal affective disorder, pseudobulbar affect, cluster headache, headaches, migraines, Tension-type headaches, tinnitus, hallucinations, delusions, epilepsies, cyclic vomiting syndrome, cannabinoid hyperemesis, nausea, restless leg syndrome, weight loss or binge eating, anorexia nervosa, bulimia nervosa, alcoholism, nicotine dependence, substance use disorders, non-substance related disorders, oppositional defiant disorder, intermittent explosive disorder, conduct disorder, pyromania, kleptomania, paraphilic disorders, medication induced movement disorders, and adverse effects of other medications.

22. The method of claim 20, wherein the disease or condition is selected from the group consisting of autoimmune diseases, acute pain, chronic pain, neuropathic pain, cancer, cough, infections, tinnitus, hearing loss, loss of taste, loss of smell, endocrine diseases and disorders, diabetes, gastrointestinal tract related diseases, urinary tract diseases, blood diseases, cardiovascular disease, inflammatory diseases, arthritis, paralysis, or spinal cord injury.

23. (canceled)

24. (canceled)

25. (canceled)

26. The method of claim 20, wherein the subject has taken a hallucinogen.

27. (canceled)

28. (canceled)

29. (canceled)

30. (canceled)

31. (canceled)
